SEQUENCE LISTING (108 sequences)

SEQ ID NO: 1 - protein, 87 aa, Saccharomyces cerevisiae
MVSQLFEEKAKAVNELPTKPSTDELLELYALYKQATVGDNDKEKPGIFNMKDRYKWEAWENLKGKSQEDAEKEYIALVDQLIAKYSS

SEQ ID NO: 2 - protein, 92 aa, Brassica napus
MGLKEDFEEHAEKVKKLTASPSNEDLLILYGLYKQATVGPVTTSRPGMFSMKERAKWDAWKAVEGKSTDEAMSDYITKVKQLLEAEASSASA

SEQ ID NO: 3 - protein, 89 aa, Gossypium hirsutum
MGLKEEFEEHAEKVKTLPAAPSNDDMLILYGLYKQATVGPVNTSRPGMFNMREKYKWDAWKAVEGKSKEEAMGDYITKVKQLFEAAGSS

SEQ ID NO: 4 - protein, 90 aa, Ricinus communis
MGLKEDFEEHAEKAKTLPENTTNENKLILYGLYKQATVGPVNTSRPGMFNMRDRAKWDAWKAVEGKSTEEAMSDYITKVKQLLGEAAASA

SEQ ID NO: 5 - protein, 92 aa, Arabidopsis thaliana
MGLKEEFEEHAEKVNTLTELPSNEDLLILYGLYKQAKFGPVDTSRPGMFSMKERAKWDAWKAVEGKSSEEAMNDYITKVKQLLEVAASKAST

SEQ ID NO: 6 - protein, 90 aa, Hyacinthus orientalis
GGVRRAHGSIATWAGTAASDEKKLMLYGLFKQATVGPINIDRPAITSLKDRAKWDAWKAVEAKTKDEAMSEYIAIVKKLLEGVMDANRTA

SEQ ID NO: 7 - protein, 676 aa, Arabidopsis thaliana
MADWYQLAQSIIFGLIFAYLLAKLISILLAFKDENLSLTRNHTTQSEYENLRKVETLTGISGETDSLIAEQGSLRGDEDESDDDDWEGVESTELDEAFSAATAFVAAAASDRLSQKVSNELQLQLYGLYKIATEGPCTAPQPSALKMTARAKWQAWQKLGAMPPEEAMEKYIDLVTQLYPAWVEGGSKRRNRSGEAAGPMGPVFSSLVYEEESDNELKIDAIHAFAREGEVENLLKCIENGIPVNARDSEGRTPLHWAIDRGHLNVAEALVDKNADVNAKDNEGQTSLHYAVVCEREALAEFLVKQKADTTIKDEDGNSPLDLCESEWSWMREKKDSNMADWYQLAQSIIFGLIFAYLLAKLISILLAFKDENLSLTRNHTTQSEYENLRKVETLTGISGETDSLIAEQGSLRGDEDESDDDDWEGVESTELDEAFSAATAFVAAAASDRLSQKVSNELQLQLYGLYKIATEGPCTAPQPSALKMTARAKWQAWQKLGAMPPEEAMEKYIDLVTQLYPAWVEGGSKRRNRSGEAAGPMGPVFSSLVYEEESDNELKIDAIHAFAREGEVENLLKCIENGIPVNARDSEGRTPLHWAIDRGHLNVAEALVDKNADVNAKDNEGQTSLHYAVVCEREALAEFLVKQKADTTIKDEDGNSPLDLCESEWSWMREKKDSN

SEQ ID NO: 8 - protein, 184 aa, Oryza sativa
MGLQEDFEQYAEKGRPCRRALATRTSLSSMDSTSRPPLEMSILLVLAYSPRGTGRNGMHGKLLKANRRRKQLSDYITKVKQLLEEACCLQLLMGLQEDFEQYAEKGRPCRRALATRTSLSSMDSTSRPPLEMSILLVLAYSPRGTGRNGMHGKLLKANRRRKQLSDYITKVKQLLEEACCLQLL

SEQ ID NO: 9 - protein, 145 aa, Aspergillus clavatus
MASLTDFFTAFDAAATKQKFPASLQSSAAAIDKAALQAAVEAVLAGGDDATAGAQDAVLKAGFEFATELVKMLEKEPGPEEKLALYKYFKQARGEQPAQPSFYQMEAKFKYNAWKEVSHISAQKAQALYIKEVNELINKYGTRAE

SEQ ID NO: 10 - protein, 562 aa, Oryza sativa
MELFYELLLTAAASLLVAFLLARLLASAATASDPRRRAPDHAAVIAEEEAVVVEEERIIEVDEVEVKSARARECVVSEGWVEVGRASSAEGKLECLPEEEEAPAKAARELVLDAVLEEREEEGQVGEERCDLAAAVAEVVGVKPHELGVEAAPGEVSDVTLEEGKVQDVGVEQHDLVAEAAPREALDTGLEKQGVPIIEAVEIKRQDDLGAEVAPSDVPEVEFEQQGVRIIEAIDVNQHHRVALAAPAEVVDAGLEERVQAIEAGSSGLTSETVPEEVLDELSEKQEEQVIEEKEHQLAAATAPVAIPGVALAETEELKEEQSSEKAVNVHEEVQSKDEAKCKLHLVDQQEGSASKVELVGRNTDNVEISHGSSSGDKMIAELTEEELTLQGVPADETQTDMEFGEWEGIERTEIEKRFGVAAAFASSDAGMAALSKLDSDVQLQLQGLLKVAIDGPCYDSTQPLTLRPSSRAKWAAWQKLGNMYPETAMERYMNLLSEAIPGWMGDNISGTKEHEAGDDAVGSVLTMTSNTINQHDSQGNEDNTGMYEGHLTSSPNPEKEF

SEQ ID NO: 11 - protein, 569 aa, Oryza sativa
MELFYELLLTAAASLLVAFLLARLLASAATASDPRRRAPDHAAVIAEEEAVVVEEERIIEVDEVEVKSARARECVVSEGWVEVGRASSAEGKLECLPEEEEAPAKAARELVLDAVLEEREEEGQVGEERCDLAAAVAEVVGVKPHELGVEAAPGEVSDVTLEEGKVQDVGVEQHDLVAEAAPREALDTGLEKQGVPIIEAVEIKRQDDLGAEVAPSDVPEVEFEQQGVRIIEAIDVNQHHRVALAAPAEVVDAGLEERVQAIEAGSSGLTSETVPEEVLDELSEKQEEQVIEEKEHQLAAATAPVAIPGVALAETEELKEEQSSEKAVNVHEEVQSKDEAKCKLHLVDQQEGSASKVELVGRNTDNVEISHGSSSGDKMIAELTEEELTLQGVPADETQTDMEFGEWEGIERTEIEKRFGVAAAFASSDAGMAALSKLDSDVQLQLQGLLKVAIDGPCYDSTQPLTLRPSSRAKWAAWQKLGNMYPETAMERYMNLLSEAIPGWMGDNISGTKEHEAGDDAVGSVLTMTSNTINQHDSQGNEDNTGMYEGHLTSSPNPEKGQSSDIPAE

SEQ ID NO: 12 - protein, 93 aa, Arabidopsis thaliana
MGLKEEFEEHAEKVNTLTELPSNEDLLILYGLYKQAKFGPVDTSRPGMFSMKERAKWDAWKAVEGKSSEEAMNDYITKVKQLLEVAASKASTS

SEQ ID NO: 13 - protein, 364 aa, Arabidopsis thaliana
MEVFLEMLLTAVVALLFSFLLAKLVSVATVENDLSSDQPLKPEIGVGVTEDVRFGMKMDARVLESQRNFQVVDENVELVDRFLSEEADRVYEVDEAVTGNAKICGDREAESSAAASSENYVIAEEVILVRGQDEQSDSAEAESISSVSPENVVAEEIKSQGQEEVTELGRSGCVENEESGGDVLVAESEEVRVEKSSNMVEESDAEAENEEKTELTIEEDDDWEGIERSELEKAFAAAVNLLEESGKAEEIGAEAKMELFGLHKIATEGSCREAQPMAVMISARAKWNAWQKLGNMSQEEAMEQYLALVSKEIPGLTKAGHTVGKMSEMETSVGLPPNSGSLEDPTNLVTTGVDESSKNVSGER

SEQ ID NO: 14 - protein, 354 aa, Arabidopsis thaliana
MGDWAQLAQSVILGLIFSYLLAKLISIVVTFKEDNLSLTRHPEESQLEIKPEGVDSRRLDSSCGGFGGEADSLVAEQGSSRSDSVAGDDSEEDDDWEGVESTELDEAFSAATLFVTTAAADRLSQKVPSDVQQQLYGLYKIATEGPCTAPQPSALKMTARAKWQAWQKLGAMPPEEAMEKYIEIVTQLYPTWLDGGVKAGSRGGDDAASNSRGTMGPVFSSLVYDEESENELKIDAIHGFAREGEVENLLKSIESGIPVNARDSEGRTPLHWAIDRGHLNIAKVLVDKNADVNAKDNEGQTPLHYAVVCDREAIAEFLVKQNANTAAKDEDGNSPLDLCESDWPWIRDSAKQAD

SEQ ID NO: 15 - protein, 362 aa, Arabidopsis thaliana
MEVFLEMLLTAVVALLFSFLLAKLVSVATVENDLSSDQPLKPEIGVGVTEDVRFGMKMDARVLESQRNFQVVDENVELVDRFLSEEADRVYEVDEAVTGNAKICGDREAESSAAASSENYVIAEEVILVRGQDEQSDSAEAESISSVSPENVVAEEIKSQGQEEVTELGRSGCVENEESGGDVLVAESEEVRVEKSSNMVEESDAEAENEEKTELTIEEDDDWEGIERSELEKAFAAAVNLLEESGKAEEIGAEAKMELFGLHKIATEGSCREAQPMAVMISARAKWNAWQKLGNMSQEEAMEQYLALVSKEIPGLTKAGHTVGKMSEMETSVGLPPNSGSLEDPTNLVTTGVDESSKNGIP

SEQ ID NO: 16 - protein, 336 aa, Oryza sativa
MGGDWQELAQAAVIGLLFAFLVAKLISTVIAFKEDNLRITRSTPTFPSAADTPAAPAPPPASLDGGHGDTSDGSGSDSDSDWEGVESTELDEDFSAASAFVAASAASGTSVPEQAQLQLYGLYKIATEGPCTAPQPSALKLKARAKWNAWHKLGAMPTEEAMQKYITVVDELFPNWSMGSSTKRKDEDTTVSASSSKGPMGPVFSSLMYEEEDQGNDSELGDIHVSAREGAIDDIAKHLAAGVEVNMRDSEGRTPLHWAVDRGHLNSVEILVNANADVNAQDNEGQTALHYAVLCEREDIAELLVKHHADVQIKDEDGNTVRELCPSSWSFMNLAN

SEQ ID NO: 17 - protein, 91 aa, Oryza sativa
MGLQEDFEQYAEKAKTLPESTSNENKLILYGLYKQATVGDVNTARPGIFAQRDRAKWDAWKAVEGKSKEEAMSDYITKVKQLLEEAAAAAS

SEQ ID NO: 18 - protein, 655 aa, Oryza sativa
MASSGLAYPDRFYAAAAYAGFGAGGATSSSAISRFQNDVALLLYGLYQQATVGPCNVPKPRAWNPVEQSKWTSWHGLGSMPSAEAMRLFVKILEEEDPGWYSRVPEFNPEPVVDIEMHKPKEDPKVILASTNGTSVPEPKTISENGSSVETQDKVVILEGLSAVSVHEEWTPLSVNGQRPKPRYEHGATVVQDKMYIFGGNHNGRYLSDLQALDLKSLTWSKIDAKFQAGSTDSSKSAQVSSCAGHSLISWGNKFFSVAGHTKDPSENITVKEFDPHTCTWSIVKTYGKPPVSRGGQSVTLVGTTLVLFGGEDAKRCLLNDLHILDLETMTWDDVDAIGTPPPRSDHAAACHADRYLLIFGGGSHATCFNDLHVLDLQTMEWSRPKQQGLAPSPRAGHAGATVGENWYIVGGGNNKSGVSETLVLNMSTLTWSVVSSVEGRVPLASEGMTLVHSNYNGDDYLISFGGYNGRYSNEVFALKLTLKSDLQSKTKEHASDGTSSVLEPEVELSHDGKIREIAMDSADSDLKDDANELLVALKAEKEELEAALNREQVQTIQLKEEIAEAEARNAELTKELQTVRGQLAAEQSRCFKLEVDVAELRQKLQSMDALEREVELLRRQKAASEQAALEAKQRQSSSGMWGWLVGTPPDKSES

SEQ ID NO: 19 - protein, 155 aa, Oryza sativa
MGLQEDFEEYAEKVKTLPESTSNEDKLILYGLYKQATVGDVNTSRPGIFAQRDRAKWDAWKAVEGKSKEEAMSDYITKVKQLQEEAAALKAVFRAYLVGEMNIFECHIGRLTRCRRGFRTQMKKQIVYSPGTREMNLLSLIKPSLAHVGYCSTYG

SEQ ID NO: 20 - protein, 354 aa, Arabidopsis thaliana; identical to SEQ ID NO: 14

SEQ ID NO: 21 - protein, 91 aa, Oryza sativa; identical to SEQ ID NO: 17

SEQ ID NO: 22 - protein, 336 aa, Oryza sativa; identical to SEQ ID NO: 16

SEQ ID NO: 23 - protein, 655 aa, Oryza sativa; identical to SEQ ID NO: 18

SEQ ID NO: 24 - protein, 155 aa, Oryza sativa; identical to SEQ ID NO: 19

SEQ ID NO: 25 - protein, 155 aa, Oryza sativa; identical to SEQ ID NO: 19

SEQ ID NO: 26 - protein, 354 aa, Arabidopsis thaliana; identical to SEQ ID NO: 14

SEQ ID NO: 27 - protein, 273 aa, Arabidopsis thaliana
MGDWAQLAQSVILGLIFSYLLAKLISIVVTFKEDNLSLTRHPEESQLEIKPEGVDSRRLDSSCGGFGGEADSLVAEQGSSRSDSVAGDDSEEDDDWEGVESTELDEAFSAATLFVTTAAADRLSQKVPSDVQQQLYGLYKIATEGPCTAPQPSALKMTARAKWQAWQKLGAMPPEEAMEKYIEIVTQLYPTWLDGGVKAGSRGGDDAASNSRGTMGPVFSSLVYDEESENELKIDAIHGFAREGEVENLLKSIESGIPVNARDSEGRTPLHWA

SEQ ID NO: 28 - protein, 669 aa, Arabidopsis thaliana
MAMPRATSGPAYPERFYAAASYVGLDGSDSSAKNVISKFPDDTALLLYALYQQATVGPCNTPKPSAWRPVEQSKWKSWQGLGTMPSIEAMRLFVKILEEDDPGWYSRASNDIPDPVVDVQINQRAKDEPVVENGSTFSETKTISTENGRLAETQDKDVVSEDSNTVSVYNQWTAPQTSGQRPKARYEHGAAVIQDKMYIYGGNHNGRYLGDLHVLDLKSWTWSRVETKVATESQETSTPTLLAPCAGHSLIAWDNKLLSIGGHTKDPSESMQVKVFDPHTITWSMLKTYGKPPVSRGGQSVTMVGKTLVIFGGQDAKRSLLNDLHILDLDTMTWDEIDAVGVSPSPRSDHAAAVHAERFLLIFGGGSHATCFDDLHVLDLQTMEWSRPAQQGDAPTPRAGHAGVTIGENWFIVGGGDNKSGASESVVLNMSTLAWSVVASVQGRVPLASEGLSLVVSSYNGEDVLVAFGGYNGRYNNEINLLKPSHKSTLQTKTLEAPLPGSLSAVNNATTRDIESEVEVSQEGRVREIVMDNVNPGSKVEGNSERIIATIKSEKEELEASLNKERMQTLQLRQELGEAELRNTDLYKELQSVRGQLAAEQSRCFKLEVDVAELRQKLQTLETLQKELELLQRQKAASEQAAMNAKRQGSGGVWGWLAGSPQEKDDDSP

SEQ ID NO: 29 - protein, 648 aa, Arabidopsis thaliana
MAHMVRASSGLSYPERFYAAASYVGLDGSQSSVKQLSSKFSNDTSLLLYTLHQQATLGPCSIPKPSAWNPVEQSKWKSWQGLGTMPSIEAMRLFVKILEEADPGWYPRTSNSVLDPAVHVQINSTKAEPSFESGASFGETKTITSEDGRLTETQDKDVVLEDPDTVSVYNQWTAPRTSGQPPKARYQHGAAVIQDKMYMYGGNHNGRYLGDLHVLDLKNWTWSRVETKVVTGSQETSSPAKLTHCAGHSLIPWDNQLLSIGGHTKDPSESMPVMVFDLHCCSWSILKTYGKPPISRGGQSVTLVGKSLVIFGGQDAKRSLLNDLHILDLDTMTWEEIDAVGSPPTPRSDHAAAVHAERYLLIFGGGSHATCFDDLHVLDLQTMEWSRHTQQGDAPTPRAGHAGVTIGENWYIVGGGDNKSGASKTVVLNMSTLAWSVVTSVQEHVPLASEGLSLVVSSYNGEDIVVAFGGYNGHYNNEVNVLKPSHKSSLKSKIMGASAVPDSFSAVNNATTRDIESEIKVEGKADRIITTLKSEKEEVEASLNKEKIQTLQLKEELAEIDTRNTELYKELQSVRNQLAAEQSRCFKLEVEVAELRQKLQTMETLQKELELLQRQRAVASEQAATMNAKRQSSGGVWGWLAGTPPPKT

SEQ ID NO: 30 - protein, 32 aa, Salmo salar
TRTRKKEKGKSQEDARKEYIALVEELKGKYGV

SEQ ID NO: 31 - protein, 501 aa, Danio rerio
MEGDSNPLYEQRFNAAVKVIQNLPPNGSFQPSHDMMLKFYSYYKQATQGPCNIPRPGFWDPVGKAKWDAWSSLGEMPKEEAMAAYVDDLKLILESMPVSSEVEELLQVIGPFYELVDEKRKITQVSDLSTGFGNLLSSPPKCVTKSIIRTMEMNGNLEGYPIKTAETLKVKSIDLEDREDDDDEDEEGERDEVEEFKEVEKASQPKKRVSAGRPKGPVSNGSISQHKGLSNGTHGSKSDLNRQESEENTEHMNHDGGIVELNGHLNSEKDKEEDVSSSHHVASDSDSEVYCDSVDQFGGEDGSEIHMNRSLEVLEESHSTPSSTGDIRSQDDELLGREEGVQHGGEDGRGSRGGAQRRELPVKRSDSSVVRRGRGSRSPASGSGSAGPQQGSGGDGERWGADGPMTENLNEQIICALARLQDDMQSVLQRLHTLEALTASQARSLALPSDYLTTPANRNKKKPSWWPFDVSLGTVAFAVVWPFVVQWLIRVYVQRRRRRIN

SEQ ID NO: 32 - protein, 494 aa, Danio rerio
MEFDGEDQGSDRLEKTSSENGINTETHALDTQEDHKTCQDNETPQKSWSLDRNWGFTLEELFRLALKFFKEMNGKAFNPTYEENLRLVALHKQITLGPYNPNSCPDIGFFDVLGNDRRKEWLSLGSMAKEDAMEDFVKLLNSCCSLFAPYVTSHKIEKEDQERRQKEEEERLRLEREEQERRRLEEEERRREEEERRAIEEEQRRKEEAQRLQIERQKQQIMAVLNAQTAVQFQQYAKQQYPDNPEQQQLLIRQLQEQHFQQYVQQALRLQQMASQKQQEESTVIQTSEALPAPISNEAVSICNPNSQSNSLDNHKQQKDQKNSHLVVEGETGVVPIVAPSMWTRPQIKEFKEKVLHDEDSVITVGRGEVLTIRVPTHPDGSYLFWEFATDHYDIGFGLHFEWKDLTAPTSSNNGGESTTETKEGTAQAEEEKTEQGRIPLVNEVAPVSRRDSHEEVYAGSHQYPGEGVHLLKFDNSYSLWRPKVVYYRVYYTR

SEQ ID NO: 33 - protein, 362 aa, Arabidopsis thaliana
MEVFLEMLLTAVVALLFSFLLAKLVSVATVENDLSSDQPLKPEIGVGVTEDVRFGMKMDARVLESQRNFQVVDENVELVDRFLSEEADRVYEVDEAVTGNAKICGDREAESSAAASSENYVIAEEVILVRGQDEQSDSAEAESISSVSPENVVAEEIKSQGQEEVTELGRSGCVENEESGGDVLVAESEEVRVEKSSNMVEESDAEAENEEKTELTIEEDDDWEGIERSELEKAFAAAVNLLEESGKAEEIGAEAKMELFGLHKIATEGSCREAQPMAVMISARAKWNAWQKLGNMSQEEAMEQYLALVSKEIPGLTKAGHTVGKVSEMETSVGLPPNSGSLEDPTNLVTTGVDESSKNGIP

SEQ ID NO: 34 - DNA, 2023 bp, Linum usitatissimum
ttcaaaacccgattcccgaggcggccctattgaagatatgggggaagttcgacgagatcgatgtcgggtcgagtgctatggtgatggtgccgtttggggggaggatgagcgagatagccaagactagcattccgttcccacacagagttgggaatttgtaccaaatccaacacttgtcgtattggagcgacgatagggacgcggaaaaacacatccgttggatcagggagttgtacgatgatctcgagccttatgtgtcgaagaatccgaggtatgcttacgtgaactacagggatctcgacatcgggatgaatggaggaggtgaaggggatgagaagggtacttatggtgaggctaaggtgtggggggagaagtactttggggtcaactttgatcggttggttcgggtgaagacgattgttgatcccaataatgtgtttcgaaacgagcagagcattccctcaattccaactcggttataaggatcaatgatcaatgagaattttcctttccaatgtgattacaagttctattgggtcagctttctcaactgctcctattcatttagattaattcataacaactattaatttaccagccttttatccggcccgttggccgatttattttcttaagttttagatgaaatgaaaccgatttagtttttattgagatgagattaatcttaatttgcttgaaatttactcacggttgatgtgatatttggaattaactaaaatgataaatatcggataaaaataaaaatatttaaaataaataacataaacataagaacaataaaataaataaatttaattttaatttatttccttgttttctttctgtatcatacatctcttctcttacttcttaaaggcttttcaattatcacttaattaaatacaatagataaatcgttaattctataacattaacctatacacttgcacggtgaacaatcaatatgataatataataataatataataattcaattattaatctacaattttttaattataaagtttatgcggtcagtttctgcaagctccgagctccttgtcatcgttagtttctgcggtctcaaggtataacgactcggagcgacgagccctttgcttccaatggacgggttgcatttctgccgtcgttgagctcgattggcgtgtcatgctggagtcagagttcctacaaaaaaaccctaaactagagggtgattagggtgaaattagggtgttggcctgggttccattgtccaaagttttagtcaacttaaaaacagacttaaattttatgcttcaaaatagtttatctgttattatattagcgtgtaattagtcttgacaatggggccggacgggtacggattcgggaccccgatccccgcccatagtgtaatggctcaactgccaagtcagcattggaccgaaattattggacacgaagtactaatgtgaaaaactttacatttgttattttctactttaatactatgctattttcaaaatttgaactttaatactatgtttttatatagtttagtatatcttaatttttatgcaaattcatctaattgtattaaactattttcgatccgtagctaattatttcgaaggcaagtcaaagtgttattgtggactatgtgagctaatattgaacctttatctctcccaaccactcaagttaattgaaccaaactcgatcggttgggtttcgagctatttcgagccattgttgttatatgcacgtgagatatcaagattgacccgaacactttattatgataatgtagaaaaagaaaacatattctaagactacatgcatgcaaagtgcaacccctgcatggaaagctgctcaacacgtggcatagactcccgccacgtgtccattccacctcatcacctcacccccaccgttcacctcttattatatcacaacaatcaatcaatcctactcctccatactcgaacaaatccgaccaacttataccaatattcccaaacttgattaatttctcagcaat

SEQ ID NO: 35 - DNA, 1832 bp, Linum usitatissimum
cgaattcaaagaacacaacattgactaacaccaaaaagaaatagagtagtgaaatttggaagattaaaaaatagaaacaaactgattcttagaaagaagagatgattaggtgctttcagttcggtctgtcaggaaatcgagatgttcacttatttacattgtcgattcatctcccaattgtcctggttcctttactgtccgacgcttttttgaatcccagttaattcccatcaagtcttccttcagctgcgtagcactgctagctccaacatggagcgtggagtctactcgttcatggggcatcgcaaaggtttgccttcatgttctgctaccagccagcgcccaccgcctcttggttgtgtggacaattgcggtgaagcgcgcaagttgacatcccatagtctcgacacttcaccatatggatgtttaaaacgtatatcacgagtgcgatctacatgtcccatcacaccacatataaagcaatagtttgggagcttttcatatttgaaacgggcattgacgacttgccctctcgataatttaatctttttttctcttcagctgattgtgtgcatccattcgggctcagaagcacatcaaagggatctctccatcgtagtattgggtcgtgtcgtatgatacgaagcagtcgatgaagtttcctaatgtgcgagctacaggctccgcaaagaacccgcgaggtagatcgtatgctagtacccaaaaatcagtttgtcgtagcggaatcaacactagagactcaccctaatgcatctcatgtgtgatgaacagtttatcatttgtgagtctaggggtcattgtcgatgacccaatgcacattgagcttatgatagaatttgaataggaagcgttttccacccagatcacgaatagctacccctttttcgggcgccaaatttccggcatcctatcttccaccacaacttaaagatgcgatcggtaaggaactcaccgaccacacacatcgaataatcttcggtgaccggttcctgttgatcaagtccctcaatttcctcaacctagtcttcaatcgccgctagcgttatcccccgcatatggactttcatagcgcggagcgtagccggagacgacgagcaagaaggatgagcggcggcagattgcggctaaagaaacgagcttcctgccttgctctatggaggcagatttctgagttgatggtgatggatttgtgatgtggacacttttaatttaagttgattttttagcacttcattcacgtaattaaataaataatttccagtattttatatttatttccttacgttatctaattttttgaaagattaaaactttgatataggcaagatcatgacacgtcgaagttaagtgaatgagactcctaacaaggtaataacaaagcagttcataaaccgaatgaccttgatctttactaagcttgagatcattgaacatataattaaatacgttaatgaaagataagaactttaatataaaaatcattcaaaacgagaaactgataacaaaaacaaagcaaacggccaacaaaataatagacggtggaaggatgatgcagagccatccacccttttttcccagtttccttactgcttacttctctatgcatatcacaagacgcccttgaaacttgttagtcatgcagagcccttactcgccaggtcaccgcaccacgtgttactctatcacttctcctccctttcctttaaagaaccaccacgccacctccctctcacaaacactcataaaaaaaccacctcttgcatttctcccaagttcaaattagttcacagctaagcaagaactcaacaaca

SEQ ID NO: 36 - DNA, 1108 bp, Arabidopsis
ctgcaggaattcgatctctattgattcaaattacgatctgatactgataacgtctagatttttagggttaaagcaatcaatcacctgacgattcaaggtggttggatcatgacgattccagaaaacatcaagcaagctctcaaagctacactctttgggatcatactgaactctaacaacctcgttatgtcccgtagtgccagtacagacatcctcgtaactcggattgtgcacgatgccatgactatacccaacctcggtcttggtcacaccaggaactctctggtaagctagctccactccccagaaacaaccggcgccaaattgcgcgaattgctgacctgaagacggaacatcatcgtcgggtccttgggcgattgcggcggaagatgggtcagcttgggcttgaggacgagacccgaatccgagtctgttgaaaaggttgttcattggggatttgtatacggagattggtcgtcgagaggtttgagggaaaggacaaatgggtttggctctggagaaagagagtgcggctttagagagagaattgagaggtttagagagagatgcggcggcgatgagcggaggagagacgacgaggacctgcattatcaaagcagtgacgtggtgaaatttggaacttttaagaggcagatagatttattatttgtatccattttcttcattgttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaa

SEQ ID NO: 37 - DNA, 1546 bp, bean phaseolin promoter
gaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctac

SEQ ID NO: 38 - DNA, 1145 bp, Brassica napin
aagctttcttcatcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaagtggcataccgttctcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacatacacga

SEQ ID NO: 39 - DNA, 1986 bp, Brassica napus
cccagcattcttctatattccactctattatagagttaaccattggaacaaaaccaattctatagtagagttgttctattttagtggaaataatagaagaaattatagagccacattggagataccctaacggtagtgtttcctttttccgatcaatttattgttagaccaaaagattaaatataatgtaattgaagtgttagaataagaaaagtaattgaagtgttgttgtcaaaaagaaaaaaaaaataatgtttaggaaaagaagtaatcgaagtgttagcaaaaaaaagaaaaaaaagttacaaattcgtatttatgaaaaatggaaaaatggaatgaaattccaaagggactgtgagcagcatgcagccgtacactcgtcatggcggtggaatatgatgaaagcacggctcgtcgcgtccgacacatctctcatgtcacgtgtgtctcagagtcgttttgtgtcgctacgtcttttctctcgattgtcttcttatagttaccatttttgttaacattaatcatttgcatgtccatgattaatcagtctttcgaaactttaacatttctccgagtaggcctaggcgttcgggtacccgctggcattcagatcaggttttttggggtttttgggttttcgaattttcgggtttacgcttctaggtcccatactaaaattttattagtacggatcgggttcggttcaaaattgtattgcatcataaaacccataaagtaatcatatatcgtacggattcgggttatatcggttcggttcggatataaccaaagtaaaaaacaaattttttgaagtaaaagataaagaaaaacatctaaattaaagaaaaattaatctatcacatataaaattgataaaataacaataaaatgttaaatcaagcatgaaaacaaacatcatttataaaaaatatgtattgccttatagagagtagactttttatttcaatgagcaaattataaaatacttatttataactaattgtgtacttaaatcatttattagaattttaatatttattattatatataatattaccacaaatattgaatttaataattggaatacttatatatatttcaaaatatttatattgactattaatttcggatttttcgggttatccgttcgggttcggttaataacacttcgggttcggatattttttgtaccaccctacaagacccgttcgggtatttttacgtttcgggtcggataacggacgggttttttcagttcgggttcggttcggatttcgggttccggattttatatgcccatgcctaaattcgagtgtgaccgttaatccgttatactacgatctaatcaaaacatgtctagatcaaatttgcaatcttattgcatatttttttgtctaacaatattactagaaatctttgtttattaccaacattagtaaaactatcatcttaaccaagttgcaggagcagttcgtttcaaacgtaattgctatagtgatgttattgtaaatttgttatactgatcaaatgtaaagaataatacaatttttatatatatcttgacaaacaaatcagtatatatatacaagaaatatatattttgtcctattacatatgcctatctcaaagttgatgtgtaaagacatgcagttcaataagccatgcaaattgagatgtgtcaaactcccttcgttaatatgtgttttcttacaatgtgaagccaaattaaattttcagaagaagacataaagatagcaactcaaatgaagtgtagattgtacatagtcgactctatatacctggttcttatctcattcaatttatcctcaaaaaaatttatcaacatctatacaaataagttcactataaatagcttcatctaacgcagctgtaagaccagaaaaaccacaacaactaagtaaagagaaaatggctcggctctcatctcttctctctttttccttagcacttttgatctttctc

SEQ ID NO: 40 - DNA, 1943 bp, Brassica napus
ctgcagcccagcattcttctatattccactctattatagagttaaccattggaacaaaaccaattctatagtagagttgttctattttagtggaaataatagaagaaattatagagccacattggagataccctaacggtagtgtttcctttttccgatcaatttattgttagaccaaaagattaaatataatgtaattgaagtgttagaataagaaaaagtaattgaagtgttgttgtcaaaaagaaaaaaaaaataatgtttaggaaaagaagtaatcgaagtgttagcaaaaaaaagaaaaaaaagttacaaattcgtatttatgaaaaatggaaaaatggaatgaaattccaaagggactgtgagcagcatgcagccgtacactcgtcatggcggtggaatatgatgaaagcacggctcgtcgcgtccgacacatctctcatgtcacgtgtgtctcagagtcgttttgtgtcgctacgtcttttctctcgattgtcttcttatagttaccatttttgttaacattaatcatttgcatgtccatgattaatcagtctttcgaaactttaacatttctccgagtaggcctaggcgttcgggtacccgctggcattcagatcaggttttttggggtttttgggttttcgaattttcgggtttacgcttctaggtcccatactaaaattttattagtacggatcgggttcggttcaaaattgtattgcatcataaaacccataaagtaatcatatatcgtacggattcgggttatatcggttcggttcggatataaccaaagtaaaaaacaaattttttgaagtaaaagataaagaaaaacatctaaattaaagaaaaattaatctatcacatataaaattgataaaataacaataaaatgttaaatcaagcatgaaaacaaacatcatttataaaaaatatgtattgccttatagagagtagactttttatttcaatgagcaaattataaaatacttatttataactaattgtgtacttaaatcatttattagaattttaatatttattattatatataatattaccacaaatattgaatttaataattggaatacttatatatatttcaaaatatttatattgactattaatttcggatttttcgggttatccgttcgggttcggttaataacacttcgggttcggatattttttgtaccaccctacaagacccgttcgggtatttttacgtttcgggtcggataacggacgggttttttcagttcgggttcggttcggatttcgggttccggattttatatgcccatgcctaaattcgagtgtgaccgttaatccgttatactacgatctaatcaaaacatgtctagatcaaatttgcaatcttattgcatatttttttgtctaacaatattactagaaatctttgtttattaccaacattagtaaaactatcatcttaaccaagttgcaggagcagttcgtttcaaacgtaattgctatagtgatgttattgtaaatttgttatactgatcaaatgtaaagaataatacaatttttatatatatcttgacaaacaaatcagtatatatatacaagaaatatatattttgtcctattacatatgcctatctcaaagttgatgtgtaaagacatgcagttcaataagccatgcaaattgagatgtgtcaaactcccttcgttaatatgtgttttcttacaatgtgaagccaaattaaattttcagaagaagacataaagatagcaactcaaatgaagtgtagattgtacatagtcgactctatatacctggttcttatctcattcaatttatcctcaaaaaaatttatcaacatctatacaaataagttcactataaatagcttcatctaacgcagctgtaagaccagaaaaaccacaacaactaagtaaagagaccatgg

SEQ ID NO: 41 - DNA, 2035 bp, Linum usitatissimum
ctcaagcatacggacaagggtaaataacatagtcaccagaacataataaacaaaaagtgcagaagcaagataaaaaaattagctatggacattcaggttcatattggaaacatcattatcctagtcttgtgaccatccttcctcctgctctagttgagaggccttgggactaacgagaggtcagttgggatagcagatccttatcctggactagcctttctggtgtttcagagtcttcgtgccgccgtctacatctatctccattaggtctgaagatgactcttcacaccaacgacgtttaaggtctctatcctactcctagcttgcaatacctggcttgcaatacctggagcatcgtgcacgatgattggatactgtggaggaggagtgtttgctgatttagagctcccggttgggtgatttgacttcgatttcagtttaggcttgttgaaatttttcaggttccattgtgaagcctttagagcttgagcttccttccatgttaatgccttgatcgaattctcctagagaaaagggaagtcgatctctgagtattgaaatcgaagtgcacattttttttcaacgtgtccaatcaatccacaaacaaagcagaagacaggtaatctttcatacttatactgacaagtaatagtcttaccgtcatgcataataacgtctcgttccttcaagaggggttttccgacatccataacgacccgaagcctcatgaaagcattagggaagaacttttggttcttcttgtcatggcctttataggtgtcagccgagctcgccaattcccgtccgactggctccgcaaaatattcgaacggcaagttatggacttgcaaccataactccacggtattgagcaggacctattgtgaagactcatctcatggagcttcagaatgtggttgtcagcaaaccaatgaccgaaatccatcacatgacggacgtccagtgggtgagcgaaacgaaacaggaagcgcctatctttcagagtcgtgagctccacaccggattccggcaactacgtgttgggcaggcttcgccgtattagagatatgttgaggcaagacccatctgtgccactcgtacaattacgagagttgttttttttgtgattttcctaagtttctcgttgatggtgagctcatattctacatcgtatggtctctcaacgtcgtttcctgtcatctgatatcccgtcatttgcatccacgtgcgccgcctcccgtgccaagtccctaggtgtcatgcacgccaaattggtggtggtgcgggctgccctgtgcttcttaccgatgggtggaggttgagtttgggggtctccgcggcgatggtagtgggttgacggtttggtgtgggttgacggcattgatcaatttacttcttgcttcaaattctttggcagaaaacaattcattagattagaactggaaaccagagtgatgagacggattaagtcagattccaacagagttacatctcttaagaaataatgtaacccctttagactttatatatttgcaattaaaaaaataatttaacttttagactttatatatagttttaataactaagtttaaccactctattatttatatcgaaactatttgtatgtctcccctctaaataaacttggtattgtgtttacagaacctataatcaaataatcaatactcaactgaagtttgtgcagttaattgaagggattaacggccaaaatgcactagtattatcaaccgaatagattcacactagatggccatttccatcaatatcatcgccgttcttcttctgtccacatatcccctctgaaacttgagagacacctgcacttcattgtccttattacgtgttacaaaatgaaacccatgcatccatgcaaactgaagaatggcgcaagaacccttcccctccatttcttatgtggcgaccatccatttcaccatctcccgctataaaacacccccatcacttcacctagaacatcatcactacttgcttatccatccaaaagatacccacc

SEQ ID NO: 42 - DNA, 766 bp, Cauliflower mosaic virus
gagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatccgtcctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcagatccgtcgacgtcgaggaattccccggatcgtttc

SEQ ID NO: 43 - DNA, 1245 bp, rice
tagctagcatactcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgatttggaaatgcatatctgtatttgagtcggtttttaagttcgttgcttttgtaaatacagagggatttgtataagaaatatctttaaaaaacccatatgctaatttgacataatttttgagaaaaatatatattcaggcgaattccacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacccccggcactatcaccgtgagttgtccgcaccaccgcacgtctcgcagccaaaaaaaaaaaaagaaagaaaaaaaagaaaaagaaaaacagcaggtgggtccgggtcgtgggggccggaaaagcgaggaggatcgcgagcagcgacgaggcccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcccctctcctctttctttctccgttttttttttcgtctcggtctcgatctttggccttggtagtttgggtgggcgagagcggcttcgtcgcccagatcggtgcgcgggaggggcgggatctcgcggctggcgtctccgggcgtgagtcggcccggatcctcgcggggaatggggctctcggatgtagatcttctttctttcttctttttgtggtagaatttgaatccctcagcattgttcatcggtagtttttcttttcatgatttgtgacaaatgcagcctcgtgcggagcttttttgtaggtagaag

SEQ ID NO: 44 - DNA, 1649 bp, corn
gatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctataatttctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcac

SEQ ID NO: 45 - DNA, 998 bp, parsley
gaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccacc

SEQ ID NO: 46 - protein, 173 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTT

SEQ ID NO: 47 - protein, 173 aa, Arabidopsis thaliana; identical to SEQ ID NO: 46

SEQ ID NO: 48 - protein, 173 aa, Arabidopsis thaliana; identical to SEQ ID NO: 46

SEQ ID NO: 49 - protein, 199 aa, Arabidopsis thaliana
MADTHRVDRTDRHFQFQSPYEGGRGQGQYEGDRGYGGGGYKSMMPESGPSSTQVLSLLIGVPVVGSLLALAGLLLAGSVIGLMVALPLFLLFSPVIVPAGLTIGLAMTGFLASGMFGLTGLSSISWVMNYLRGTKRTVPEQLEYAKRRMADAVGYAGQKGKEMGQHVQNKAQDVKQYDISKPHDTTTKGHETQGGTTAA

SEQ ID NO: 50 - protein, 191 aa, Arabidopsis thaliana
MANVDRDRRVHVDRTDKRVHQPNYEDDVGFGGYGGYGAGSDYKSRGPSTNQILALIAGVPIGGTLLTLAGLTLAGSVIGLLVSIPLFLLFSPVIVPAALTIGLAVTGILASGLFGLTGLSSVSWVLNYLRGTSDTVPEQLDYAKRRMADAVGYAGMKGKEMGQYVQDKAHEARETEFMTETHEPGKARRGS

SEQ ID NO: 51 - protein, 175 aa, Brassica napus
RRDQYPRDRDQYSMIGRDRDKYSMIGRDRDQYNMYGRDYSKSRQIAKAVTAVTAGGSLLVLSSLTLVGTVIALTVATPLLVIFSPILVPALITVALLITGFLSSGGFGIAAITVFSWIYKYATGEHPQGSDKLDSARMKLGGKVQDMKDRAQYYGQQQTGGEDDRDRTRGTQHTT

SEQ ID NO: 52 - protein, 195 aa, Brassica napus
MTDTARTHHDITSRDQYPRDRDQYSMIGRDRDQYSMMGRDRDQYNMYGRDYSKSRQIAKAVTAVTAGGSLLVLSSLTLVGTVIALTVATPLLVIFSPILVPALITVAMLITGFLSSGGFGIAAITVFSWIYKYATGEHPQGSDKLDSARMKLGSKAQDLKDRAQYYGQQHTGGYGQQHTGGEHDRDRTRGTQHTT

SEQ ID NO: 53 - protein, 195 aa, Brassica napus; identical to SEQ ID NO: 52

SEQ ID NO: 54 - protein, 183 aa, Brassica napus
PARTHHDITTRDQYPLISRDRDQYGMIGRDQYNMSGQNYSKSRQIAKATTAVTAGDSLLVLSSLTLVGTVIALIVATPLLVIFSPILVPALITVALLITGFLSSGAFGIAAITVFSWIYKYATGEHPQGSDKLDSARMKLGSKAQDMKDRAYYYGQQHTGEEHDRDRDHRTDRDRTRGTQHTT

SEQ ID NO: 55 - protein, 183 aa, Brassica napus; identical to SEQ ID NO: 54

SEQ ID NO: 56 - protein, 195 aa, Brassica napus; identical to SEQ ID NO: 52

SEQ ID NO: 57 - protein, 175 aa, Brassica napus; identical to SEQ ID NO: 51

SEQ ID NO: 58 - protein, 195 aa, Brassica napus; identical to SEQ ID NO: 52

SEQ ID NO: 59 - protein, 165 aa, Brassica napus
MGILRKKKHERKPSFKSVLTAILATHAATFLLLIAGVSLAGTAAAFIATMPLFVVFSPILVPAGITTGLLTTGLAAAGGAGATAVTIILWLYKRATGKAPPKVLEKVLKKIIPGAAAAPAAAPGAAPAAAPAAAPAVAPAAAPAAAPAPKPAAPPAPKPAAAPSI

SEQ ID NO: 60 - protein, 375 aa, Brassica napus
MKEEIQNETAQTQLQREGRMFSFLFPVIEVIKVVMASVASVVFLGFGGVTLACSAVALAVSTPLFIIFSPILVPATIATTLLATGLGAGTTLGVTGMGLLMRLIKHPGKEGAASAPAAQPSFLSLLEMPNFIKSKMLERLIHIPGVGKKSEGRGESKGKKGKKGKSEHGRGKHEGEGKSKGRKGHRMGVNPENNPPPAGAPPTGSPPAAPAAPEAPAAPAAPAAPAAPAAPAAPAAPEDPAAPAAPEAPATPAAPPAPAAAPAPAAPAAPPAPAAPPRPPSFLSLLEMPSFIKSKLIEALINIPGFGKKSNDRGKSKGGKKSKGKGKSNGRGKHEGEGKSKSRKSKSRGKDKEKSKGKGIFGRSSRKGSSDDESS

SEQ ID NO: 61 - protein, 168 aa, Daucus carota
  misc_feature (55)..(55): Xaa can be any naturally occurring amino acid
NKFTLSNLISVDFMAMYQSPQFTRHHDALQPAALNSSGQGHHSSHHRRSVMLLSXLTLVATVIGLVIATPVMVIFSPVLVPAGLPAAPARRVSHGGGAGGHRAFVLFWMYRYTAGKHPIGANQLDFAATRLRMRKEKGXIWGMSRFRLFRGVEEVVRRXGNDEGFLAV

SEQ ID NO: 62 - protein, 168 aa, Daucus carota; identical to SEQ ID NO: 61
  misc_feature (55)..(55): Xaa can be any naturally occurring amino acid

SEQ ID NO: 63 - protein, 187 aa, Zea mays
MADRDRSGIYGGAHATYGQQQQQGGGGRPMGEQVKKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEARRRMAEAAAQAGHKTAQAGQAIQGRAQEAGTGGGAGAGAGGGGRASS

SEQ ID NO: 64 - protein, 187 aa, Zea mays; identical to SEQ ID NO: 63

SEQ ID NO: 65 - protein, 156 aa, Zea mays
MADHHRGATGGGGGYGDLQRGGGMHGEAQQQQKQGAMMTALKAATAATFGGSMLVLSGLILAGTVIALTVATPVLVIFSPVLVPAAIALALMAAGFVTSGGLGVAALSVFSWMYKYLTGKHPPAADQLDHAKARLASKARDVKDAAQHRIDQAQGS

SEQ ID NO: 66 - protein, 175 aa, Zea mays
MADRDRSGIYGGGAYGQQQGRPPMGEQVKGMIHDKGPTASQALTVATLFPLGGLLLVLSGLALAASTVGLAVATPVFLLFSPVLVPAALLIGTAVAGFLTSGALGLGGLSSLTCLANTARQAFQRTPDYVEEARRRMAEAAAHAGHKTAQAGHGIQSKAQEAGAGTGAGGGRTSS

SEQ ID NO: 67 - protein, 156 aa, Zea mays; identical to SEQ ID NO: 65

SEQ ID NO: 68 - protein, 187 aa, Zea mays; identical to SEQ ID NO: 63

SEQ ID NO: 69 - protein, 156 aa, Zea mays; identical to SEQ ID NO: 65

SEQ ID NO: 70 - protein, 175 aa, Zea mays; identical to SEQ ID NO: 66

SEQ ID NO: 71 - protein, 217 aa, Oryza sativa
MALVLFASSSSCKRPVTHNILCGTRAKGSPAAAAAVGAATELQKHVAFFDSNHDGIISFSETYEGFRALGFGVVTSRFSATVINGALGTKTRPENATASRFSIYIENIHKGVHGSDTGAFDSEGRFVNEKFDEIFTKHAKTVPDGLTAAELDEMLRANREPKDYKGWVGASTEWETTFKLGKDKDGFLRKDTVRTVYDGSFFSKVASKKKGPSANQA

SEQ ID NO: 72 - protein, 174 aa, Arabidopsis thaliana
MFFCFCFCESKKGLCMETYLWDYVVYVGGKLDKEKMTALEKHVSFFDRNKDGTVYPWETYQGFRALGTGRLLAAFVAIFINMGLSKKTRPGKGFSPLFPIDVKNSHLCMHGSDTDVYDDDGRFVESKFEEIFNKHARTHKDALTAEEIQKMLKTNRDPFDITGWFVVSELFQTN

SEQ ID NO: 73 - protein, 192 aa, Arabidopsis thaliana
MASSISAAEVKVVPEEYNFLQKHVAFFDRNKDGIVYPSETFQGFRAIGCGYLLSTFAAVFINISLSSKTRPGKGFSFSFPIEVKNVRLGIHSSDSGVYDKDGRFVASKFEEIFAKHAHTHRDALTSKELKELLKANREPNDCKGGILAFGEWKVLYNLCKDKSGLLHKEIVRAVYDGSLFEQLEKQRSSKTP

SEQ ID NO: 74 - protein, 195 aa, Arabidopsis thaliana
MASSISTGVKFVPEEDNFLQRHVAFFDRNKDGIVYPSETFQGFRAIGCGYLLSAVASVFINIGLSSKTRPGKGFSIWFPIEVKNIHLAKHGSDSGVYDKDGRFVASKFEEIFTKHAHTHRDALTNEELKQLLKANKEPNDRKGWLAGYTEWKVLHYLCKDKNGLLHKDTVRAAYDGSLFEKLEKQRSSKTSKKHP

SEQ ID NO: 75 - protein, 246 aa, Hordeum vulgare
MAGEDATRAATEEELSSVAEAAPVTAQRPVRSDLEKYIPKPYLARALVAPDVYHPQGSKERGHEHRHRSVLQQHVAFFDMDGDGVIYPWETYQGLRALGFNMIVSFVIVIIIHATLSYTTLPSWVPSLLFPFYIDNIHRAKHGSDTATYDTEGRYMPVNFENIFSKNARSSPDKLTFREIWTMTDDQRQANDPFGWVASKAEWILLYMLAKDEEGNLPREAIRRCFDGSLFEFIADERRQAHGKQY

SEQ ID NO: 76 - protein, 246 aa, Hordeum vulgare; identical to SEQ ID NO: 75

SEQ ID NO: 77 - protein, 301 aa, Hordeum vulgare
MATKARKVEVRDASRAEGKGDAADVHVLREAMRADGKGDHDTAGGANRADGHGDAGGRVGDSRGVDGKDSLKMVALQAPVTVERPVRGDLEEHVPKPYLARALAAPDMYHPEGTTTDDHQHHNMSVLQQHVAFFDRDNNGIIYPWETYDGCRAVGFNVFMSAFIAFLVNLVMSYPTLPGWLPNPLFPIYVHNIHKSKHGSDSGTYDKEGRFMPVNFENIFSKYARTYPDRLSYREMWRMTEGCREVFDFFGWVAMKLEWSILYALARDDEGYLSREAIRRMYDGSLFEYMERQRMEHVKMS

SEQ ID NO: 78 - protein, 301 aa, Hordeum vulgare; identical to SEQ ID NO: 77

SEQ ID NO: 79 - protein, 118 aa, Arabidopsis thaliana
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYK

SEQ ID NO: 80 - protein, 187 aa, Brassica napus
MADTARTHHDVTSRDQYPRDRDQYSMIGRDRDQYSMMGRDRDQYNMYGRDYSKSRQIAKAVTAVTAGGSLLVLSSLTLVGTVIALTVATPLLVIFSPILVPALITVALLITGFLSSGGFAIAAITVFSWIYKYATGEHPQGSDKLDSARMKLGTKAQDIKDRAQYYGQQHTGGEHDRDRTRGGQHTT

SEQ ID NO: 81 - DNA, 748 bp, Arabidopsis thaliana
taccatggggtcaaagacggagatgatggagagagacgcaatggctacggtggctccctatgcgccggtcacttaccatcgccgtgctcgtgttgacttggatgatagacttcctaaaccttatatgccaagagcattgcaagcaccagacagagaacacccgtacggaactccaggccataagaattacggacttagtgttcttcaacagcatgtctccttcttcgatatcgatgataatggcatcatttacccttgggagacctactctggactgcgaatgcttggtttcaatatcattgggtcgcttataatagccgctgttatcaacctgacccttagctatgccactcttccggggtggttaccttcacctttcttccctatatacatacacaacatacacaagtcaaagcatggaagtgattcaaaaacatatgacaatgaaggaaggtttatgccggtgaatcttgagttgatatttagcaaatatgcgaaaaccttgccagacaagttgagtcttggagaactatgggagatgacagaaggaaaccgtgacgcttgggacatttttggatggatcgcaggcaaaatagagtggggactgttgtacttgctagcaagggatgaagaagggtttttgtcaaaagaagctattaggcggtgtttcgatggaagcttgttcgagtactgtgccaaaatctacgctggtatcagtgaagacaagacagcatactacgccatggat

SEQ ID NO: 82 - DNA, 738 bp, Arabidopsis thaliana
atggggtcaaagacggagatgatggagagagacgcaatggctacggtggctccctatgcgccggtcacttaccaccgccgtgctcgtgttgacttggatgatagacttcctaaaccttatatgccaagagcattgcaagcaccagacagagaacacccgtacggaactccaggccataagaattacggacttagtgttcttcaacagcatgtctccttcttcgatatcgatgataatggcatcatttacccttgggagacctactctggactgcgaatgcttggtttcaatatcattgggtcgcttataatagccgctgttatcaacctgacccttagctatgccactcttccggggtggttaccttcacctttcttccctatatacatacacaacatacacaagtcaaagcatggaagtgattcaaaaacatatgacaatgaaggaaggtttatgccggtgaatcttgagttgatatttagcaaatatgcgaaaaccttgccagacaagttgagtcttggagaactatgggagatgacagaaggaaaccgtgacgcttgggacatttttggatggatcgcaggcaaaatagagtggggactgttgtacttgctagcaagggatgaagaagggtttttgtcaaaagaagctattaggcggtgtttcgatggaagcttgttcgagtactgtgccaaaatctacgctggtatcagtgaagacaagacagcatactactaa

SEQ ID NO: 83 - DNA, 1047 bp, Sesamum indicum
atggatctaatccacactttcctcaacttaatagctccccctttcaccttcttcttccttctctttttcttgccacccttccagattttcaagttcttcctttcaatcttgggcacccttttcagcgaggatgtcgctggaaaagtcgtcgtcatcaccggcgcctcctccggcatcggcgaaagtcttgcttacgagtatgctaagagaggggcgtgcttggtgcttgctgcaagaagggaaaggagtcttcaagaagtggccgaaagggcgcgcgatttggggtcgccggacgtcgtggtggtccgggccgatgtttcgaaggcggaggactgcaggaaggttgttgatcagactatgaatcgctttggaagattggatcacctggtcaataacgctggaattatgtcagtttcaatgctggaagaagttgaagatattactggttacagagaaactatggatatcaacttctggggctatgtgtatatgacccgatttgccgccccataccttaggaatagcagaggccgaattgttgtactttcttcatccagttcttggatgcctactccgaggatgagtttttacaatgcaagcaaagcggcgatttcacaattttttgagacactgcgggtggaattcggccccgatataggcataacccttgtgactccaggattcatagaatctgaacttacccaaggcaaattctacaatgctggcgaacgtgtaattgatcaggacatgagagatgtacaagtgagcacgactccaatcctgagggtggaaagtgcggcaaggtcaatcgtgaggagcgcgatccgtggagaaagatacgtgacagagccggcctggtttagggttacttattggtggaagctattctgccctgaggtgatggagtgggtatttagactgatgtacttggccagcccgggtgagccggagaaggaaacgtttggcaagaaggttttggattacacaggagtgaagtccttgctttacccggaaaccgtgcaagttccggagcccaagaatgattaa

SEQ ID NO: 84 - DNA, 190 bp, Artificial Sequence
  Synthetic
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcag

SEQ ID NO: 85 - DNA, 756 bp, Arabidopsis thaliana
atggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtagaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacact

SEQ ID NO: 86 - DNA, 279 bp, Brassica napus
atgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagcttga

SEQ ID NO: 87 - DNA, 291 bp, Brassica napus
atgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagctaaggacgaactctga

SEQ ID NO: 88 - DNA, 237 bp, Papaver somniferum
atgatgtgcagaagcttaacattacgtttcttcttattcattgttttattacaaacatgcgtacgaggtggtgatgttaatgataatctcctctcgtcatgtttaaactcccatggtgttcacaacttcaccacgctatcaaccgatacaaattccgactacttcaaactgctgcatgcatccatgcagaacccgttgttcgcgaagcctacggtatcgaaaccgtcgtttattgtc

SEQ ID NO: 89 - DNA, 411 bp, Artificial Sequence
  Synthetic
atggcggatactgctagaggaacccatcacgatatcatcggcagagaccagtacccgatgatgggccgagaccgagaccagtaccagatgtccggacgaggatctgactactccaagtctagacagattgctaaaggcgccggaactgtcatagctttgactgttgcaacacctctgctcgttatcttcagcccaatccttgtcccggctctcatcacagttgcactcctcatcaccggtatttttaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacact

SEQ ID NO: 90 - DNA, 70 bp, Tobacco
atgaacttccttaagtctttccctttctacgctttcctttgtttcggtcaatacttcgttgctgttacgc

SEQ ID NO: 91 - DNA, 753 bp, Artificial Sequence
  Synthetic
atgatagacattgtgatgacacagtctccatcctccctggctatgtcagtgggacagcgggtcactatgcgctgcaagtccagtcagagccttttaaaaagtaccaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattataacactcctcccacgttcggtgctgggaccaagctggagcttaagcggtctccgaacggtgcttctcatagcggttctgcaccaggcactagctctgcatctggatctcaggtgcacctgcagcagtctggagctgagctgatgaagcctggggcctcaatgaagatatcctgcaaggctactggctacacattcagtagctactggatagagtgggtaaagcagaggcctggacatggccttgagtggattggagagattttacctggcagtggtagtactacctacaatgagaagttcaagggcaaggccacattcactgcagatacatcctccaacacagcctacatgcaactcagcagcctgacatctgaggactctgccgtctattactgtgcaagattggatgttgactcctggggccaaggcaccactctcacagtctcgagtgcc

SEQ ID NO: 92 - DNA, 1226 bp, Bean
gcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaa

SEQ ID NO: 93 - DNA, 552 bp, Artificial Sequence
  Pat gene
atgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctga

SEQ ID NO: 94 - DNA, 490 bp, Parsley
gtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaa

SEQ ID NO: 95 - DNA, 311 bp, Artificial Sequence
  Synthetic
cagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 96 - DNA, 6371 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctactatggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtagaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactaccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagcttgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 97 - DNA, 6408 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagccatggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtagaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactacttaagttaccccactgatgtcatcgtctagatttaaatgcaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 98 - DNA, 6608 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaccatgatgtgcagaagcttaacattacgtttcttcttattcattgttttattacaaacatgcgtacgaggtggtgatgttaatgataatctcctctcgtcatgtttaaactcccatggtgttcacaacttcaccacgctatcaaccgatacaaattccgactacttcaaactgctgcatgcatccatgcagaacccgttgttcgcgaagcctacggtatcgaaaccgtcgtttattgtcatggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtagaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactaccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagcttgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 99 - DNA, 6039 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaccatggcggatactgctagaggaacccatcacgatatcatcggcagagaccagtacccgatgatgggccgagaccgagaccagtaccagatgtccggacgaggatctgactactccaagtctagacagattgctaaaggcgccggaactgtcatagctttgactgttgcaacacctctgctcgttatcttcagcccaatccttgtcccggctctcatcacagttgcactcctcatcaccggtatttttaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggtcaacacactactagttcagtcatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagcttgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagctagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 100 - DNA, 5699 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaccatgaacttccttaagtctttccctttctacgctttcctttgtttcggtcaatacttcgttgctgttacgcatgccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagctaaggacgaactctgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 101 - DNA, 6452 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaccatgaacttccttaagtctttccctttctacgctttcctttgtttcggtcaatacttcgttgctgttacgcatgccatgatagacattgtgatgacacagtctccatcctccctggctatgtcagtgggacagcgggtcactatgcgctgcaagtccagtcagagccttttaaaaagtaccaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattataacactcctcccacgttcggtgctgggaccaagctggagcttaagcggtctccgaacggtgcttctcatagcggttctgcaccaggcactagctctgcatctggatctcaggtgcacctgcagcagtctggagctgagctgatgaagcctggggcctcaatgaagatatcctgcaaggctactggctacacattcagtagctactggatagagtgggtaaagcagaggcctggacatggccttgagtggattggagagattttacctggcagtggtagtactacctacaatgagaagttcaagggcaaggccacattcactgcagatacatcctccaacacagcctacatgcaactcagcagcctgacatctgaggactctgccgtctattactgtgcaagattggatgttgactcctggggccaaggcaccactctcacagtctcgagtgccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagctaaggacgaactctgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 102 - DNA, 5612 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagcttgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 103 - DNA, 6365 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggaattcattgtactcccagtatcattatagtgaaagttttggctctctcgccggtggttttttacctctatttaaaggggttttccacctaaaaattctggtatcattctcactttacttgttactttaatttctcataatctttggttgaaattatcacgcttccgcacacgatatccctacaaatttattatttgttaaacattttcaaaccgcataaaattttatgaagtcccgtctatctttaatgtagtctaacattttcatattgaaatatataatttacttaattttagcgttggtagaaagcataaagatttattcttattcttcttcatataaatgtttaatatacaatataaacaaattctttaccttaagaaggatttcccattttatattttaaaaatatatttatcaaatatttttcaaccacgtaaatctcataataataagttgtttcaaaagtaataaaatttaactccataatttttttattcgactgatcttaaagcaacacccagtgacacaactagccatttttttctttgaataaaaaaatccaattatcattgtattttttttatacaatgaaaatttcaccaaacaatcatttgtggtatttctgaagcaagtcatgttatgcaaaattctataattcccatttgacactacggaagtaactgaagatctgcttttacatgcgagacacatcttctaaagtaattttaataatagttactatattcaagatttcatatatcaaatactcaatattacttctaaaaaattaattagatataattaaaatattacttttttaattttaagtttaattgttgaatttgtgactattgatttattattctactatgtttaaattgttttatagatagtttaaagtaaatataagtaatgtagtagagtgttagagtgttaccctaaaccataaactataacatttatggtggactaattttcatatatttcttattgcttttaccttttcttggtatgtaagtccgtaactagaattacagtgggttgccatgacactctgtggtcttttggttcatgcatgggtcttgcgcaagaaaaagacaaagaacaaagaaaaaagacaaaacagagagacaaaacgcaatcacacaaccaactcaaattagtcactggctgatcaagatcgccgcgtccatgtatgtctaaatgccatgcaaagcaacacgtgcttaacatgcactttaaatggctcacccatctcaacccacacacaaacacattgcctttttcttcatcatcaccacaaccacctgtatatattcattctcttccgccacctcaatttcttcacttcaacacacgtcaacctgcatatgcgtgtcatcccatgcccaaatctccatgcatgttccaaccaccttctctcttatataatacctataaatacctctaatatcactcacttctttcatcatccatccatccagagtactactactctactactataataccccaacccaactcatattcaatactactctaccatgatagacattgtgatgacacagtctccatcctccctggctatgtcagtgggacagcgggtcactatgcgctgcaagtccagtcagagccttttaaaaagtaccaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattataacactcctcccacgttcggtgctgggaccaagctggagcttaagcggtctccgaacggtgcttctcatagcggttctgcaccaggcactagctctgcatctggatctcaggtgcacctgcagcagtctggagctgagctgatgaagcctggggcctcaatgaagatatcctgcaaggctactggctacacattcagtagctactggatagagtgggtaaagcagaggcctggacatggccttgagtggattggagagattttacctggcagtggtagtactacctacaatgagaagttcaagggcaaggccacattcactgcagatacatcctccaacacagcctacatgcaactcagcagcctgacatctgaggactctgccgtctattactgtgcaagattggatgttgactcctggggccaaggcaccactctcacagtctcgagtgccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagcttgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 104 - DNA, 4833 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatccgtcctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcagatccgtcgacgtcgaggaattccccggatcgtttcccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagcttgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 105 - DNA, 5629 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatccgtcctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcagatccgtcgacgtcgaggaattccccggatcgtttcccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagccatggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtagaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactacttaagttaccccactgatgtcatcgtctagatttaaatgcaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 106 - DNA, 4845 bp, Artificial Sequence
  DNA Construct
ttgatcccgaggggaaccctgtggttggcttgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccctgcaggagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatccgtcctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcagatccgtcgacgtcgaggaattccccggatcgtttcccatgggtttgaaggaggactttgaggagcacgctgagaaagtcaagaagctcaccgcgagcccatctaacgaggacttgctcatcctctacggtctctacaagcaagccaccgttgggccagtgaccaccagtcgtcctgggatgttcagcatgaaggaaagagccaagtgggacgcttggaaggccgttgaagggaaatcaacggacgaagccatgagtgactacatcactaaggtgaagcaactccttgaagcagaggcttcctccgcttcagctaaggacgaactctgaagcttaaataagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaactttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaattttttttttttatcagcaaagaaataaaattaaattaagaaggacaatggtgtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacccttatacaaaaccccaaaaacaagtttcctagcaccctaccaactaaggtaccgaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcatttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagccaaggctacgggcgatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggttccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgaccgaatgagttccaagatggtttgtgacgaagttagttggttgtttttatggaactttgtttaagctagcttgtaatgtggaaagaacgtgtggctttgtggtttttaaatgttggtgaataaagatgtttcctttggattaactagtatttttcctattggtttcatggttttagcacacaacattttaaatatgctgttagatgatatgctgcctgctttattatttacttacccctcaccttcagtttcaaagttgttgcaatgactctgtgtagtttaagatcgagtgaaagtagattttgtctatatttattaggggtatttgatatgctaatggtaaacatggtttatgacagcgtacttttttggttatggtgttgacgtttccttttaaacattatagtagcgtccttggtctgtgttcattggttgaacaaaggcacactcacttggagatgccgtctccactgatatttgaacaaagaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgt

SEQ ID NO: 107 - DNA, 1678 bp, Bean
gtagacaaaatcccatcttttcctacataattcttctacagttaaccttcaaatcatattttcattattcacaaatatcattcatacgaataaatatatatttttttcacatacaattatgataatatattaaaaagtgaactttaaatttaatttaatcttatcttataaaatgagatttctacctacgattaataaaaataactttgatatcatattaaaaaataaactttaaacctaactcaactttataaaaccaataaaatttacactcagttatgaattataaaatgaaatagtttttaggtgacgtggaatctccatccgattaatcaatatttgggtgatgttattgttattatagacatgccaaataatttacaatatatagattcagttaaatcaattcagcttgtctccttgactaataaaaaaaaactttagactattattcagatttacactatccctcaaagtgaatttcattcatggcaccatttatataatcaacaattttaaaaatatgcaaatttgtaccagtaaatgctttaatgtctgataaacacaaaaaaaaaaaaattcatatttttttcttattaaataaagaagttcattgtaagagaaattaggatccttcaatagaaaatgtgttacaaaggggcaacagttaacaaaacaaatttatgtttcatttgagattaaggaaggtaaggaagaaaaaagattaaaaaaaatgtccttatctctttataagagacttaaacttttaatataataattgtaattaggttttctagtcatgagcaccactcagagacaagatttcaagaaaacaattttgttaaacatcttattagaaacttttagttaagtcttgaagttagaattaaacaaaaaaaagtacacacgagaaacacaataaacccactaccgtcaggttatcataaggatttgatatcattaaatataacacacacaaaaatacatctaattataacaatatatgttatacatatatttttgtaaaaacttagagtttttattctaatacatgattagagtttatagaaatacaaatatttaaaaaatataattttaaaaaaacattctaaagtcattcagatcctctcacacctgttagtcatgtatgtagtacaatcattgtagttcacaacagagtaaaataaataaggataaactagggaatatatataatatatacaattaaataaaattagaatttttgattccccacatgacacaactcaccatgcacgctgccacctcagctccctcctctccacacatgtctcatgtcactttcgacttcactatgacacaactcgccatgcatgttgccacgtgagctccttcctcttcccatgatgacaccactgggcatgcatgctgccacctcagctcccgagcctactggccatgcacactgccacctcagcactcctctcacttcccattgctacctgccaaaccgcttctctccataaatatctatttaaatttaaactaattatttcatatacttttttgatgacgtggatgcattgccatcgttgtttaataattgttaataaaatgaaagaaaaaagttggaaagattttgcatttgttgttgtataaatagagaagagagtgatggttaatgcatgaatgcatgatc

SEQ ID NO: 108 - DNA, 279 bp, Brassica napus; identical to SEQ ID NO: 86

(19) United States
(12) Patent Application Publication
(10) Pub. No.: US 2012/0255066 A1
(43) Pub. Date: Oct. 4, 2012

(54) TRANSGENIC EXPRESSION OF ACYL-CO-A BINDING PROTEINS IN PLANTS

(75) Inventors: Maurice M. Moloney, Calgary (CA); Randall Joseph Weselake, Edmonton (CA); Cory L. Nykiforuk, Calgary (CA); Olga Petrivna Yurchenko, Edmonton (CA)

(73) Assignees: THE GOVERNORS OF THE UNIVERSITY OF ALBERTA, Edmonton, AB (CA); SEMBIOSYS GENETICS INC., Calgary, AB (CA)

(21) Appl. No.: 12/531,330

(22) PCT Filed: Mar. 17, 2008
(86) PCT No.: PCT/CA2008/000500

§ 371 Date: Feb. 24, 2012

Related U.S. Application Data
(60) Provisional application No. 60/895,197, filed on Mar. 16, 2007.
(60) Provisional application No. 60/942,120, filed on Jun. 5, 2007.

(51) Int. Cl. C12N 15/82; A01H 5/10; A01H 5/00; C12N 5/10

(52) U.S. Cl. 800/281; 435/320.1; 435/419; 800/298; 800/306; 800/312; 800/314; 800/313

(57) ABSTRACT

Disclosed are methods for modification of fatty acid composition and/or seed oil content in plants. In particular are methods to over-express acyl-CoA binding proteins (ACBPs) within the cells of developing seeds are provided. Over-expressing ACBPs under the control of a seed preferred promoter increases polyunsaturated fatty acid (PUFA) levels as compared to wild-type controls.

### FIELD OF THE INVENTION

The present disclosure relates to methods of enhancing or modifying oil production in plants or plant seeds.

### BACKGROUND

Edible fats and oils are the most condensed source of energy in the human diet, with 70-80% of lipids consumed originating from plants, mainly from seeds and mesocarp tissues of fruits (Ohlrogge et al. 2004, Proceedings of the 4^{th} International Crop Sciences Congress, October 2004, Brisbane, Australia. www.cropscience.org.au). The major component of plant oils is triacylglycerol (“TAG”), which consists of a glycerol molecule esterified with three fatty acid (“FA”) moieties (Weselake, 2002, Pp. 27-56, In: Kuo, T. M. and Gardner, H. W. (eds). Lipid Biotechnology. Marcel Dekker, New York). FA composition is one of the most important characteristics of edible oils, affecting both the physical and nutritional properties of the oil. There are a variety of FAs in seed oils that differ in carbon chain length, degree of unsaturation and positional distribution on the glycerol backbone. Attempts to modify FA composition of seed oils have been successful to varying degrees by both conventional plant breeding and genetic engineering (Downey and Craig, 1964, J. Am. Oil Chem. Soc. 41:475-478; Cole et al., 1998, Lipids 100:177-181; Gunstone and Pollard, 2001, Pp 155-184, In: Gunstone, F. D. (ed.) Structured and Modified Lipids. Marcel Dekker, New York).

In developing seeds of plants, FA synthesis occurs in the plastid through the catalytic action of acetyl-CoA carboxylase and the FA synthase complex (Harwood, 1996, Biochimica et Biophysica Acta 1301:7-56). The first FA desaturation step also takes place in plastids through the enzymatic action of acyl-ACP-desaturase, resulting in production of monounsaturated FA (MUFA) (Jaworski, 1987, The Biochemistry of Plants 9:159-173). Newly synthesized FAs are released from the FA synthase complex by acyl-ACP hydrolase (thioesterase). After or during crossing of the plastid envelope, FAs are then re-esterified with coenzyme A (CoA) to form acyl-CoA, which is a major intermediate in seed oil biosynthesis. The FA moieties of acyl-CoA can be further elongated in the ER.

Both plastid-derived and elongated FA moieties make up the cytosolic acyl-CoAs, which are utilized as substrates by the membrane-bound acyltransferases of the sn-glycerol-3-phosphate (G3P) or Kennedy pathway of TAG biosynthesis (Stymne and Stobart, 1987, Pp 175-214, In: Stumpf, P. K. (ed.) The Biochemistry of Plants, Vol. 9, Lipids:Structure and Function. Academic Press, New York; Weselake, R. J., 2002, Pp 27-56, In: Kuo, T. M. and Gardner, H. W. (eds.) Lipid Biotechnology. Marcel Dekker, Inc., New York; Weselake, R. J., 2005, Pp 162-221, In: Murphy, D. J. (ed.) Plant Lipids-Biology, Utilization and Manipulation. Blackwell Publishing, Oxford). Acyl-CoA-independent reactions are also known to lead to TAG formation (Stobart et al., 1997, Planta 203:58-66; Dahlqvist et al., 2000, PNAS USA 97:6487-5492).

Phosphatidylcholine (PC) is an important intermediate in formation of polyunsaturated FAs (“PUFA”) by the membrane bound desaturases that act on the acyl group at the sn-2 position of PC (Jaworski, 1987, The Biochemistry of Plants 9:159-173). PUFAs formed on the PC molecule can be channelled back to the mainstream of TAG formation through the activity of phospholipid:diacylglycerol acyltransferase (PDAT), cholinephosphotransferase (CPT), phospholipase A₂ (PLA₂) or reverse reaction of lysophosphatidylcholine acyltransferase (LPCAT) (Weselake, R. J., 2005, Pp 162-221, In: Murphy, D. J. (ed.) Plant Lipids-Biology, Utilization and Manipulation. Blackwell Publishing, Oxford). The last two enzymes facilitate enrichment of the cytosolic acyl-CoA pool with PUFAs that can be used by acyltransferases of the Kennedy pathway.

Upon synthesis, TAG molecules accumulate within the lipid bilayer of the ER and pinch off as lipid droplets called oil bodies (OB) coated in a half-unit membrane composed of phospholipids (PL) and proteins (Huang, A. H. C., 1992, Ann. Rev. Plant Physiol and Plant Mol. Biol., 43:177-200; 1996, Plant Physiol 110:1055-1061). The major protein of the OB coat is oleosin, which has been shown to exhibit two isoforms in most higher plants (Qu and Huang, 1990, J. Biol. Chem. 265:2238-2243). Oleosins play crucial roles in seed maturation and germination, protecting OB from the action of cytosolic phospholipases and from coalescence during seed desiccation, and acting as possible docking sites for lipases during re-mobilization of the seed lipid storage (Tzen and Huang, 1992, Journal of Cell Biology 117:327-335; Beisson et al., 2001, Biochimica et Biophysica Acta 1531:47-58). Oleosins are synthesized on the surface of the ER before being targeted to OB, and are specifically enriched in the ER regions involved in TAG formation and OB biogenesis (Hills et al., 1993, Planta 189:24-29; Lacey et al., 1999, The Plant Journal 17:397-405). Specificity of oleosin targeting to OB has been used as a basis for developing a commercial technology for expression of oleosin-target protein fusions to produce value-added proteins in plants (van Rooijen and Moloney, 1995, Bio/Technology 13:72-77; Nykiforuk et al. 2006, Plant Biotechnology Journal 4:77-85).

The attempts to alter FA composition of seed oil through genetic engineering have been based mostly on manipulating the genes encoding enzymes of FA biosynthesis (Gunstone and Pollard, 2001, Pp 155-184, In: Gunstone, F. D. (ed.) Structured and Modified Lipids. Marcel Dekker, New York; Thelen and Ohlrogge, 2002, Metabolic Engineering 4:12-21). Reduction of undesirable FA content and increase in valuable FA formation can be achieved by up- or down-regulation of catalytic activities of specific steps in the FA biosynthetic pathway. If the goal of a seed oil modification program is to introduce a novel or unusual FA into the seed oil, engineering of the entire biosynthetic pathway may be required. However, successful modification of the cytosolic acyl-CoA pool composition does not always result in desirable changes in the FA composition of TAG. One of the reasons for discrimination of different acyl-CoA species for incorporation into TAG is the substrate selectivity of acyltransferases that can limit channelling of particular FAs from the acyl-CoA pool into seed oil (Katavic et al., 2000, Biochemical Society Transactions 28:935-937). This problem can be overcome to a certain extent by modification of selectivity/specificity properties of the native acyltransferases though molecular engineering (e.g., site-directed mutagenesis, DNA shuffling), or by introduction of foreign acyltransferases with desirable properties. Another problem researchers encounter when trying to engineer novel FA biosynthetic pathways in plants is an inefficient channelling of the acyl-groups between the substrate forms (acyl-CoA- and PC-esterified acyl chain) utilized in different catalytic steps of the pathway (Abbadi et al., 2004, The Plant Cell 16:2734-2748). Thus, modification of enzyme activities may need to be complemented by manipulation of systems responsible for the trafficking of FA moieties between cellular locations and between different substrate pools.

Acyl-CoA binding proteins (ACBPs) are small housekeeping proteins ubiquitously found in all eukaryotic organisms studied to date (Færgeman and Knudsen, 2002, Biochem. J. 368:679-682; Burton et al., 2005, Biochem. J. 392:299-307). These proteins specifically bind long-chain acyl-CoAs with high affinity with 1:1 molar ratio (Rasmussen et al., 1990, Biochem. J. 265:849-855). Although, the physiological role of acyl CoA binding proteins in cellular metabolism is not clear, a number of functions have been assigned to these lipid binding proteins including maintenance and protection of the cytosolic acyl-CoA pool from hydrolysis, intracellular transport of acyl-CoA, and protection of the cellular membranes from detergent activity of acyl-CoAs (Engeseth et al., 1996, Archives of Biochemistry and Biophysics 331:55-62; Mandrup et al., 1993, Biochem. J. 290:369-374; Cohen Simonsen et al., 2003, FEBS Letters 552:253-258). Acyl CoA binding proteins have also been proposed to have a role in the regulation of enzyme activities and gene expression (Mogensen et al., 1987, Biochem. J. 241:189-192; Rassmussen et al., 1993, Biochem. J. 292:907-913; 1994, Biochem. J. 299:165-170; Petrescu et al., 2003, The Journal of Biological Chemistry 278:51813-51824). Overexpression of acyl CoA binding protein in yeast and in animal systems has been shown to increase the acyl-CoA pool size (due to an increase in certain acyl-CoA species) and rates of glycerolipid synthesis (Mandrup et al., 1993, Biochem. J. 290:369-374; Huang et al., 2005, Biochemistry 44:10282-10297). Studies in A. thaliana revealed a six-membered acyl CoA binding protein gene family encoding proteins that differ in structure, cellular location and binding properties, suggesting different roles in lipid metabolism (Engeseth et al., 1996, Archives of Biochemistry and Biophysics 331:55-62; Chye et al., 2000, Plant Mol. Biol. 44:711-721; Leung et al., 2004, Plant Mol. Biol. 55:297-309). The only B. napus acyl CoA binding protein identified so far, which represents a small cytosolic protein of 92 amino acids, had elevated levels of expression in developing embryos and flowers compared to other parts of the plant (Hills et al., 1994, Plant Mol. Biol. 25:917-920). More careful examination of acyl CoA binding protein expression in developing seeds revealed that the highest concentration of the protein coincided with the peak of TAG accumulation (Engeseth et al., 1996, Archives of Biochemistry and Biophysics 331:55-62). Also, the results of in vitro experiments showed that recombinant B. napus acyl CoA binding protein (rACBP) stimulated glycerol-3-phosphate acyltransferase (GPAT) activity in a manner dependent on acyl CoA binding protein:acyl-CoA ratio in the reaction mixture (Brown et al., 1998, Plant Physiol. Biochem. 36:629-635). Taken together, these findings suggest that acyl CoA binding protein may have an important role in TAG accumulation in developing seeds. Studying the binding properties of recombinant B. napus acyl CoA binding protein showed that the protein had a higher affinity towards oleoyl-CoA (18:1-CoA) than palmitoyl-CoA (16:0-CoA), suggesting that binding/transport of some acyl-CoA species by the protein may be preferred over the others (Brown et al., 1998, Plant Pysiol. Biochem. 36:629-635).

Expression of acyl CoA binding protein in several heterologous hosts, including plants, has been disclosed previously (Bergmüller et al., 2001, Poster No. 12, German Society for Fat Science Working Group Plant Lipids Symposium. Plant Lipid Metabolism: From Basic Research to Biotechnology, July 2001. Meisdorf, Germany; Enikeev and Mishutina, 2005, Russian Journal of Plant Physiology 52:668-671). However Bergmüller et al. disclose that no change was observed in the levels or composition of levels of fatty acid present in transgenic plants. Enikeev and Mishutina, disclose that, depending on the genetic construct and the Brassica cultivar that is used, erucic acid levels may be modulated in Brassica. However, Enikeev was not concerned with changes in the overall levels of fatty acids, while the levels of polyunsaturated fatty acids remain unchanged.

In view of the shortcomings in the prior art, there is a need in the art to improve methods for the modulation of plant oils.

### SUMMARY OF THE INVENTION

The present disclosure generally relates to methods for the modulation of plant oils. In particular, the present disclosure relates to plants that have been genetically modified to increase the overall level of oil, or levels of polyunsaturated fatty acids in plants, or both. More in particular, the present disclosure relates to plants that have been genetically modified to express an acyl-CoA binding protein (ACBP) within the plant seeds to improve or enhance the levels of polyunsaturated fatty acids in these plants, or for increasing the level of oil in these plants, or both. Accordingly, the present disclosure provides a method for increasing the level of polyunsaturated fatty acids in plants, or for increasing the level of oil, or both, the method comprising the steps of:

(a) providing a chimeric nucleic acid construct comprising, in the 5′ to 3′ direction of transcription as operably linked components:
(i) a nucleic acid sequence capable of controlling expression in plant seed cells; and
(ii) a nucleic acid sequence encoding an acyl CoA binding protein;

(b) introducing the chimeric nucleic acid construct into a plant cell; and

(c) growing the plant cell into a mature plant capable of expressing the acyl-CoA binding protein within the plant seeds.

In accordance with the present disclosure, it has been found that plant seeds may be particularly advantageously used to increase the level of polyunsaturated fatty acids, or oil, or both in a plant through the use of a seed preferred promoter. In particular, as demonstrated in the Examples, expression of acyl-CoA binding proteins (ACBPs) under the control of a seed preferred promoter increased levels of polyunsaturated fatty acid (PUFA) in seed oil as compared to wild type controls. In contrast, expression of ACBPs under the control of a constitutive promoter showed a decrease in PUFA levels in seed oil. Accordingly, the present disclosure provides a method for increasing the levels of polyunsaturated fatty acids in plant seeds, or for increasing the level of oil, or both, the method comprising the steps of:

(a) providing a chimeric nucleic acid construct comprising in the 5′ to 3′ direction of transcription as operably linked components:
(i) a nucleic acid sequence capable of controlling expression in plant seed cells in a seed preferred manner; and
(ii) a nucleic acid sequence encoding an acyl-CoA binding protein;

(b) introducing the chimeric nucleic acid construct into a plant cell; and

(c) growing the plant cell into a mature plant capable of setting seed wherein the seed expresses the acyl-CoA binding protein.

In a further preferred embodiment, the nucleic acid sequence capable of controlling expression in a plant seed cell is a seed preferred promoter comprising an abscicic acid response element (“ABRE”).

In a particularly preferred embodiment of the present disclosure the nucleic acid sequence capable of controlling expression in a plant seed cell is the phaseolin promoter.

In further preferred embodiments, the chimeric nucleic acid sequence further comprises a nucleic acid sequence encoding a targeting or stabilizing polypeptide linked in reading frame to the nucleic acid sequence encoding the acyl CoA binding protein. Preferably the targeting or stabilizing polypeptide is a polypeptide that, in the absence of the acyl CoA can readily be expressed and stably accumulates in a plant cell. The targeting or stabilizing protein may be plant specific or non-plant specific. Plant-specific targeting or stabilizing polypeptides that can be used in accordance with the present disclosure include an oilbody protein, such as an oleosin. Non-plant specific targeting or stabilizing polypeptides that may be used in accordance herewith include single chain antibodies, actin, tubulin, tubulin binding protein or trinectin. The plant-specific or non-plant specific targeting or stabilizing polypeptide may be linked to the acyl-CoA binding protein. In particularly preferred embodiments, the targeting or stabilizing protein is a protein capable of the directing the acyl-CoA binding protein to the plant oil bodies, to the cytoplasm or to the endoplasmic reticulum (ER).

Nucleic acid sequences that may be used in accordance herewith to stabilize or target the acyl CoA binding protein to the ER include for example nucleic acid sequences encoding KDEL, HDEL, SDEL sequences. Nucleic acid sequences that encode polypeptides that may be used to target the acyl CoA binding protein to an oil body include nucleic acid sequences encoding oil body proteins, such as oleosins, or fragments or variations thereof. In yet a further preferred embodiment, the nucleic acid sequence encoding the acyl CoA binding protein is expressed in such a manner that the acyl CoA binding protein accumulates in the cytoplasm. In such an embodiment, the nucleic acid sequence may not comprise a targeting signal.

In a further preferred embodiment, the chimeric nucleic acid construct is introduced into the plant cell under nuclear genomic integration conditions where the chimeric nucleic acid sequence is stably integrated in the plant's genome.

In a yet further preferred embodiment the nucleic acid sequence encoding acyl CoA binding protein is optimized for plant codon usage. Preferred nucleic acid sequences used in accordance with the present disclosure encode a Brassica napus acyl CoA binding protein (SEQ ID NO:2).

In another aspect, the present disclosure provides a method of obtaining plant seed comprising an increased level of polyunsaturated fatty acids, or for increasing the level of oil, or both. Accordingly, pursuant to the present disclosure a method is provided for obtaining plant seed comprising:

(a) providing a chimeric nucleic acid construct comprising in the 5′ to 3′ direction of transcription as operably linked components:
(i) a nucleic acid sequence capable of controlling expression in seed cells; and
(ii) a nucleic acid sequence encoding an acyl CoA binding protein;

(b) introducing the chimeric nucleic acid construct into a plant cell;

(c) growing the plant cell into a mature plant capable of setting seed; and

(d) obtaining seed from said plant wherein the seed comprises increased levels of polyunsaturated fatty acids, or increased level of oil, or both, relative to wild type plants.

Preferably the levels of polyunsaturated fatty acids in the plant seed oil is increased relative to the level of polyunsaturated fatty acids in plant seed oil of plants not comprising the chimeric nucleic acid construct of the present disclosure, by no less than 1%, more preferably no less than 2%, and more preferably no less than 3% and more preferably no less than 4%, and more preferably no less than 5%

Preferably, the overall levels of plant seed oil is increased relative to the level of oil in plants not comprising the chimeric nucleic acid construct of the present disclosure, by no less than 1% (absolute wt.), more preferably by no less than 2%, and more preferably by no less than 3% and more preferably no less than 4%, and more preferably by no less than 5%, and more preferably by no less than 6%, and more preferably by no less than 7%, and more preferably by no less than 8%, and more preferably by no less than 9%, and more preferably by no less than 10%.

The seeds may be used to obtain a population of progeny plants each comprising a plurality of seeds expressing acyl-CoA binding protein. The present disclosure also provides plants capable of setting seed having an increased level of polyunsaturated fatty acids. In a preferred embodiment of the present disclosure, the plants capable of setting seed comprise a chimeric nucleic acid sequence comprising in the 5′ to 3′ direction of transcription:

(a) a first nucleic acid sequence capable of controlling expression in a plant seed cell operatively linked to;

(b) a second nucleic acid sequence encoding an acyl-CoA binding protein polypeptide.

In a preferred embodiment the chimeric nucleic acid sequence is integrated in the plant's nuclear genome.

In a further preferred embodiment of the present disclosure the plant that is used is an Arabidopsis plant or a Carthamus plant, and in a particularly preferred embodiment, the plant is a Brassica plant.

In yet another aspect, the present disclosure provides plant seeds expressing acyl CoA binding protein. In a preferred embodiment of the present disclosure, the plant seeds comprise a chimeric nucleic acid sequence comprising in the 5′ to 3′ direction of transcription:

(a) a first nucleic acid sequence capable of controlling expression in a plant cell operatively linked to;

(b) a second nucleic acid sequence encoding an acyl CoA binding protein

The seeds are a source whence the desired oil enhanced in polyunsaturated fatty acids, which is synthesized by the seed cells, may be extracted and obtained in a more or less pure form. The polyunsaturated fatty acids may be used for nutritional, nutraceutical, pharmaceutical, industrial and other purposes.

Without being restricted to a theory, the applicants believe that directed expression of acyl CoA binding protein may trap specific acyl-CoA species for triacylglycerol biosynthesis.

Thus, the present disclosure relates to the incorporation of acyl-CoA binding sites as a means of trapping specific acyl-CoA species for incorporation into TAG biosynthesis. More particularly, it relates to the use of acyl CoA binding protein as molecular tool to modify fatty acid composition and seed oil content.

The present disclosure is intended to encompass B. napus acyl CoA binding protein (SEQ ID NO:2), and variants and fragments thereof.

Overexpression of acyl CoA binding protein in the cytosol may change the acyl CoA binding protein:acyl-CoA ratio and affect the rate of acyl-CoA exchange between the cytosolic pool and acyl-CoA producing/consuming systems. Thus, a modulation of the FA composition and content of seed oil by means of seed preferred expression of acyl CoA binding protein targeted to the oil body or overexpression in the cytosol may be achieved in accordance with the present disclosure.

Other features and advantages of the present disclosure will become apparent from the following detailed description. It should be understood, however, that the detailed description and the specific examples while indicating preferred embodiments of the present disclosure are given by way of illustration only, since various changes and modifications within the spirit and scope of the present disclosure will become apparent to those skilled in the art from this detailed description.

### BRIEF DESCRIPTION OF THE DRAWINGS

In the drawings, like elements are assigned like reference numerals. The drawings are not necessarily to scale, with the emphasis instead placed upon the principles of the present disclosure. Additionally, each of the embodiments depicted are but one of a number of possible arrangements utilizing the fundamental concepts of the present disclosure.

The drawings are briefly described as follows:

FIG. 1 is a schematic view of channeling of acyl-CoAs in pathways of seed oil formation.

FIG. 2 is a flowchart showing the experimental methods used in the transformation of A. thaliana and determination of results. Shaded rectangles connected with block arrows represent the sequence of performed procedures. Rectangles with no fill represent the method(s) used in the corresponding procedure.

FIG. 3 shows a schematic of the genetic constructs used in the transformation of A. thaliana.

FIG. 4 shows a vector map for pSBS 4140 (Oleosin-ACBP-1 under the seed preferred control of the phaseolin promoter) (SEQ ID NO:96).

FIG. 5 shows a vector map for pSBS4141 (ACBP-1-Oleosin fusion under the seed preferred control of the phaseolin promoter) (SEQ ID NO:97).

FIG. 6 shows a vector map for pSBS4142 (B82-Oleosin-ACBP-1 under the seed preferred control of the phaseolin promoter) (SEQ ID NO:98).

FIG. 7 shows a vector map for pSBS4143 (OleosinH3P-ACBP-1 under the seed preferred control of the phaseolin promoter) (SEQ ID NO:99).

FIG. 8 shows a vector map for pSBS4144 (PRS-ACBP-1 with KDEL retention signal under the seed preferred control of the phaseolin promoter) (SEQ ID NO:100).

FIG. 9 shows a vector map for pSBS4145 (PRS-D9-ACBP-1 with KDEL retention signal under the seed preferred control of the phaseolin promoter) (SEQ ID NO:101).

FIG. 10 shows a vector map for pSBS4146 (cytosolic ACBP-1 under the seed preferred control of the phaseolin promoter) (SEQ ID NO:102).

FIG. 11 shows a vector map pSBS4147 (cytosolic D9-ACBP-1 fusion under the seed preferred control of the phaseolin promoter) (SEQ ID NO:103).

FIG. 12 shows a vector map for pSBS4152 (ACBP-1 under the control of the 35S promoter) (SEQ ID NO:104).

FIG. 13 shows a vector map for pSBS4153 (ACBP—oleosin fusion under the control of the 35S promoter) (SEQ ID NO:105).

FIG. 14 shows a vector map for pSBS4154 (ACBP-KDEL under the control of the 35S promoter) (SEQ ID NO:106).

FIG. 15 shows the Western Blot analysis of transgene products (infrared fluorescence detection at 800 nm) using antibody directed against ACBP (A) or oleosin (B) as described in Example 7. Total seed protein of recombinant seeds were compared to a negative control (WT). (C) correlation between ACBP and PUFA content in developing T3 seeds expressing constructs 2, 4, 5, 6, 7, and 8 (as depicted in FIG. 3) at 16 days after flowering (DAF). For (A) and (B) lanes are as follows Mark=molecular weight marker (Precision Plus Protein™ Standards, Bio-Rad) (autofluoresence red at 700 nm), 1=ACBP-oleosin fusion protein (construct 2), 2=oleosinH3P-ACBP fusion protein (construct 4), 3=ACBP-KDEL retention signal (construct 5), 4=D9-ACBP-KDEL fusion protein (construct 6), 5=ACBP (construct 7), 6=D9-ACBP fusion protein (construct 8), WT=wild type TSP extract.

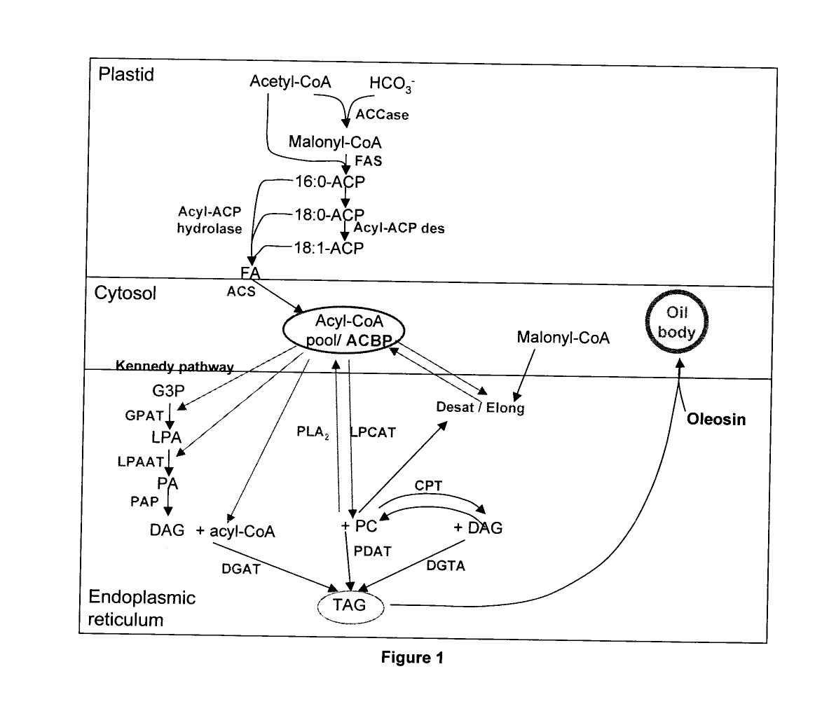

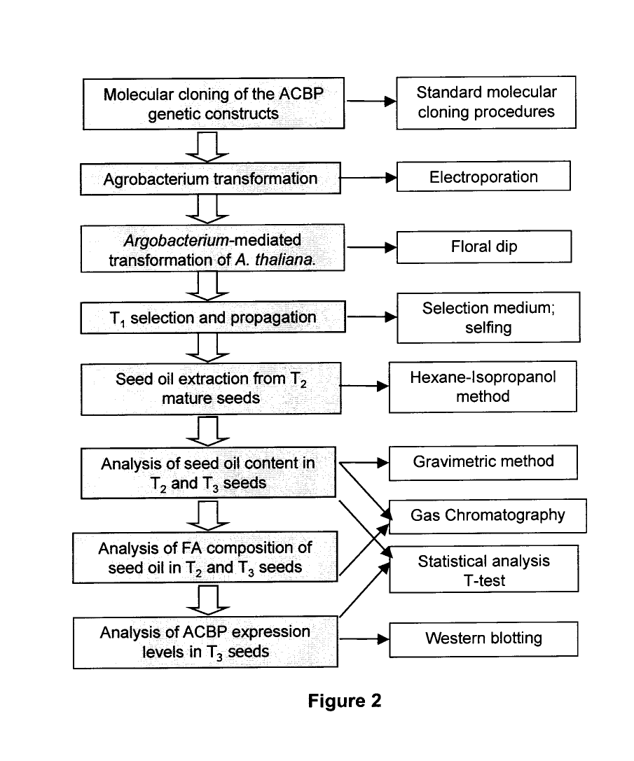

### DETAILED DESCRIPTION OF PREFERRED EMBODIMENTS

### Terms and Definitions

In the present disclosure, all terms not defined herein have their common art-recognized meanings. Where permitted, all patents, applications, published applications, and other publications, including nucleic acid and polypeptide sequences from GenBank, SwissProt and other databases referred to in the disclosure are incorporated by reference in their entirety. To the extent that the following description is of a specific embodiment or a particular use of the disclosure, it is intended to be illustrative only, and not limiting of the claimed disclosure. The following description is intended to cover all alternatives, modifications and equivalents that are included in the spirit and scope of the disclosure, as defined in the appended claims.

As used herein, the terms “acyl CoA binding protein”; “acyl CoA binding polypeptide” and “ACBP” refer to any and all polypeptide sequences of an acyl CoA binding protein including, without limitation, those listed in Table 2 and preferably SEQ ID NOs: 1 to 33. Acyl CoA binding proteins or polypeptides further include any and all polypeptides comprising a sequence of amino acid residues which is (i) substantially identical to the amino acid sequences constituting any acyl CoA binding protein polypeptides set forth herein or (ii) encoded by a nucleic acid sequence capable of hybridizing under at least moderately stringent conditions to any nucleic acid sequence encoding acyl CoA binding protein set forth herein or capable of hybridizing under at least moderately stringent conditions to any nucleic acid sequence encoding acyl CoA binding protein set forth herein but for the use of synonymous codons.

By the phrase “at least moderately stringent hybridization conditions”, it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tₘ, which in sodium containing buffers is a function of the sodium ion concentration and temperature (Tₘ=81.5° C.−16.6 (Log₁₀[Na])+0.41(% (G+C)−600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tₘ, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at T. (based on the above equation)−5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “chimeric” as used herein in the context of nucleic acid sequences refers to at least two linked nucleic acid sequences which are not naturally linked. Chimeric nucleic acid sequences include linked nucleic acid sequences of different natural origins. For example a nucleic acid sequence constituting a plant promoter linked to a nucleic acid sequence encoding an acyl CoA binding protein is considered chimeric. Chimeric nucleic acid sequences also may comprise nucleic acid sequences of the same natural origin, provided they are not naturally linked. For example a nucleic acid sequence constituting a promoter obtained from a particular cell-type may be linked to a nucleic acid sequence encoding a polypeptide obtained from that same cell-type, but not normally linked to the nucleic acid sequence constituting the promoter. Chimeric nucleic acid sequences also include nucleic acid sequences comprising any naturally occurring nucleic acid sequence linked to any non-naturally occurring nucleic acid sequence.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present disclosure may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The terms “nucleic acid sequence encoding an acyl CoA binding protein” and “nucleic acid sequence encoding an acyl CoA binding protein polypeptide”, which may be used interchangeably herein, refer to any and all nucleic acid sequences encoding an acyl CoA binding protein polypeptide including, without limitation, those sequences identified in Table 1, preferably SEQ ID NOs:1 to 33. Nucleic acid sequences encoding an acyl CoA binding protein polypeptide further include any and all nucleic acid sequences which (i) encode polypeptides that are substantially identical to the acyl CoA binding protein polypeptide sequences set forth herein; or (ii) hybridize to any acyl CoA binding protein nucleic acid sequences set forth herein under at least moderately stringent hybridization conditions or which would hybridize thereto under at least moderately stringent conditions but for the use of synonymous codons.

The term “nucleic acid sequence capable of controlling expression in plant seed cells” refers to any and all nucleic acid sequences that cause expression of the acyl CoA binding protein in plant seeds.

The term “nucleic acid sequence capable of controlling expression in plant seeds cells in a seed-preferred manner” or “seed-preferred promoter” includes any and all nucleic acid sequences that cause expression of the acyl CoA binding protein predominantly in the seeds of the plant with little or no expression in other tissues. Preferably, “seed preferred promoters” (or “seed specific promoters”) are promoters which control expression of the acyl CoA binding protein so that preferably at least 80% of the total amount of ACBP present in the mature plant is present in the seed. More preferably, at least 90% of the total amount of ACBP protein present in the mature plant is present in the seed. Most preferably, at least 95% of the total amount of recombinant protein present in the mature plant is present in the seed.

By the term “substantially identical” it is meant that two polypeptide sequences preferably are at least 70% identical, and more preferably are at least 85% identical and most preferably at least 95% identical, for example 96%, 97%, 98% or 99% identical. In order to determine the percentage of identity between two polypeptide sequences the amino acid sequences of such two sequences are aligned, using for example the alignment method of Needleman and Wunsch (1970, J. Mol. Biol. 48: 443), as revised by Smith and Waterman (1981, Adv. Appl. Math. 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (1988, SIAM J. Applied Math. 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., 1984, Nucleic Acids Res. 12: 387) BLASTP, BLASTN and FASTA (Altschul et al., 1990, J. Molec. Biol. 215: 403). A particularly preferred method for determining the percentage identity between two polypeptides involves the Clustal W algorithm (Thompson, J D, Higgines, D G and Gibson T J, 1994, Nucleic Acid Res 22(22): 4673-4680) together with the BLOSUM 62 scoring matrix (Henikoff S & Henikoff, J G, 1992, Proc. Natl. Acad. Sci. USA 89: 10915-10919) using a gap opening penalty of 10 and a gap extension penalty of 0.1, so that the highest order match obtained between two sequences wherein at least 50% of the total length of one of the two sequences is involved in the alignment.

The term “increasing levels of polyunsaturated fatty acids” or “increased levels of polyunsaturated fatty acids” or “increase in PUFA” as used herein means that, relative to a control, the level of at least one PUFA is increased in the seed oil, more preferably the combined level of more than one PUFA is increased in the seed oil and most preferably, the combined levels of linolenic (18:2) fatty acid and linoleic (18:3) fatty acid are increased in the seed oil. Control as used in herewith is a plant not transformed with the chimeric nucleic acid sequence of the present disclosure (i.e. wildtype plant).

### Preparation of Recombinant Expression Vectors Comprising Chimeric Nucleic Acid Sequences Encoding Acyl CoA Binding Protein and a Nucleic Acid Sequence Capable of Controlling Expression in a Plant Cell

When a Brassica napus ACBP was heterologously over-expressed as chimeric nucleic acid constructs operably linked in the 5′ to 3′ direction in different configurations (seed-specific versus constitutive manner, as chimeric fusions versus non-fusions and different cellular compartments) significant increases in the polyunsaturated fatty acid (PUFA) content of mature seeds was observed. In general, the increase in PUFA was at the expense of long chain (C20) monounsaturated fatty acids (LC-MUFA) and the effect was heritable. Biochemical analysis of seed oil from transgenic lines of two plant generations (T₂ and T₃) revealed significant increase in linolenic (18:2) fatty acid (up to 33.77±1.51 vs. 27.08±0.15% weight in WT) and decrease in 20:1 (to 14.71±1.45 vs. 19.99±0.76% weight in WT). Also, most of the transgenic lines showed a decrease in stearidonic (18:0) and linoleic (18:3) fatty acids in seed oil. Overall, transgenic plants expressing ACBP from the seed preferred promoter (5 out of 8 constructs) had an increased amount of PUFA in seed oil comparing to a wild type control (52.58±0.49 vs. 48.34±0.23% weight in WT), at the expense of monounsaturated fatty acids (MUFA) (down to 32.65±1.16 vs. 38.29±0.69% weight in WT). Contrarily, transgenic plants expressing ACBP under the control of a constitutive promoter showed a decrease in PUFA and increase in MUFA content in seed oil. Protein analysis showed that transgenic ACBP expressed from the seed preferred promoter was present in developing and mature seeds at detectable levels.

Accordingly, the present disclosure generally relates to methods for the modulation of plant oils. In particular, the disclosure relates to plants that have been genetically modified to increase the levels of polyunsaturated fatty acids and levels of oils in plant seeds. More particularly, the present disclosure relates to plants that have been genetically modified to express an acyl-CoA binding protein within plant seeds to improve or enhance the levels of polyunsaturated fatty acids in these plant seeds. Accordingly, the present disclosure provides a method for increasing the level of polyunsaturated fatty acids, or plant oils, or both, in plant cells comprising:

(a) providing a chimeric nucleic acid construct comprising in the 5′ to 3′ direction of transcription as operably linked components:
(i) a nucleic acid sequence capable of controlling expression in plant cells; and
(ii) a nucleic acid sequence encoding an acyl CoA binding protein;

(b) introducing the chimeric nucleic acid construct into a plant cell; and

(c) growing the plant cell into a mature plant wherein the acyl-CoA binding protein is expressed in the plant.

In accordance with the present disclosure, it has been found that plant seeds may be used to increase the level of polyunsaturated fatty acids, or plant oils, or both, in a plant through the use of a seed preferred promoter. Expressing the ACBP under the control of a seed-preferred promoter results in increased levels of PUFA in the seed oil than PUFA levels in the seed oil of wild type plants or in the seed oil of plants that express ACBP using a constitutive promoter. Accordingly, the present disclosure comprises a method for increasing the levels of polyunsaturated fatty acids and/or oil in plant seeds comprising the steps of:

(a) providing a chimeric nucleic acid construct comprising in the 5′ to 3′ direction of transcription as operably linked components:
(i) a nucleic acid sequence capable of controlling expression in plant seed cells in a seed-preferred manner; and
(ii) a nucleic acid sequence encoding an acyl-CoA binding protein;

(b) introducing the chimeric nucleic acid construct into a plant cell; and

(c) growing the plant cell into a mature plant capable of setting seed wherein the acyl-CoA binding protein is expressed in seed and results in an increased level of polyunsaturated fatty acids or oil in the plant seeds.

The nucleic acid sequences encoding an acyl CoA binding protein that may be used in accordance with the methods and compositions provided herein may be any nucleic acid sequence encoding an acyl CoA binding protein polypeptide.

Preferred nucleic acid sequences encoding acyl CoA binding proteins sequences that may be used include any nucleic acid sequences encoding an acyl CoA binding protein and preferably the polypeptide chains set forth in Table 1, preferably SEQ ID NOs: 1 to 33. The respective corresponding nucleic acid sequences encoding the acyl CoA binding protein polypeptides can be readily identified via the Accession identifier numbers provided in

Table 1. Using these nucleic acid sequences, additional novel acyl CoA binding protein encoding nucleic acid sequences may be readily identified using techniques known to those of skill in the art. For example libraries, such as expression libraries, cDNA and genomic libraries, may be screened, and databases containing sequence information from sequencing projects may be searched for similar sequences. Alternative methods to isolate additional nucleic acid sequences encoding acyl CoA binding protein polypeptides may be used, and novel sequences may be discovered and used in accordance with the present disclosure. In preferred embodiments, nucleic acid sequences encoding acyl CoA binding proteins are plant, algae or fish acyl CoA binding proteins, including SEQ ID NOs: 1 to 33. In more preferred embodiments, nucleic acid sequences encoding acyl CoA binding proteins are plant acyl CoA binding proteins.

Acyl CoA binding protein homologues have been identified in all four eukaryotic kingdoms, Animalia, Plantae, Fungi and Protista, and eleven eubacterial species. To date acyl CoA binding protein homologues have not been detected in any other known bacterial species, or in archaea. Many bacterial, fungal and higher eukaryotic species only harbour a single acyl CoA binding protein homologue. However, a number of species, ranging from protozoa to vertebrates, have evolved two to six lineage-specific paralogues through gene duplication and/or retrotransposition events. The acyl CoA binding protein is highly conserved across phylums (Burton et al., 2005, Biochem J., 392(Pt 2): 299-307). The present disclosure is intended to encompass all homologues, paralogues and analogs of acyl CoA binding protein, variants and fragments thereof, provided however that (i) such paralogues, analogs, variants and fragments are substantially identical to one of the acyl CoA binding proteins set forth herein and/or (ii) the nucleic acid sequence encoding such paralogues, analogs, variants and fragments are capable of hybridizing under at least moderately stringent hybridization conditions to a nucleic sequence encoding the acyl CoA binding proteins set forth herein. Analogs that may be used herein include acyl CoA binding protein molecules wherein a variety of natural and synthetic mutations and modifications have been discovered including, but not limited to, point mutations, deletion mutations, frameshift mutations and chemical modifications. Alterations to the nucleic acid sequence encoding acyl CoA binding protein to prepare acyl CoA binding protein analogs may be made using a variety of nucleic acid modification techniques known to those skilled in the art, including, for example site directed mutagenesis, targeted mutagenesis, random mutagenesis, the addition of organic solvents, gene shuffling or a combination of these and other techniques known to those of skill in the art (Shraishi et al., 1988, Arch. Biochem. Biophys, 358: 104-115; Galkin et al., 1997, Protein Eng. 10: 687-690; Carugo et al., 1997, Proteins 28: 10-28; Hurley et al., 1996, Biochem, 35:5670-5678; Holmberg et al., 1999, Protein Eng. 12:851-856).

In accordance herewith the nucleic acid sequence encoding acyl CoA binding protein is linked to a nucleic acid sequence capable of controlling expression of the acyl CoA binding protein polypeptide in a plant seed cell. Accordingly, the present disclosure also comprises a nucleic acid sequence encoding acyl CoA binding protein linked to a promoter capable of controlling expression in a plant seed cell. Nucleic acid sequences capable of controlling expression in plant cells that may be used herein include any plant derived promoter capable of controlling expression of polypeptides in plant seeds.

In a preferred embodiment, the nucleic acid sequence capable of controlling expression in a plant cell is a seed-preferred promoter. In such an embodiment, a promoter which results in preferential expression of the acyl CoA binding protein polypeptide in seed tissue is used.

The present inventors have found that in accordance herewith promoters selected from the group of promoters comprising an abscicic acid response element or ABRE are particularly preferred. As used herein “ABRE” is defined as a nucleic sequence located within 2000 base pairs upstream (5′) from the transcriptional start site of a nucleic acid sequence encoding a polypeptide and capable of conferring to that nucleic acid sequence responsiveness to abscisic acid (“ABA response”). As used herein “ABA response” is defined as an increase of at least two times the amount of transcript from a gene, when excised plant embryos, microspore derived embryos or cell suspension cultures are exposed to a concentration of 10 μM exogenously supplied abscisic acid compared to the amount of transcript from said gene when excised plant embryos, microspore derived embryos or cell suspension cultures are exposed to basal media lacking abscisic acid as further described in Delisle and Crouch, 1989; Plant Physiol. 91:617-623). Preferably, the ABRE comprises less than 10 nucleic acid residues, comprising the nucleic acid sequence ACGT or ACGTG or ACCTG. More preferably the ABRE comprises a nucleic acid sequence selected from the group of nucleic acid sequences consisting of: (1) ACGT, (2) (G/C/T)ACGT(G/T)GC, (3) (C/T)ACGTGGC, (4) TGACGTGGG, (5) AAACGTGTC, (6) ACACGTGGC, (7) ACACCTGAC) and (8) ACACNNG.

In a further preferred embodiment, the promoter comprises an ABRE and further comprises a promoter element selected from the group comprising: (1) RY Element; (2) E-box and (3) G-box. As used herein an RY Element is defined as a nucleic acid sequence located within 2000 bp from the transcriptional start site of a structural gene comprising the sequence (1) CATGCA or (2) CATGCA(C/T). The RY Elements are also known as the legumin box (Gatehouse et al., 1986; Philos. Trans. R. Soc. B314: 367-384) and Sph element (Kao et al., 1996, Plant Cell 8: 1171-1179. As used herein, the “E-box” is defined as nucleic acid sequence located within 2000 bp from the transcriptional start site of a structural gene comprising a basic region helix-loop-helix with the sequence CANNTG. As used herein, the “G-box” is defined as a nucleic acid sequence located within 2000 bp from the transcriptional start site of a structural gene comprising the sequence CACGTG.

Seed-preferred promoters that may be used in accordance with the present disclosure include, without limitation, the bean phaseolin promoter (SEQ ID NO:37) (Slightom, J. L., 1983, Proc. Natl. Acad. Sci. USA 80: 1897-1901); the Arabidopsis 18 kDa oleosin promoter (SEQ ID NO:36) (Van Rooijen, G. J. et al., 1992, Plant Mol Biol 18: 1177-1179; U.S. Pat. No. 5,792,922); the flax 16 kDa oleosin promoter (SEQ ID NO:34) (WO 01/16340); the flax 18 KDa oleosin promoter (SEQ ID NO:35) (WO 01/16340); the flax legumin like seed storage protein (linin) promoter (SEQ ID NO:41) (WO 01/16340); the Brassica napus napin promoter (SEQ ID NO:38) (Josefsson, L G., 1987, J Biol Chem 262: 12196-12201); the Brassica napus cruciferin promoter (SEQ ID NO:39) (GenBank M93103); the Brassica napus cruciferin promoter isolated by SemBioSys Genetics Inc. (SEQ ID NO:40) and the bean arcelin promoter (SEQ ID NO: 107) (Jaeger G D, et al., 2002, Nat. Biotechnol . . . Dec; 20:1265-8) and any promoter sequences capable of hybridizing to the aforementioned promoters under at least moderately stringent hybridization conditions. Table 2 provides a summary of some of the above seed-preferred promoters including the identification and location of various consensus sequences. New promoters useful in various plants are constantly discovered. Numerous examples of seed preferred promoters may be found in Ohamuro et al. (1989, Biochem. of Plants 15: 1-82), Thomas (1993, The Plant Cell 5:1401-1410), and Goossens et al. (1999, Plant Physiol. 120:1095-1104).

In preferred embodiments, the chimeric nucleic acid sequence further comprises a nucleic acid sequence encoding a stabilizing polypeptide linked in reading frame to the nucleic acid sequence encoding the acyl CoA binding protein. The stabilizing polypeptide is used to facilitate protein folding and/or enhance the stable accumulation of the acyl CoA binding protein in plant cells. In addition, or alternatively, the stabilizing polypeptide may be used to target the acyl CoA binding protein to a desired location within the plant cell, preferably the cytoplasm or cytosol. Preferably the stabilizing polypeptide is a polypeptide that in the absence of the acyl CoA binding protein can readily be expressed and stably accumulates in transgenic plant cells. The stabilizing polypeptide may be a plant specific or non-plant specific polypeptide. Plant-specific stabilizing polypeptides that can be used in accordance with the present disclosure include oil body proteins including, but not limited to, the oil body proteins listed in Table 3. In a preferred embodiment the oil body protein is an oleosin, caleosin, or a steroleosin including, without limitation, the ones provided in SEQ ID NO:46 to 83. Non-plant specific stabilizing polypeptides that may be used in accordance herewith single chain antibodies or fragments thereof. Preferably, non-plant specific stabilizing polypeptides are codon optimized for optimal expression in plants.

Single chain antibodies or antibodies that are preferably used herein include single chain antibodies or fragments thereof are capable of associating with an oil body protein obtainable from the seed in which the acyl CoA binding protein is expressed, i.e. in an embodiment of the present disclosure in which Arabidopsis plant cells are used, a single chain antibody or fragment thereof is selected which is capable of associating with an Arabidopsis oil body protein. In a further preferred embodiment, the single chain antibody is a single chain FV antibody capable of specifically associating with the 18 kDa oleosin from Arabidopsis thaliana (D9scFv). The term “single chain antibody fragment” (scFv) or “antibody fragment” as used herein means a polypeptide containing a variable light (V_{L}) domain linked to a variable heavy (V_{H}) domain by a peptide linker (L), represented by V_{L}-L-V_{H}. The order of the V_{L} and V_{H} domains can be reversed to obtain polypeptides represented as V_{H}-L-V_{L}. “Domain” is a segment of protein that assumes a discrete function, such as antigen binding or antigen recognition. The single chain antibody fragments for use in the present disclosure can be derived from the light and/or heavy chain variable domains of any antibody. Preferably, the light and heavy chain variable domains are specific for the same antigen. In one embodiment, the antigen is an oil body protein. In another embodiment, the antigen is associated with the endoplasmic reticulum. The individual antibody fragments which are joined to form a multivalent single chain antibody may be directed against the same antigen or can be directed against different antigens. Methodologies to create single chain antibodies are well known in the art. For example single chain antibodies can be created by screening single chain (scFV) phage display libraries.

Methodologies to create single chain antibodies from phage display libraries are well known in the art. McCafferty et al. (1990, Nature 348:552-554) demonstrated the use of a phage-display system in which fragments of antibodies were expressed as a fusion protein with a fd phage vector to allow for the expression of single chain antibodies on the surface of the phage. The production of a single chain antibody phage display library can be achieved using for example, the Recombinant Phage Antibody System developed by Amersham Biosciences and Cambridge Antibody Technology. A more detailed protocol is available from Amersham Biosciences which is sold in 3 parts including a mouse scFV molecule, an expression module and a detection module. Briefly, the protocol for the production of single chain antibodies is as follows. Messenger RNA can be obtained from either a mouse hybridoma or mouse spleen cells from a mouse that has been immunized with the antigen of interest. The mouse hybridoma represents the most abundant source for the antibody gene to be cloned, as it expresses the heavy and light chain genes for a single antibody but antibodies can also be cloned using spleen cells from an immunized mouse. The mRNA is converted to cDNA using a reverse transcriptase and random hexamer primers. The use of random hexamers will result in cDNA molecules that are sufficient in length to clone the variable regions of the heavy and light chain molecules. After the cDNA molecules are created, primary PCR reactions are performed to amplify the heavy and light variable regions separately. Primers are designed to amplify the heavy or light chain variable region by hybridizing to opposite ends of the chain. Once the variable regions are amplified, the PCR reactions are subjected to agarose gel electrophoresis and gel purified to remove the primers and any extraneous PCR products. Once the heavy and light chain variable regions have been purified they are assembled into a single gene using a linker. The linker region is designed to ensure that the correct reading frame is maintained between the heavy and light chain. For example, the variable heavy (V_{H}) and variable light (V_{L}) chains may be linked using a (Gly₄Ser)₃ linker to obtain a single chain antibody fragment (scFv) of approximately 750 base pairs in length. Once the heavy and light chains are assembled with the linker a secondary PCR reaction is performed to amplify the assembled scFV DNA fragments. Primers should be designed to introduce restriction sites to allow for cloning into phagemid expression vectors. For example Sfi I and Not I sites can be added to the 5′ and 3′ end of these scFv gene for cloning into the pCANTAB 5 E vector (Amersham Biosciences). Once PCR is complete, the DNA fragments should be purified to remove unincorporated primers and dNTPs. This can be achieved using spun-column purification. Once the DNA fragments have been purified and quantified the fragments are digested with the appropriate restriction enzymes to allow for cloning into the appropriate expression vector. The DNA fragments are subsequently ligated into an expression vector, for example pCANTAB 5E (Amersham Biosciences) and introduced into competent E. coli cells. The cells should be grown on appropriate selection media to ensure that only cells containing the expression vector will grow (i.e. using a specific carbon source and antibiotic selection). Once the E. coli is grown, the phagemid-containing colonies are infected with a M13 helper phage (i.e. KO7—Amersham Biosciences) to yield recombinant phage which display the scFv fragments. The M13 phage will initiate phage replication and complete phage particles will be produced and released from the cells, expressing scFv species on their surface. The phage displaying the correct scFv antibodies are identified by panning using the specific antigen. To eliminate the non-specific phage, the culture of recombinant phage can be transferred to an antigen-coated support (i.e. a flask or a tube), and washed. Only those phage displaying the correct scFv will be bound to the support. A susceptible strain of E. coli is subsequently infected with the phage bound to the antigen-coated support. The phage can be enriched by rescuing with the helper phage and panning against the antigen multiple times or can be plated directly onto a solid medium without further enrichment. The E. coli cells that have been infected with the phage selected against the appropriate antigen are plated and individual colonies are picked. Phage, from the individual colonies, are then assayed using for example the ELISA assay (enzyme-linked immunosorbent assay). Phage antibodies which are positive using the ELISA assay can then be used to infect E. coli HB2151 cells for the production of soluble recombinant antibodies. Once the appropriate clones are selected the sequence of the scFv antibody gene can be identified and used for the present disclosure.

In specific embodiments, the chimeric nucleic acid sequence further comprises a targeting polypeptide. A “targeting polypeptide” as used herein means any amino acid sequence capable of directing the acyl CoA binding protein polypeptide, when expressed, to a desired location within the plant cell. The present inventors have found that particularly suitable targeting signals that may be used herein, are those capable of targeting the acyl CoA binding protein polypeptide to an oil body, the cytosol, the cytoplasm or the ER.

In order to achieve accumulation of the acyl CoA binding protein in the ER or an oil body, the acyl CoA binding protein is linked to a targeting polypeptide which causes the acyl CoA binding protein to be retained in the ER or an oil body. In one embodiment, the targeting signal that is capable of retaining the acyl CoA binding protein in the ER contains a C-terminal ER-retention motif. Examples of such C-terminal ER-retention motifs include KDEL, HDEL, DDEL, ADEL and SDEL sequences. Other examples include HDEF (Lehmann et al., 2001, Plant Physiol. 127(2): 436-439), or two arginine residues close to the N-terminus located at positions 2 and 3, 3 and 4, or 4 and 5 (Abstract from Plant Biology 2001 Program, ASPB, July 2001, Providence, R.I., USA). Nucleic acid sequences encoding a C-terminal retention motif are preferably linked to the nucleic acid sequence encoding the acyl CoA binding protein in such a manner that the polypeptide capable of retaining the acyl CoA binding protein in the ER is linked to the C-terminal end of the acyl CoA binding protein polypeptide. In one embodiment, the C-terminal ER retention motif is KDEL.

In embodiments in which the acyl CoA binding protein is retained in the ER, the chimeric nucleic acid sequence additionally may comprise a nucleic acid sequence which encodes a polypeptide which targets the acyl CoA binding protein to the endomembrane system (“signal peptide”). In embodiments in which the acyl CoA binding protein polypeptide is retained in the ER using a sequence, such as KDEL, HDEL or SDEL polypeptide, it is particularly desirable to include a nucleic acid sequence encoding a signal peptide. Exemplary signal peptides that may be used herein include the tobacco pathogenesis related protein (PRS) signal sequence (Sijmons et al., 1990, Bio/technology, 8:217-221), lectin signal sequence (Boehn et al., 2000, Transgenic Res, 9(6):477-86), signal sequence from the hydroxyproline-rich glycoprotein from Phaseolus vulgaris (Yan et al., 1997, Plant Phyiol. 115(3):915-24; Corbin et al., 1987, Mol Cell Biol 7(12):4337-44), potato patatin signal sequence (Iturriaga, G et al., 1989, Plant Cell 1:381-390; Bevan et al., 1986, Nuc. Acids Res. 41:4625-4638.) and the barley alpha amylase signal sequence (Rasmussen and Johansson, 1992, Plant Mol. Biol. 18(2):423-7).

In a preferred embodiment, the acyl CoA binding protein polypeptide is linked to a polypeptide that is capable of directing the acyl CoA binding protein polypeptide to an oil body. In a preferred embodiment, the acyl CoA binding protein is linked to an oil body protein. Oil body proteins that may be used in this regard include any protein that naturally associates with an oil body, including those oil body proteins identified in Table 1, preferably SEQ ID NOs:46 to 83. The respective corresponding nucleic acid sequences encoding the oil body protein polypeptide chains can be readily identified via the Accession identifier numbers provided in Table 3. In addition, modified oleosins may also be used including the ones described in WO 2004/113376. Using these nucleic acid sequences, additional novel oil body proteins encoding nucleic acid sequences may be readily identified using techniques known to those of skill in the art. For example libraries, such as expression libraries, cDNA and genomic libraries, may be screened, and databases containing sequence information from sequencing projects may be searched for similar sequences. Alternative methods to isolate additional nucleic acid sequences encoding oil body protein polypeptides may be used, and novel sequences may be discovered and used in accordance with the present disclosure. Oil body proteins that are particularly preferred are oleosins, for example a corn oleosin (including SEQ ID NO:63 to 70) (Bowman-Vance et al., 1987, J. Biol. Chem. 262: 11275-11279; Qu et al., 1990, J. Biol. Chem. 265:2238-2243) or Brassica oleosin (including SEQ ID NO:51 to 60) (Lee et al., 1991, Plant Physiol. 96:1395-1397), caleosins (including SEQ ID NO:71 to 78), see for example Genbank accession number AF067857) and steroleosins (Lin et al., 2002 Plant Physiol. 128(4):1200-11). In a further preferred embodiment, the oil body protein is a plant oleosin and shares sequence similarity with other plant oleosins such as the oleosin isolated from Arabidopsis thaliana (SEQ ID NO: 79) or Brassica napus (SEQ ID NO:80). In another embodiment, the oil body protein is a caleosin or calcium binding protein from plant, fungal or other sources and shares sequence homology with plant caleosins such as the caleosin isolated from Arabidopsis thaliana (SEQ ID NO:81 and SEQ ID NO:82). In another embodiment the oil body protein is a steroleosin (SEQ ID NO:83), or a sterol binding dehydrogenase (Lin L-J et al, 2002, Plant Physiol 128:1200-1211). In a preferred embodiment., the oil body protein may be a modified oil body protein. It has been shown that oil body targeting of oleosin is disrupted by alteration of its membrane topology caused by structural modifications in the hydrophobic domain (Abell et al., 2004, J. Biol. Chem. 277:8602-8610). Therefore, in one embodiment, modified oleosin genes were used to disrupt the oleosin-acyl CoA binding protein targeting to oil bodies. One modified oleosin gene product with a short hydrophobic domain is expected to have a more stable membrane topology in the ER and to be more labile within the membrane compared to native oleosin (OleoH3P, Siloto, R. M. P. 2005. Analysis of structure-function of plant seed oleosins. PhD Dissertation. University of Calgary, Alberta). In another embodiment, oleosin may be modified by the addition of the N′-terminal signal peptide (luminal “anchor”), which inhibits oleosin transition from ER to oil bodies.

Polypeptides capable of retaining the acyl CoA binding protein in the ER or an oil body are typically not cleaved and the acyl CoA binding protein may accumulate in the form of a fusion protein, which is, for example, typically the case when a KDEL retention signal is used to retain the polypeptide in the ER or when an oil body protein is used to retain the polypeptide in an oil body.

In a further preferred embodiment, the nucleic acid sequence encoding the acyl CoA binding protein is expressed in such a manner that the acyl CoA binding protein accumulates in the cytoplasm. In such an embodiment the nucleic acid sequence may not comprise a targeting signal. In such an embodiment, the acyl CoA binding protein may be linked to a stabilizing polypeptide, such as a single chain antibody (Arabidopsis thaliana D9scFv). Alternatively, in such an embodiment, the chimeric nucleic acid sequence may comprise a nucleic acid sequence encoding a targeting or stabilizing polypeptide operatively linked in-frame to the nucleic acid sequence encoding the acyl CoA binding protein. In these instances the linked polypeptide may increase the stability and/or expression levels of acyl CoA binding protein by “scaffolding” to itself (dimerization, trimerization, oligomerization) or associating with the existing infrastructurally related proteins (including organellar surfaces) within the cell. The targeting or stabilizing polypeptides that may be used in accordance herewith include examples such as actin, tubulin, tubulin binding protein or trinectin.

The chimeric nucleic acid sequence may also comprise a nucleotide sequence encoding N- and/or C-terminal polypeptide extensions. Such extensions may be used to stabilize and/or assist in folding of the acyl CoA binding protein poly peptide chain or they may facilitate targeting to a compartment in the cell, for example the oil body. Polypeptide extensions that may be used in this regard may be implemented by, for example, a nucleic acid sequence encoding a single chain antibody or combinations of such polypeptides. Single chain antibody extensions that are particularly desirable include those that permit association of the acyl CoA binding protein with an oil body. Such extensions are preferably included in embodiments in which the acyl CoA binding protein is expressed in the plant seed and targeted within the seed cell to the ER.

Certain genetic elements capable of enhancing expression of the acyl CoA binding protein polypeptide may be used herein. These elements include the untranslated leader sequences from certain viruses, such as the AMV leader sequence (Jobling and Gehrke, 1987, Nature, 325: 622-625) and the intron associated with the maize ubiquitin promoter (U.S. Pat. No. 5,504,200). Generally the chimeric nucleic acid sequence will be prepared so that genetic elements capable of enhancing expression will be located 5′ to the nucleic acid sequence encoding the acyl CoA binding protein polypeptide.

The present disclosure further includes the chimeric nucleic acid constructs described above. Accordingly, in one embodiment, the disclosure provides a chimeric nucleic acid construct comprising in the 5′ to 3′ direction of transcription:

(a) a first nucleic acid sequence capable of controlling expression in a plant cell in a seed-preferred manner operatively linked to;

(b) a second nucleic acid sequence encoding an acyl-CoA binding protein polypeptide.

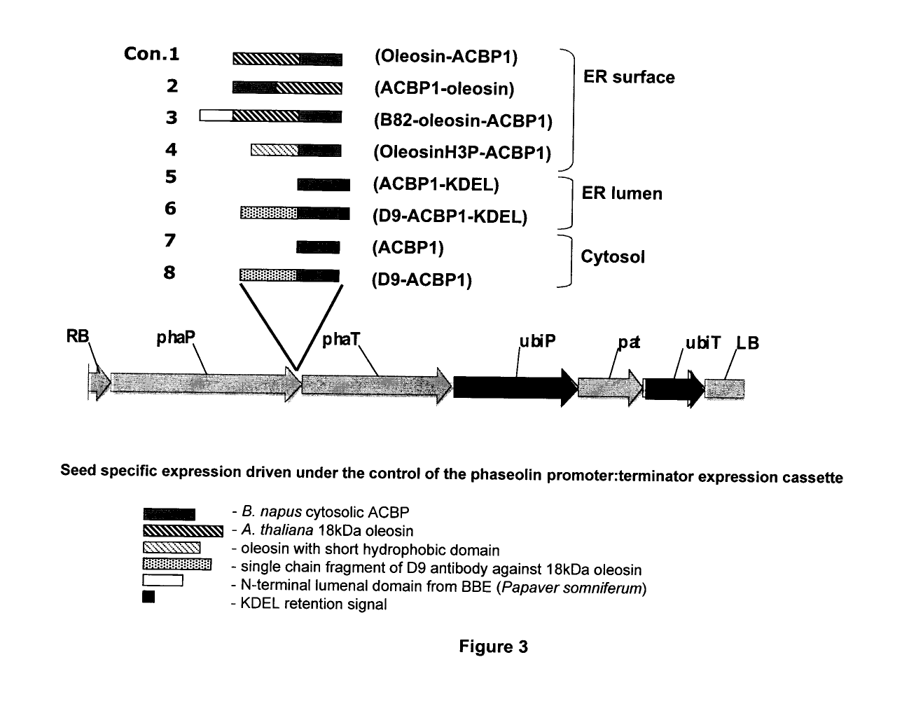

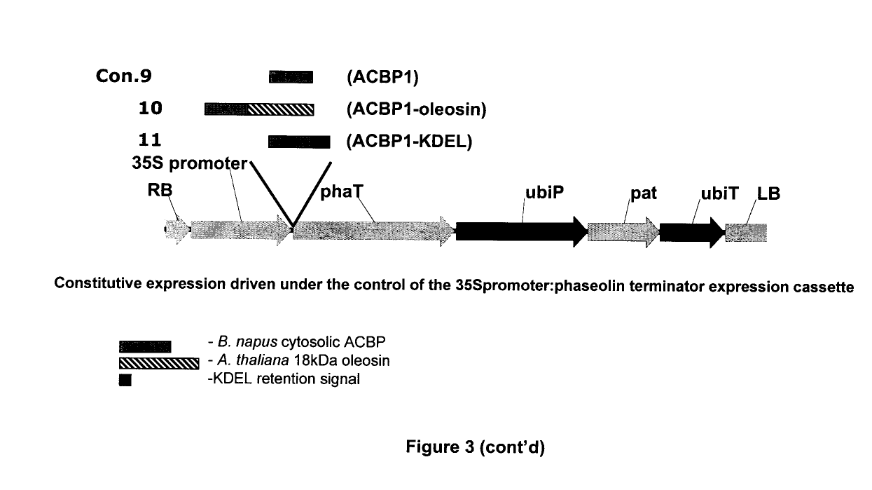

As mentioned previously, the nucleic acid sequence capable of controlling expression in plant seed is preferably a seed preferred promoter comprising an ABRE element. Specific nucleic acid constructs that have been prepared are shown in FIG. 3 and SEQ ID NOS:96-106. Preferably the construct has a sequence shown in SEQ ID NOS:96-103. In a specific embodiment, the construct has the sequence shown in SEQ ID NO:102 which comprises ACBP under the control of a phaseolin promoter. In another embodiment, the construct has the sequence of SEQ ID NO:103 which comprises ACBP under the control of the phaseolin promoter and also comprise a single chain antibody that binds to an oil body protein.

In accordance with the present disclosure the chimeric nucleic acid sequences comprising a promoter capable of controlling expression in plant linked to a nucleic acid sequence encoding an acyl CoA binding protein polypeptide can be integrated into a recombinant expression vector which ensures good expression in the cell. Accordingly, the present disclosure includes recombinant expression vectors comprising the chimeric nucleic acid sequences of the present disclosure, wherein the expression vector is suitable for expression in a plant cell. The term “suitable for expression in a plant cell” means that the recombinant expression vector comprises the chimeric nucleic acid sequence of the present disclosure linked to genetic elements required to achieve expression in a plant cell. Genetic elements that may be included in the expression vector in this regard include a transcriptional termination region, one or more nucleic acid sequences encoding marker genes, one or more origins of replication and the like. In preferred embodiments, the expression vector further comprises genetic elements required for the integration of the vector or a portion thereof in the plant cell's nuclear genome, for example the T-DNA left and right border sequences which facilitate the integration into the plant's nuclear genome in embodiments of the disclosure in which plant cells are transformed using Agrobacterium. In a further preferred embodiment said plant cell is a plant seed cell.

As mentioned above, the recombinant expression vector generally comprises a transcriptional terminator which besides serving as a signal for transcription termination further may serve as a protective element capable of extending the mRNA half life (Guarneros et al., 1982, Proc. Natl. Acad. Sci. USA, 79: 238-242). The transcriptional terminator is generally from about 200 nucleotides to about 1000 nucleotides and the expression vector is prepared so that the transcriptional terminator is located 3′ of the nucleic acid sequence encoding acyl CoA binding protein. Termination sequences that may be used herein include, for example, the nopaline termination region (Bevan et al., 1983, Nucl. Acids. Res., 11: 369-385), the phaseolin terminator (van der Geest et al., 1994, Plant J. 6: 413-423), the arcelin terminator (Jaeger G D, et al., 2002, Nat. Biotechnol. 20:1265-8), the terminator for the octopine synthase genes of Agrobacterium tumefaciens or other similarly functioning elements. Transcriptional terminators may be obtained as described by An (1987, Methods in Enzym. 153: 292).

In one embodiment, the expression vector may further comprise a marker gene. Marker genes that may be used include all genes that allow the distinction of transformed cells from non-transformed cells, including all selectable and screenable marker genes. A marker gene may be a resistance marker such as an antibiotic resistance marker against, for example, kanamycin (U.S. Pat. No. 6,174,724), ampicillin, G418, bleomycin, hygromycin or spectinomycin which allows selection of a trait by chemical means or a tolerance marker against a chemical agent, such as the normally phytotoxic sugar mannose (Negrotto et al., 2000, Plant Cell Rep. 19: 798-803). Other convenient markers that may be used herein include markers capable of conveying resistance against herbicides such as glyphosate (U.S. Pat. Nos. 4,940,935; 5,188,642), phosphinothricin (U.S. Pat. No. 5,879,903) or sulphonyl ureas (U.S. Pat. No. 5,633,437). Resistance markers, when linked in close proximity to nucleic acid sequence encoding the acyl CoA binding protein polypeptide polypeptide, may be used to maintain selection pressure on a population of plant cells or plants that have not lost the nucleic acid sequence encoding the acyl CoA binding protein polypeptide. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

Recombinant vectors suitable for the introduction of nucleic acid sequences into plants include Agrobacterium and Rhizobium based vectors, such as the Ti and Ri plasmids, including for example pBIN19 (Bevan, 1984, Nucl. Acid. Res., 1984, 22: 8711-8721), pGKB5 (Bouchez et al., 1993, C R Acad. Sci. Paris, Life Sciences, 316:1188-1193), the pCGN series of binary vectors (McBride and Summerfelt, 1990, Plant Mol. Biol., 14:269-276) and other binary vectors (e.g. U.S. Pat. No. 4,940,838).

The recombinant expression vectors of the present disclosure may be prepared in accordance with methodologies well known to those skilled in the art of molecular biology. Such preparation will typically involve the bacterial species Escherichia coli as an intermediary cloning host. The preparation of the E. coli vectors as well as the plant transformation vectors may be accomplished using commonly known techniques such as restriction digestion, ligation, gel electrophoresis, DNA sequencing, the Polymerase Chain Reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Typically, these cloning vectors contain a marker allowing selection of transformed cells. Nucleic acid sequences may be introduced in these vectors, and the vectors may be introduced in E. coli grown in an appropriate medium. Recombinant expression vectors may readily be recovered from cells upon harvesting and lysing of the cells. Further, general guidance with respect to the preparation of recombinant vectors may be found in, for example: Sambrook et al., 1989, Molecular Cloning, a Laboratory Manual, Cold Spring Harbor Laboratory Press.

### Preparation of Plants Comprising Seed Capable of Expressing Acyl CoA Binding Protein

In accordance with the present disclosure, the chimeric nucleic acid sequence is introduced into a plant cell and the cells are grown into mature plants, wherein the plant expresses the acyl CoA binding protein polypeptide.

Any plant species or plant cell may be selected, preferably a plant capable of setting seed. Particular plants which may be used herein include cells obtainable from Arabidopsis thaliana, borage or starflower (Borago officinalis); Brazil nut (Betholettia excelsa); castor bean (Riccinus communis); coconut (Cocus nucifera); coriander (Coriandrum sativum); corn (Zea mays); cotton (Gossypium spp.); evening primrose (Oenothera spp); groundnut (Arachis hypogaea); jojoba (Simmondsia chinensis); linseed/flax (Linum usitatissimum); maize (Zea mays); mustard (Brassica spp. and Sinapis alba); oil palm (Elaeis guineensis); olive (Olea europaea); rapeseed (Brassica spp.); rice (Oryza sativa); safflower (Carthamus tinctorius); soybean (Glycine max); squash (Cucurbita maxima); barley (Hordeum vulgare); wheat (Triticum aestivum); duckweed (Lemnaceae sp), false flax (Camelina sp.) and sunflower (Helianthus annuus).

In accordance herewith in a preferred embodiment plant species or plant cells from oil seed plants are used. Oil seed plants that may be used herein include peanut (Arachis hypogaea); mustard (Brassica spp. and Sinapis alba); rapeseed (Brassica spp.); chickpea (Cicer arietinum); soybean (Glycine max); cotton (Gossypium hirsutum); sunflower (Helianthus annuus); lentil (Lens culinaris); linseed/flax (Linum usitatissimum); white clover (Trifolium repens); olive (Olea eurpaea); oil palm (Elaeis guineensis); safflower (Carthamus tinctorius); false flax (Camelina sp.); borage or starflower (Borago officinalis); evening primrose (Oenothera spp); and narbon bean (Vicia narbonesis).

In a particularly preferred embodiment Arabidopsis, carthamus, or Brassica spp. is used.

Methodologies to introduce plant recombinant expression vectors into a plant cell, also referred to herein as “transformation”, are well known to the art and typically vary depending on the plant cell that is selected. General techniques to introduce recombinant expression vectors in cells include, electroporation; chemically mediated techniques, for example CaCl₂ mediated nucleic acid uptake; particle bombardment (biolistics); the use of naturally infective nucleic acid sequences, for example virally derived nucleic acid sequences, or Agrobacterium or Rhizobium derived sequences, polyethylene glycol (PEG) mediated nucleic acid uptake, microinjection and the use of silicone carbide whiskers.

In preferred embodiments, a transformation methodology is selected which will allow the integration of the chimeric nucleic acid sequence in the plant cell's genome, and preferably the plant cell's nuclear genome. The use of such a methodology is preferred as it will result in the transfer of the chimeric nucleic acid sequence to progeny plants upon sexual reproduction. Transformation methods that may be used in this regard include biolistics and Agrobacterium mediated methods.

Transformation methodologies for dicotyledenous plant species are well known. Generally, Agrobacterium mediated transformation is used because of its high efficiency, as well as the general susceptibility by many, if not all, dicotyledenous plant species. Agrobacterium transformation generally involves the transfer of a binary vector, such as one of the hereinbefore mentioned binary vectors, comprising the chimeric nucleic acid sequence of the present disclosure from E. coli to a suitable Agrobacterium strain (e.g. EHA101 and LBA4404) by, for example, tri-parental mating with an E. coli strain carrying the recombinant binary vector and an E. coli strain carrying a helper plasmid capable of mobilizing the binary vector to the target Agrobacterium strain, or by DNA transformation of the Agrobacterium strain (Hofgen et al., Nucl. Acids. Res., 1988, 16:9877). Other techniques that may be used to transform dicotyledenous plant cells include biolistics (Sanford, 1988, Trends in Biotechn. 6:299-302); electroporation (Fromm et al., 1985, Proc. Natl. Acad. Sci. USA., 82:5824-5828); PEG mediated DNA uptake (Potrykus et al., 1985, Mol. Gen. Genetics, 199:169-177); microinjection (Reich et al., 1986, Bio/Techn. 4:1001-1004); and silicone carbide whiskers (Kaeppler et al., 1990, Plant Cell Rep., 9:415-418) or in planta transformation using, for example, a flower dipping methodology (Clough and Bent, 1998, Plant J., 16:735-743).

Monocotyledonous plant species may be transformed using a variety of methodologies including particle bombardment (Christou et al., 1991, Biotechn. 9:957-962; Weeks et al., 1993, Plant Physiol. 102:1077-1084; Gordon-Kamm et al., 1990, Plant Cell. 2:5603-618); PEG mediated DNA uptake (European Patents 0292 435; 0392 225) or Agrobacterium mediated transformation (Goto-Fumiyuki et al., 1999, Nature-Biotech. 17:282-286).

The exact plant transformation methodology may vary somewhat depending on the plant species and the plant cell type (e.g. seedling derived cell types such as hypocotyls and cotyledons or embryonic tissue) that is selected as the cell target for transformation. As mentioned above, in a particularly preferred embodiment, Brassica napus is used. A methodology to obtain safflower transformants is available in Baker and Dyer (Plant Cell Rep., 1996, 16:106-110). Additional plant species specific transformation protocols may be found in: Biotechnology in Agriculture and Forestry 46: Transgenic Crops I (1999, Y.P.S. Bajaj (ed.), Springer-Verlag, New York), and Biotechnology in Agriculture and Forestry 47: Transgenic Crops II (2001, Y.P.S. Bajaj (ed.), Springer-Verlag, New York.

Following transformation, the plant cells are grown and upon the emergence of differentiating tissue, such as shoots and roots, mature plants are regenerated. Typically a plurality of plants is regenerated. Methodologies to regenerate plants are generally plant species and cell type dependent and will be known to those skilled in the art. Further guidance with respect to plant tissue culture may be found in, for example: Plant. Cell and Tissue Culture, 1994, Vasil and Thorpe Eds., Kluwer Academic Publishers; and in: Plant Cell Culture Protocols (Methods in Molecular Biology 111), 1999, Hall Eds, Humana Press.

In one aspect, the present disclosure provides a method of obtaining plant seed comprising an increased level of polyunsaturated fatty acids or an increased overall level of oil, or both an increased level of polyunsaturated fatty acids and an increased overall level of oil. Accordingly, pursuant to the present disclosure a method is provided for obtaining plant seed comprising introducing chimeric nucleic acid constructs described herein into a plant cell, growing the plant cell into a mature plant; and obtaining seed from said plant wherein the seed comprises increased levels of polyunsaturated fatty acids relative to a control or increased overall levels of oil, or both. A control used in accordance herewith is a plant not transformed with the chimeric nucleic acid sequence of the present disclosure (i.e. wildtype plant). Preferably, the levels of polyunsaturated fatty acids in the plant seed oil is increased relative to the level of polyunsaturated fatty acids in plants not comprising the chimeric nucleic acid construct of the present disclosure, by no less than 1% (absolute wt.), more preferably by no less than 2%, and more preferably by no less than 3% and more preferably no less than 4%, and more preferably by no less than 5%, and more preferably by no less than 6%, and more preferably by no less than 7%, and more preferably by no less than 8%, and more preferably by no less than 9%.

Preferably, the overall levels of plant seed oil is increased relative to the level of oil in plants not comprising the chimeric nucleic acid construct of the present disclosure, by no less than 1% (absolute wt.), more preferably by no less than 2%, and more preferably by no less than 3% and more preferably no less than 4%, and more preferably by no less than 5%, and more preferably by no less than 6%, and more preferably by no less than 7%, and more preferably by no less than 8%, and more preferably by no less than 9%, and more preferably by no less than 10%, and more preferably by no less than 11%, and more preferably by no less than 12%, and more preferably by no less than 13%, and more preferably by no less than 14%.

It is noted that the term “no less than” also means “at least” and both can be used interchangeably herein.

The seeds may be used to obtain a population of progeny plants each comprising a plurality of seeds expressing acyl CoA binding protein. In preferred embodiments, a plurality of transformed plants is obtained, grown, and screened for the presence of the desired chimeric nucleic acid sequence, the presence of which in putative transformants may be tested by, for example, growth on a selective medium, where herbicide resistance markers are used, by direct application of the herbicide to the plant, or by Southern blotting. If the presence of the chimeric nucleic acid sequence is detected, transformed plants may be selected to generate progeny and ultimately mature plants comprising a plurality of seeds comprising the desired chimeric nucleic acid sequence. Such seeds may be used to isolate the plant seed oil or they may be planted to generate two or more subsequent generations. It will generally be desirable to plant a plurality of transgenic seeds to obtain a population of transgenic plants, each comprising seeds comprising a chimeric nucleic acid sequence encoding acyl CoA binding protein. Furthermore, it will generally be desirable to ensure homozygosity in the plants to ensure continued inheritance of the recombinant polypeptide. Methods for selecting homozygous plants are well known to those skilled in the art. Methods for obtaining homozygous plants that may be used include the preparation and transformation of haploid cells or tissues followed by the regeneration of haploid plantlets and subsequent conversion to diploid plants for example by the treatment with colchine or other microtubule disrupting agents. Plants may be grown in accordance with otherwise conventional agricultural practices.

Extraction of Seed Oil from Plants and Fatty Acid Analysis

In order to determine the fatty acid compositions in seeds, standard protocols for lipid extraction from mature seeds or developing embryos may be used, such as a hexane-isopropanol method (Siloto et al., 2006, The Plant Cell 18: 1961-1974) or a method as described by Bligh et al. (1959, Can. J. Biochem. Physiol. 37: 911-917). For example, seeds may be homogenized in liquid nitrogen and incubated at 70° C. for 10 min with 5 mL of isopropanol. The isopropanol may be evaporated under nitrogen, and lipids extracted with three extractions of chloroform, methanol, and water biphasic solutions (methanol:CHCl3:H₂O). The lipid fractions may be collected and the solvents completely evaporated under a nitrogen environment. Total lipids may be quantified by gravimetry after drying the samples in a desiccator for 24 h.

The subsequent analysis of fatty acid composition on isolated total lipids (acylated lipids and free fatty acids) can be performed by preparing non-reactive derivatives of fatty acids (FAMES; fatty acid methyl esters). In this procedure acylated lipids are transformed by a transmethylation reaction by which the glycerol moiety is displaced by another alcohol (methanol) in acidic conditions (HCl) (Siloto et al., 2006, The Plant Cell 18, 1961-1974). The preparation of methyl esters from isolated lipids and free fatty acids can also be done in alkaline conditions (Ichihara et al., 1996, Lipids 31, 535-539). Alternatively methyl esters can be obtained directly from a one-step procedure (Eras J et al., 2004, J Chromatogr A 1047: 157) or by combining lipid extraction and transesterification in situ on mature seeds or developing embryos with methanolic-HCL in the presence of toluene. FAMES are separated, identified and quantified by gas-liquid chromatography with flame ionization detection (GLC-FID)

### Partitioning of Seed Proteins to Assess Subcellular Targeting of Transgene Products.

Partitioning of seed proteins may be performed to determine if transgene products are correctly targeted to the oil bodies as expected for oleosin fused to ACBP and/or to determine if scFv D9 fusions are correctly folded (should associate with oil bodies during extraction). Conversely, if the transgene product is misfolded or aggregated in vivo, the recombinant protein may partition with the insoluble seed pellet following extraction. For this analysis proteins from mature seed or developing embryos may be partitioned into oil body (OB), buffer solubilized protein in the undernatant (UND) and insoluble proteins retained in the seed or embryo pellet (P). Samples derived from developing embryos (excised embryos from siliques selected DAF coincident with high triacylglycerol accumulation) or mature Arabidopsis seeds (25 mg) may be ground in 0.5 mL of extraction buffer (0.4 M sucrose, 0.5 M NaCl, and 50 mM Tris-HCl, pH 8.0). Samples may then be centrifuged at 10 000×g for 10 minutes to isolate oilbodies (OB) from buffer soluble (UND) and insoluble (P) seed proteins. Following centrifugation, the fat pad containing the OBs and UND may be decanted to a fresh microfuge tube. The remaining pellet (P) may be suspended in extraction buffer equal to a total volume of 1 mL and solubilized with the addition of 0.2 mL 10% SDS and boiled for 10 minutes. The OB and UND may subsequently be re-centrifuged at 10 000×g for 10 min to float the fat pad containing the OBs. The UND may be removed using a 26 G 5/8 1 ml syringe and transferred to a fresh clean tube. To the UND fraction, extraction buffer may be added to result in a total volume of 1 mL and solubilized with the addition of 0.2 mL 10% SDS followed by boiling for 10 minutes. The remaining OB fraction may be suspended in extraction buffer equal to a total volume of 1 mL and also solubilized with the addition of 0.2 mL 10% SDS followed by boiling for 10 minutes. Proteins associated with the OB, UND or P fraction may then be analyzed by SDS-PAGE using standard protocols (Sambrook et al., 1989, Molecular Cloning, a Laboratory Manual, Cold Spring Harbor Laboratory Press) and stained with Coomassie Brilliant Blue R 250 or blotted for Western analysis.

In another aspect, the present disclosure also provides plants capable of setting seed expressing acyl CoA binding protein. In a preferred embodiment of the disclosure, the plants capable of setting seed comprise a chimeric nucleic acid sequence comprising in the 5′ to 3′ direction of transcription:

(a) a first nucleic acid sequence capable of controlling expression in a plant seed cell operatively linked to;

(b) a second nucleic acid sequence encoding an acyl CoA binding protein polypeptide, wherein the seed contains acyl CoA binding protein.

Preferably the sequence capable of controlling expression in a plant seed cell is a seed preferred promoter comprising an ABRE.

In a preferred embodiment the chimeric nucleic acid sequence is stably integrated in the plant's nuclear genome.

In yet another aspect, the present disclosure provides plant seeds expressing acyl CoA binding protein. In a preferred embodiment of the present disclosure, the plant seeds comprise a chimeric nucleic acid sequence described herein.

The acyl CoA binding protein polypeptide may be present in a variety of different types of seed cells including, for example, the hypocotyls and the embryonic axis, including in the embryonic roots and embryonic leafs, and where monocotyledonous plant species, including cereals and corn, are used in the endosperm tissue.

The seeds may be used as a source of oil enhanced in polyunsaturated fatty acids, which is synthesized by the seed cells, and which may be extracted and obtained in a more or less pure form. The polyunsaturated fatty acids may be used for nutritional, nutraceutical, pharmaceutical, industrial and other purposes.

### EXAMPLES

The following examples are intended to exemplify embodiments of the disclosure, and not to limit the claimed disclosure in any manner.

### Example 1

### Molecular Cloning of Genetic Constructs for Acyl-CoA Binding Protein (ACBP) Expression

The standard molecular cloning procedures employed for preparation of the genetic constructs comprising acyl CoA binding protein are described herein in general terms. One such method is the Inoue method (Inoue H. et al., 1990, Gene 96:23-28) that reproducibly generates competent cultures of E. coli that yield 1×10⁸ to 3×10⁸ transformed colonies/mg of plasmid DNA.

DNA encoding an acyl-CoA binding site was synthesized by Picoscript based on the sequence of B. napus cytosolic acyl CoA binding protein cDNA available from the GenBank database (Accession number X77134) (SEQ ID NO: 108).

Eleven different constructs were created, shown schematically in FIG. 3 under the control of the annotated promoter:terminator cassettes for seed preferred versus constitutive expression:

1. B. napus acyl CoA binding protein cDNA (encoding for ACBP-1) fused in frame with the A. thaliana 18 kDa oleosin gene at the N′-terminus under the seed preferred control of a phaseolin promoter:terminator (SEQ ID NO: 96).
2. B. napus acyl CoA binding protein cDNA (encoding for ACBP-1) fused in frame with the A. thaliana 18 kDa oleosin gene at the C′-terminus under the seed preferred control of a phaseolin promoter:terminator (SEQ ID NO:97).
3. B. napus acyl CoA binding protein cDNA (encoding for ACBP-1) fused in frame with the A. thaliana 18 kDa oleosin gene modified with the addition of the luminal domain from Papaver somniferum berberine bridge enzyme (BBE) at the N′-terminus under the seed preferred control of a phaseolin promoter:terminator (SEQ ID NO:98)
4. B. napus acyl CoA binding protein cDNA (encoding for ACBP-1) fused in frame with the oleosin H3 Pgene at the N′-terminus under the seed preferred control of a phaseolin promoter:terminator (SEQ ID NO:99).
5. B. napus acyl CoA binding protein cDNA (encoding for ACBP-1) fused in frame with KDEL at the C′-terminus and an ER targeting signal peptide (PRS) fused in frame at the N′-terminus under the seed preferred control of a phaseolin promoter:terminator (SEQ ID NO:100).
6. B. napus acyl CoA binding protein cDNA (encoding for ACBP-1) fused in frame with and ER targeting signal peptide (PRS) and D9 at the N′-terminus and KDEL at the C′-terminus under the seed preferred control of a phaseolin promoter:terminator (SEQ ID NO:101).
7. B. napus acyl CoA binding protein cDNA (encoding for ACBP-1) by itself under the seed preferred control of a phaseolin promoter:terminator (SEQ ID NO:102).
8. B. napus acyl CoA binding protein cDNA (encoding for ACBP-1) fused in frame with D9 at the N′-terminus under the seed preferred control of a phaseolin promoter:terminator (SEQ ID NO:103).
9. B. napus acyl CoA binding protein cDNA (encoding for ACBP-1) under the constitutive control of a 35S promoter:phaseolin terminator (SEQ ID NO:104).
10. B. napus acyl CoA binding protein cDNA (encoding for ACBP-1) fusion in frame with A. thaliana 18 kDa oleosin gene at the C′-terminus under the constitutive control of a 35S promoter:phaseolin terminator (SEQ ID NO:105).
11. B. napus acyl CoA binding protein cDNA (encoding for ACBP-1) and KDEL under the constitutive control of a 35S promoter:phaseolin terminator (SEQ ID NO:106).

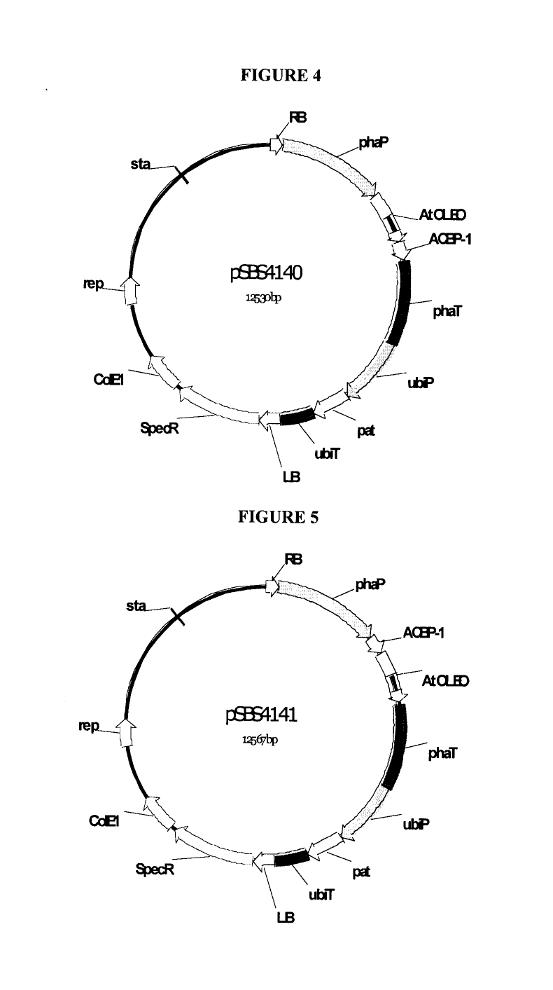

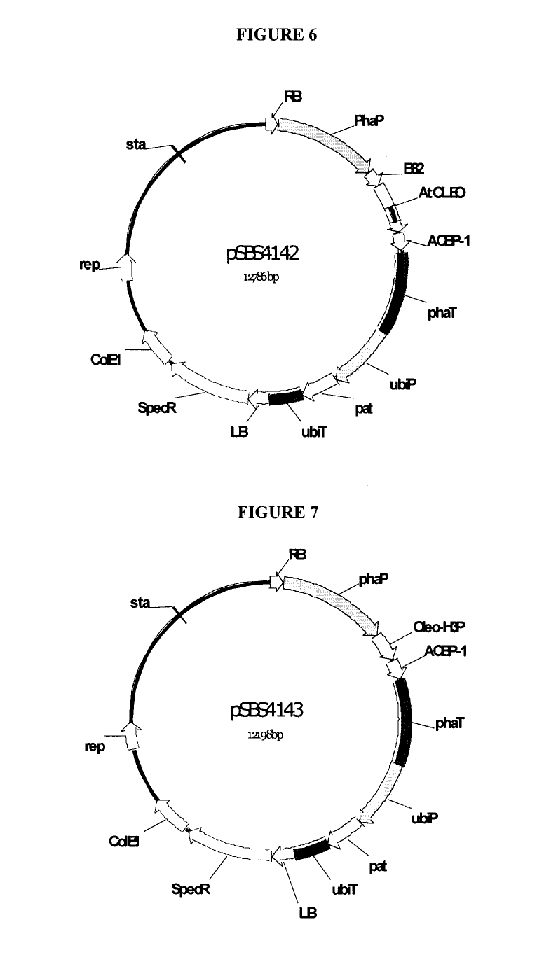

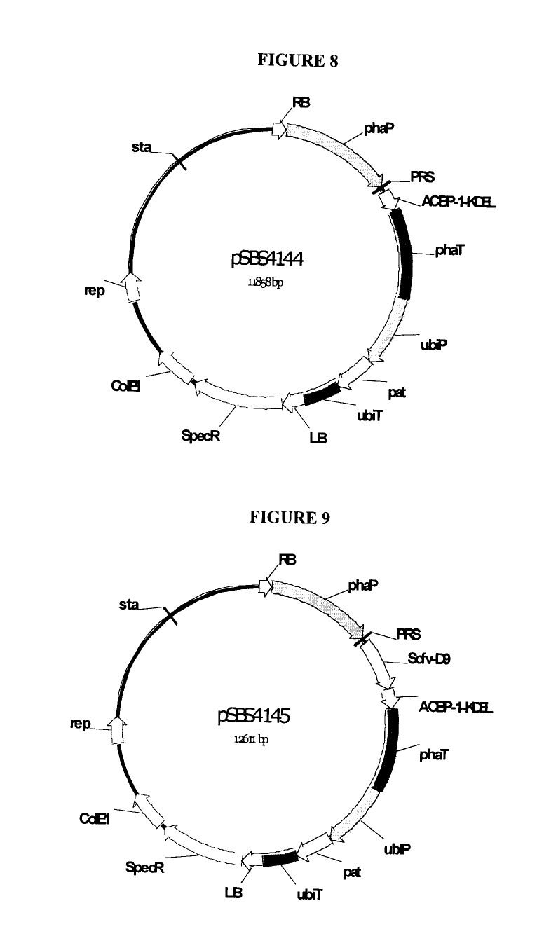

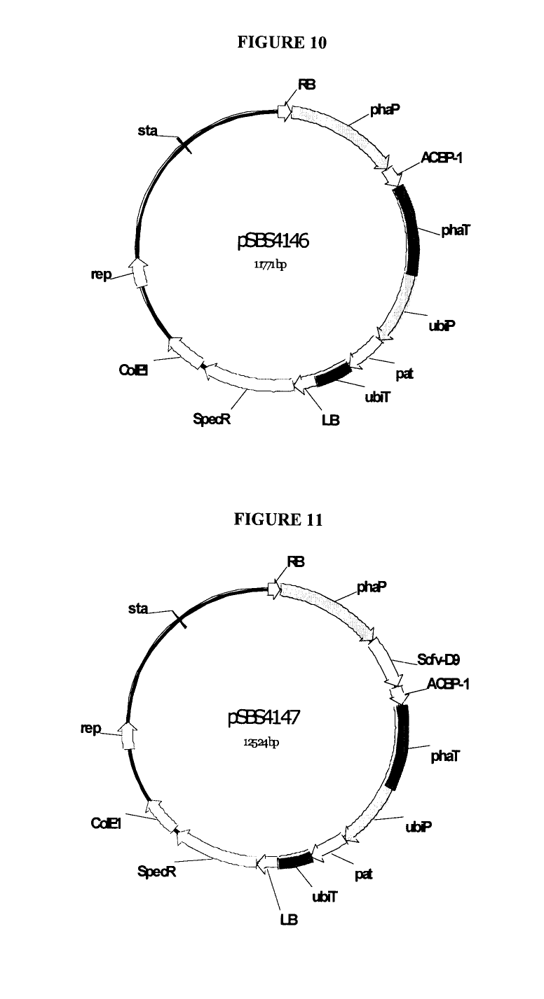

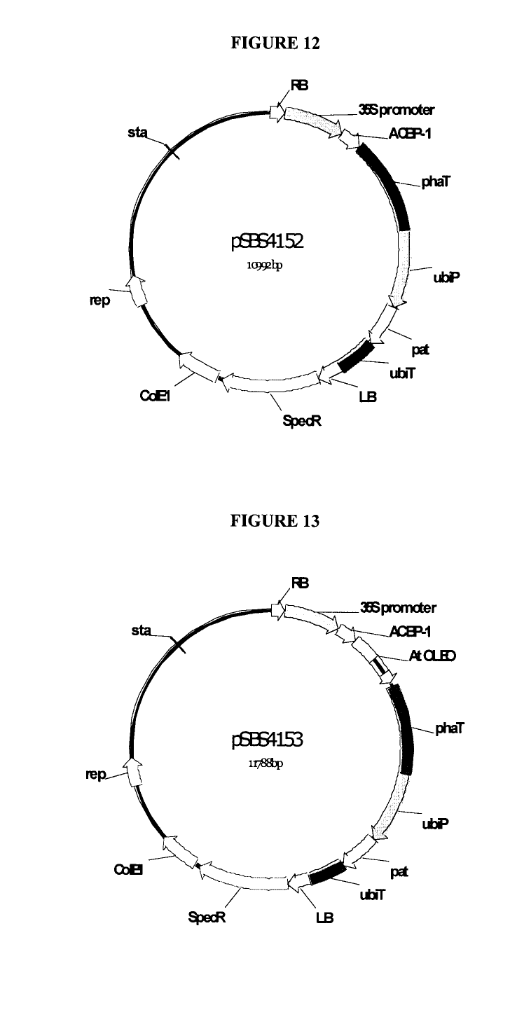

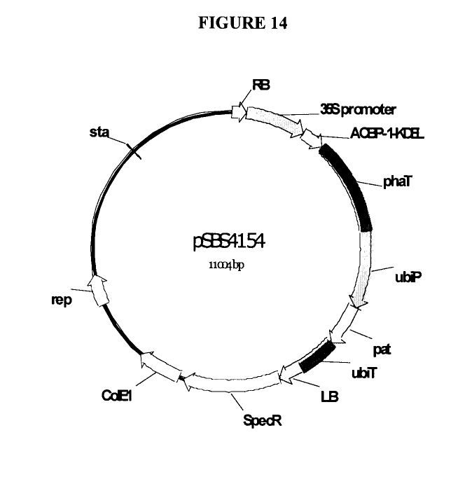

The vector maps shown in FIGS. 4-14 schematically map the genetic constructs referred to above, where ACBP-1—cytosolic acyl-CoA binding protein cDNA from Brassica napus (SEQ ID NO: 108) (Genbank X77134, Hills et al. 1994), ACBP-1 with KDEL—cytosolic acyl-CoA binding protein cDNA from Brassica napus with additional sequence for KDEL-retention signal. Oleosin—18 kDa oleosin gene from Arabidopsis thaliana. D9—single chain fragment of D9 antibody raised against 18 kDa oleosin.

### Example 2

### Agrobacterium Transformation

Standard protocols are available for the transformation of Agrobacterium (such as CSH Protocols; 2006, doi:10.1101/pdb.prot4665, which was adapted from “How to Transform Arabidopsis,” Chapter 5, in Arabidopsis by Detlef Weigel and Jane Glazebrook. Cold Spring Harbor Laboratory Press, Cold Spring Harbor, N.Y., USA, 2002).

### Preparation of Competent Agrobacterium Cells

In brief, competent cells were prepared by inoculation of 500 ml of LB (not YEP) with 5 ml of a fresh saturated culture of Agrobacterium tumefaciens. The culture was incubated at 28° C. with vigorous agitation. When the cells reached log phase (OD₅₅₀ 0.5-0.8), the culture was chilled by gently swirling it in an ice-water bath and kept at 4° C. for all further steps. The cells were pelleted by centrifuging at 4000 g for 10 minutes at 4° C. in a prechilled rotor. The supernatant was discarded, 5-10 ml of ice-cold water added, and the cells pipetted gently up and down until no clumps remained using a wide-bore pipette. The suspension volume was adjusted to 500 ml with ice-cold water. Centrifugation, removal of supernatant and volume readjustment was repeated twice. After the first repetition the volume was adjusted to 250 ml and after the second to 50 ml. The cells were pelleted by centrifugation at 4000 g for 10 minutes at 4° C. in a prechilled rotor, and resuspended in 5 ml of 10% (v/v) ice-cold, sterile glycerol. 50 μl aliquots of cells were dispensed into microcentrifuge tubes and snap-freezed in liquid nitrogen, and stored at −70° C.

### Electroporation and Recovery

Competent cells were thawed on ice (50 μl per transformation) and plasmid DNA (1 μl of E. coli miniprep or 1-5 μg of CsCl-purified plasmid DNA) was added to the cells and mixed together on ice. The mixture was transferred to a prechilled electroporation cuvette and electroporation carried out. After electroporation, the cells were recovered and selected for using antibiotic for the T-DNA vector as is well known in the art.

### Vectors and Agrobacterium Hosts for Arabidopsis Transformation

Standard protocols are available and used for the transformation of Agrobacterium (such as CSH Protocols; 2006, doi:10.1101/pdb.ip29, which was adapted from “How to Transform Arabidopsis,” Chapter 5, in Arabidopsis by Detlef Weigel and Jane Glazebrook. Cold Spring Harbor Laboratory Press, Cold Spring Harbor, N.Y., USA, 2002; http://www.cshprotocols.org/cgi/content/full/protocols;2006/30/pdb.ip29#R4). Numerous T-DNA vectors (Table 4) are available and can be used depending upon the antibiotic resistance desired. A guide to a T-DNA vector has been described by Hellens et al. (Hellens R, Mullineaux P, Klee H. 2000b. A guide to Agrobacterium binary Ti vectors. Trends Plant Sci. 5: 446-451) incorporated herein by reference.

### PCR Analysis of Agrobacterium

Standard protocols are available and were used for the PCR analysis of Agrobacterium (such as CSH Protocols; 2006, doi:10.1101/pdb.prot4667, which was adapted from “How to Transform Arabidopsis,” Chapter 5, in Arabidopsis by Detlef Weigel and Jane Glazebrook. Cold Spring Harbor Laboratory Press, Cold Spring Harbor, N.Y., USA, 2002).

### Example 3

### Transformation of A. thaliana

A. thaliana was chosen as a model plant for this project based on the following characteristics: accumulates seed oil to 41%; close relationship to the commercially grown oleaginous crop canola (B. napus); short life cycle (˜6 weeks); ease of transformation and selection. Agrobacterium tumefaciens—mediated transformation of A. thaliana C-24 plants was performed using a floral dip method (Clough and Bent, 1998, The Plant Journal 16:735-743). T₁ seedlings were identified on the selection medium containing phosphinothricin, which severely disrupts nitrogen metabolism and photosynthetic carbon fixation in wild type plants and causes leave chlorosis and eventually plant death. Phosphinothricin resistant T₁ plants were grown individually to produce mature T₂ seeds for seed oil analysis.

Arabidopsis plants are grown until they are flowering. The transformed A. tumefaciens are spun down, resuspended to OD₅₀₀=0.8 in 5% Sucrose solution and used for floral dipping. Before dipping, Silwet L-77 was added to a concentration of 0.05% (500 ul/L) and mixed well. The above-ground parts of the plant were dipped in Agrobacterium solution for 2 to 3 seconds, with gentle agitation until a film of liquid coated the plant. The dipped plants were placed under a dome or cover for 16 to 24 hours to maintain high humidity, and grown normally until seeds matured, when watering was stopped, and dry seeds were harvested. Transformants were selected using antibiotic or herbicide selectable marker and putative transformants were grown. Alternative protocols are well known and may be used, such as, “In planta transformation of Arabidopsis” (CSH Protocols; 2006, doi:10.1101/pdb.prot4668, which was adapted from “How to Transform Arabidopsis,” Chapter 5, in Arabidopsis by Detlef Weigel and Jane Glazebrook. Cold Spring Harbor Laboratory Press, Cold Spring Harbor, N.Y., USA, 2002).

### Example 4

### T₁ Selection and Propagation

A methodology for identifying transformed Arabidopsis thaliana seedlings has been described (Harrison, S. J.; Mott, E. K.; Parsley, K.; Aspinall, S.; Gray, J. C. and Cottage, A. A rapid and robust method of identifying transformed Arabidopsis thaliana seedlings following floral dip transformation, Plant Methods, 2006, 2, 19) and was utilized here, where screening was performed using antibiotics, such as kanamycin, or herbicides such as phosphinothricin and hygromycin B. As indicated above, selection of transformants from non-transformants requires the presence of markers, usually in the form of either antibiotic or herbicide resistance. Selection to, for example kanamycin, typically takes 7-10 days following germination (Bechtold, N.; Ellis, J.; Pelletier, G.: In planta Agrobacterium-mediated gene transfer by infiltration of adult Arabidopsis thaliana plants. C. R. Acad. Sci. Paris, Sciences de la vie/Life Science, 1993, 316, 1194-1199; Clough and Bent, 1998, Plant J. 16:735-743). A typical procedure for T₁ selection, as described in Harrison et al. is described below.

Seeds were surface sterilized by immersion in 70% (v/v) ethanol for 2 min, followed by immersion in 10% (v/v) sodium hypochlorite solution containing 8% available chlorine (Fisher Scientific, UK #S/5040/21) for 10 min. Seeds are then washed four times with sterile distilled water and sown onto 1% agar containing MS medium and kanamycin monosulphate at a concentration of 50 μg m1-1 (Melford Laboratories Ltd., Ipswich, UK #K0126), DL-phosphinothricin at a concentration of 50 μM (Melford Laboratories Ltd. #P01590250), or hygromycin B at a concentration of 15 μg m1-1 (Melford Laboratories Ltd. #H0125). Excess surface liquid was drained from the plates. Seeds were then stratified for 2 d in the dark at 4° C. After stratification seeds were transferred to a growth chamber (Multitron, Infors UK, Reigate, UK) and incubated for 4-6 h at 22° C. in continuous white light (120 μmol m−2 s1) in order to stimulate germination. The plates were then wrapped in aluminum foil and incubated for 2 d at 22° C. The foil removed and seedlings were incubated for 24-48 h at 22° C. in continuous white light (120 μmol m−2 s−1). Other methods are also available, such as the kanamycin or glufosinate ammonium based selection of the transformed Arabidopsis, as described in CSH Protocols; 2006, doi:10.1101/pdb.prot4669 and CSH Protocols; 2006, doi:10.1101/pdb.prot4670, which was adapted from “How to Transform Arabidopsis,” Chapter 5, in Arabidopsis by Detlef Weigel and Jane Glazebrook. Cold Spring Harbor Laboratory Press, Cold Spring Harbor, N.Y., USA, 2002; and are briefly described here below.

### Kanamycin Selection of Transformed Arabidopsis

An appropriate quantity of seeds were surface sterilized by soaking them in ethanol for 1 minute and then soaking in seed sterilization solution for an additional 10 minutes. The seeds are washed in four changes of H2O, and suspended in the appropriate volume of 0.1% agarose (5 ml/Petri dish or 100 mg of seed). The seed suspension is spread over the selection plates and allowed to dry a little so that the seeds do not float when the plate is moved. The plates are sealed with microporous tape and incubated at 4° C. to break dormancy. After 2 days, the plates are transferred to a plant tissue culture room with adequate light. After 7 days, the plates are checked for transformants. Transfer the transformants to soil and optionally verify by PCR that the transformants contain the construct of interest.

### Example 5

### Extraction and Analysis of Seed Oil and Fatty Acid (FA) Composition of T₂ Lines

Seed oil was extracted from mature T₂ A. thaliana seeds expressing ACBP using a hexane-isopropanol method (Hara and Radin, 1978, Anal Biochem. 90:420-426). Seed oil content was determined by gravimetric method in four replicates. In order to analyze FA composition of seed oil, FA present in seed oil extract in free or esterified form is methylated with HCl—Methanol and separated by gas chromatography (GC). FA profiles of seed oil from transgenic plants was compared to that of an A. thaliana controls: wild type seeds (WT) and seeds from the line that was transformed with ACBP construct, but segregated back to the WT genotype (Null Segr).

Addition of external standards (tripentadecanoylglycerol, 15:0-TAG; triheptadecanoylglycerol, 17:0-TAG) during seed oil extraction and FA methylation accounted for sample loss in those procedures. Also, addition of a precise amount of internal standard (methyl ester of eicosapentaenoic acid, 20:5 FAME) on GC column along with the sample let us estimate the total FA content (value very close to seed oil content) by GC.

Seeds of each sample were placed in a hexane-washed, hand-held, ground-glass homogenizer and boiled in 1 mL of isopropanol (80° C.) for 10 min. The seed was then cooled on ice for 5 min. Thereafter, 1 mL of hexane and 2 mL of 3:2 hexane:isopropanol (HIP) were added and the seed homogenized until completely pulverized. An additional 2 mL of 3:2 HIP was added and grinding continued. The slurry was transferred to a screw capped glass tube, and 2 mL of 3.3% (w/v) Na₂SO₄ added, capped, and shaken for 2 min. The tubes were then spun at 555 g for 2 min, and the upper organic phase transferred to a new hexane-washed screw-capped tube. The aqueous phase was re-extracted with 4 mL of 7:2 HIP, capped, and shaken for 2 min. The tubes were then spun again at 555 g for 2 min, and the upper organic phase added to the first extracted organic phase. The combined organic phases were evaporated to dryness in a heating block (37° C.) under a gentle nitrogen stream. To determine fatty acid methyl esters (FAMEs), 1.2 mL of HCl-methanol (1.5 M HCl in methanol made fresh) was added to the dried lipid and incubated at 100° C. for 1 h. Then, 1 mL of double distilled water was added to quench the transesterification reaction. The FAMEs were then extracted with 2 mL of hexane. The samples were centrifuged as above and the upper organic phase containing the FAMEs were transferred to a clean hexane-washed test tube. The aqueous phase was re-extracted with an addition 2 mL of hexane and centrifuged, and the resulting upper phase transferred and combined with the previously collected organic phase. The combined organic phase containing the FAMEs was then dried down completely in a heating block with a nitrogen stream. Finally, the FAMEs were solubilized in 1 mL of hexane and transferred to gas chromatography vials and capped.

FAMEs were analyzed on either an Agilent Technologies 6890N gas chromatograph or a Varian 3800 Gas Chromatograph equipped with an autosampler. FAMEs were separated and detected by flame ionization detection on a narrow-bore DB-23 column with constant flow 2 ml/min and a temperature program: 45° C. for 5 min, 45-175° C. at 13° C./min, hold at 175 for 37 min, 175-215° C. at 4° C./min, hold at 215° C. for 9 min, 215-240° C. at 5° C./min and hold at 240° C. for 5 min). Integration events detected and identified between 14 and 60 min were compared against a NuChek 463 or 502 gas-liquid chromatography standard. Alternatively, the method described by Focks can be used (Focks and Benning, 1998, Plant Physiol. 118: 91-101).

### Example 6

### Extraction and Analysis of Seed Oil and Fatty Acid (FA) Composition of a Subsequent Generation (T₃ Lines)

The seed oil from mature T3 A. thaliana seeds expressing ACBP was extracted and analyzed using the methods described in Example 5 above.

### Example 7

### Extraction and Analysis of Developing T₃ Embryos or Mature T₃ Seed for Transgene Expression

Developing embryos (excised embryos from siliques selected days after flowering (DAF) coincident with high triacylglycerol accumulation) or mature Arabidopsis seeds (25 mg) were ground in 0.5 mL of extraction buffer (0.4 M sucrose, 0.5 M NaCl, and 50 mM Tris-HCl, pH 8.0) and the total seed proteins (TSP) were solubilized with addition of 10% SDS (to a final concentration of 2% SDS) and boiled for 10 minutes. Thereafter total protein content was determined using BCA protein assay (Pierce, Rockford, Ill.). Total seed proteins were then analyzed by SDS-PAGE using standard protocols (Sambrook et al., 1989, Molecular Cloning, a Laboratory Manual, Cold Spring Harbor Laboratory Press) and stained with Coomassie Brilliant Blue R 250 or blotted for Western analysis.

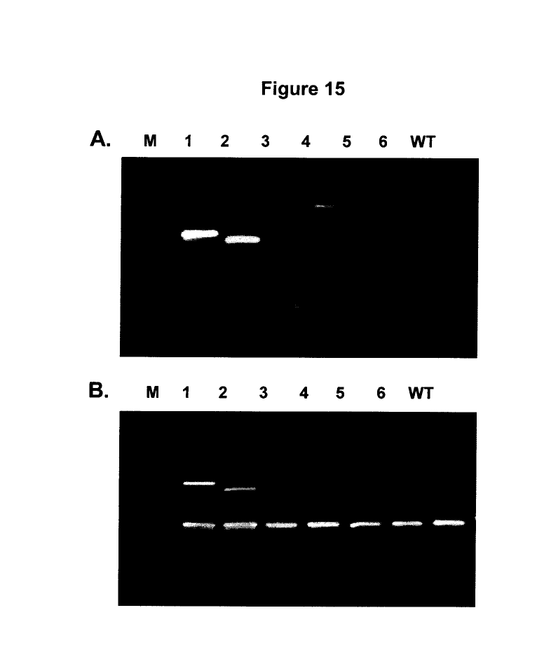

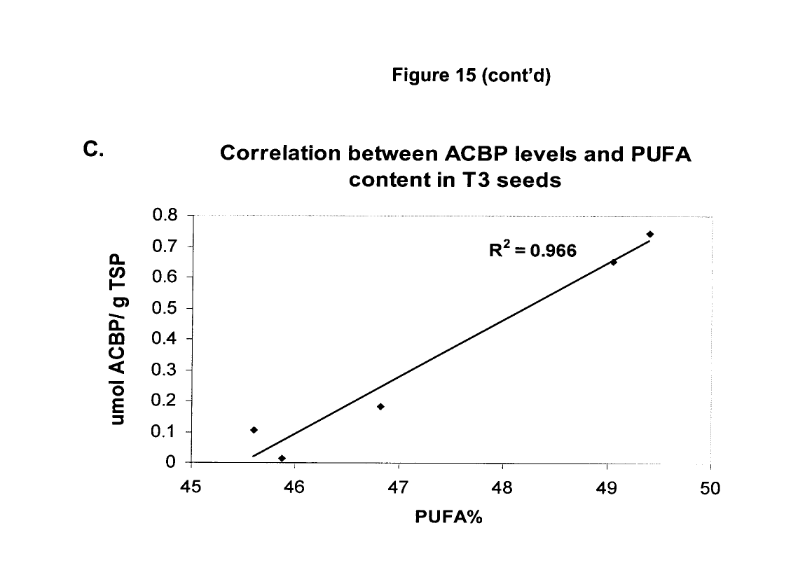

Samples were then loaded on discontinuous 10% SDS-PAGE gels on the basis of equal protein content for TSP analysis (FIG. 15) or equal volume for partitioned analysis of OB (oilbody), UND (undernatant) and P (pellet). Proteins were separated at 150 volts for approximately 1.5 hours and either Coomassie-stained or blotted onto PVDF membrane (Immobilon P, Millipor Corporation, Beford, Mass.) for Western blot analysis. Blotted samples were probed with polyclonal antibody directed against Brassica napus ACBP (Brown et al., 1998, Plant Physiol. Biochem. 36:629-635) or monoclonal antibody directed against Arabidopsis thaliana 18 kDa oleosin (antibodies were made by SemBioSys Genetics Inc.). ACBP was detected using secondary donkey anti-rabbit IRDye® 800 CW (LiCor Biosciences, Lincoln, Nebr.) and analyzed using the Odyssey Infrared Imaging System (LiCor Biosciences, Lincoln, Nebr.). The 18 kDa oleosin or oleosin fusion products was detected using secondary goat anti-mouse IRDyee 800 CW (LiCor Biosciences, Lincoln, Nebr.) and analyzed using the Odyssey Infrared Imaging System (LiCor Biosciences, Lincoln, Nebr.). The 18 kDa oleosin monoclonal antibody was used in three capacities, one to detect transgene products where the ACBP was fused to oleosin, secondly to ensure equal loading of the protein occurred, and thirdly as an internal standard to determine transgene expression levels (based on the determination that the endogenous 18 kDa oleosin expresses at a level equivalent to 1.5% of the total seed protein in mature Arabidopsis seeds; Nykiforuk et al., 2006).

FIG. 15 (A) revealed all transgene products were expressed in mature T2 recombinant seed (including construct 8; D9-ACBP, lane 6, expressed albeit at low levels) and equal loading of protein was obtained for all samples as revealed by the same level of intensity of the lower band in FIG. 15 (B). When analysis was performed using T3 developing seed at 16 DAF (FIG. 15 (C)), using the endogenous 18 kDa oleosin expression level as an internal standard, a strong positive correlation between the transgenic ACBP levels and final seed oil PUFA content existed when the Construct 7 (ACBP) data point was excluded. The fact that transgenic lines with Construct 7 (ACBP) had the highest PUFA levels in seed oil suggests that factors including seed-specific expression (versus constitutive expression), ACBP stability (fusion versus non-fusion), cellular localization (OB or ER associated versus cytosolic) and perhaps localized concentration may all contribute in part to the observed increases in PUFA and/or oil content.

### Example 8

### Expression, Purification and Characterization of the Recombinant B. napus Acyl CoA Binding Protein

B. napus acyl CoA binding protein cDNA may be cloned into pET vector (Novagen, Madison, Wis.) to produce (His)₆-tagged recombinant protein (rACBP) in Escherichia coli system. (His)₆-rACBP may be purified by immobilized nickel ion chromatography, followed by further purification using gel filtration chromatography. The purified rACBP may be used in binding studies with radiolabelled (¹⁴C) acyl-CoA species common for oilseed crops (18:1-, 18:2-, 18:3-CoAs) and unusual acyl-CoAs desirable for engineering in plants (20:5-CoA-eicosapentaenoic acid, EPA; 22:6-CoA-docosahexaenoic acid, DHA). Acyl-CoA binding properties of rACBP may be assessed using a Lipidex 1000 column, which is routinely used in acyl-CoA binding assays (Engeseth et al., 1996, Archives of Biochemistry and biophysics 331:55-62; Chye et al., 2000, Plant Mol. Biol. 44:711-721; Leung et al., 2004, Plant Mol. Biol. 55:297-309). Lipidex1000 is a relatively simple binding assay that allows determination of binding constants for acyl-CoA binding to acyl CoA binding protein (Rasmussen et al., 1990, Biochem. J. 265:849-855). It should be noted, however, that the Lipidex competition assay does not give absolute K_{d} value (dissociation constant) but rather binding relative to the affinity of Lipidex1000 (Mandrup et al., 1991, Biochem. J. 276:817-823). This complication is imposed by an exceptionally high affinity of acyl CoA binding protein toward acyl-CoAs, which makes acyl CoA binding protein able to extract acyl-CoA esters bound to the Lipidex column (Rosendal et al., 1993, Biochem. J. 290:321-326). However, for comparison of relative binding affinity the method is acceptable.

### Example 9

### In Vitro Acyltransferase Assays

Microsomal membranes from the microspore derived cell suspension cultures of B. napus may be isolated by differential centrifugation and used as a source of acyltransferase activity in the assays with rACBP in the reaction mixture at different concentrations. The effect of plant and animal rACBP on acyltransferase activities in vitro has previously been studied and appeared to be dependent on acyl CoA binding protein:acyl-CoA ratio in the reaction mixture (Brown et al., 1998, Plant Physiol. Biochem. 36:629-635; Abo-Hashema et al., 2001, The International Journal of Biochemistry & Cell Biology 33:807-815; Chao et al., 2003, J. Lipid Res 44:72-83). It has been proposed that acyl CoA binding protein can transport and donate acyl-CoAs for glycerolipid synthesis, and that the acyl-CoA-acyl CoA binding protein complex is preferred over free acyl-CoAs by some acyltransferases (Rasmussen et al., 1994, Biochem. J. 299:165-170; Fyrst et al., 1995, Biochem. J. 306:793-799). Selectivity studies with acyltransferases of the Kennedy pathway are performed to determine if acyl CoA binding protein can affect the enzyme preference for different species of acyl-CoAs for esterification of the glycerol backbone. Reaction mixtures may include equimolar quantities of radiolabelled (¹⁴C) endogenous acyl-CoAs and/or unusual acyl-CoA esters. Following the reaction, the appropriate radiolabeled enzyme product may be isolated by thin layer chromatography (TLC) and the constituent FAs converted to fatty acid methyl esters. A GC with radiodetector may be used to identify which radiolabelled acyl-CoA is predominantly incorporated into TAG by the acyltransferase of interest in the presence of rACBP.

### Example 10

### Fatty Acid Analysis

Analysis of A. thaliana T₂ lines showed 5 out of 70 samples showed statistically significant increases in oil content. Analysis of those 5 lines by gravimetric analysis showed increases of oil content from 1.97 to 7.72% weight difference, while analysis by gas chromatography showed increased oil content from 1.33 to about 9.4% weight difference (Table 5).

Considerable variation in the range and direction of the changes in FA composition was observed in T₂ seeds, because each T₂ line represents a different insertion event, which may have a significant positional effect on the levels of the transgene expression (Table 6). The major trend in the transgenic seeds was an increase in levels of PUFAs comparing to the controls (WT, Null Segr and constitutive lines). T; lines transformed with 5 out of 8 constructs with PhaP promoter (ACBP-1>B82-Oleosin-ACBP-1>ACBP-1-Oleosin>OleosinH3P-ACBP-1>ACBP-1-KDEL) showed significant increase in PUFA comparing to WT (up to 4.8±0.13% weight abs.−maximum difference between the mean PUFA % of T₂ line and WT±std error of the difference). Increase in PUFA in seeds transformed with those constructs was mostly at expense of MUFA (Table 7). Lines transformed with ACBP-1 and ACBP-1-KDEL expressed under regulation of constitutive promoter 35S had a decreased PUFA and increased MUFA content comparing to the controls. Saturated fatty acids (SFA) were slightly reduced in seed oil in T₂ lines with constructs under the regulation of the phaseolin promoter (PhaP) in OleosinH3P-ACBP-1, ACBP-1, D9-ACBP-1 and under the regulation of the constitutive 35S-ACBP and significant increased in lines expressing PhaP-B82-Oleosin-ACBP. The observed changes in composition of FA classed in seed oil in transgenic plants was due to the presence of the transgene, since composition of the Null Segr seeds reversed back to the WT phenotype after loosing the insertion.

More detailed profile of the FA composition of T₂ seeds shows that increase in PUFA in lines with PhaP constructs was mainly due to increase in 18:2, and in construct PhaP-ACBP-1 also in 18:3 (Table 8). Lines transformed with seed preferred construct other than ACBP-1 had a small decrease in 18:3. The increase in PUFA content in seed oil appeared to happen at the expense of MUFA, particularly 20:1. Decrease in SFA was due to reduced amount of 18:0, and in construct PhaP-ACBP-1 also 16:0.

Analysis of the 10 T₃ lines per T₂ line, selected in the previous round of the seed oil analysis (4 T₂ lines per construct) provided us with more statistically reliable data that confirmed our previous findings. T₃ seeds obtained from T₂ lines transformed with construct ACBP-1-Oleosin, OleosinH3P-ACBP-1 and ACBP-1 with PhaP show significant increase in PUFA (an mean increase of up to 3.06% weight abs. difference) comparing to WT (Table 9). Just as in T₂ seeds, an increase in levels of PUFAs in T₃ seeds was due to an increase in 18:2 (Table 10). Changes in MUFA composition that included a decrease in 20:1 for all constructs in this data set, except for D9-ACBP-1, and an increase in 18:1 for constructs expressed as D9 Scfv fusions, resulted in very little changes in total MUFA as a group. The decrease in SFA observed in lines expressing constructs ACBP-1-Oleosin, D9-ACBP-1-KDEL, ACBP-1 and D9-ACBP-1 was mainly attributed to a decrease in 18:0, and also 16:0 for construct ACBP-1. Comparing data from two generations of the transgenic seeds expressing ACBP, it can be seen that the magnitude of changes in FA composition in T₃ seeds is more subtle compared to T₂ seeds data. Results obtained from both data sets point out the major effect of the ACBP transgene on seed oil composition, which is the increase in 18:2 and decrease in 20:1.

### Example 11

### Seed Preferred Expression Constructs Resulting in Increased PUFA Content and Seed Oil Content

The use of seed preferred promoters to drive the over-expression of ACBP in the configurations outlined in Examples 1, FIG. 3 or FIG. 4, could be used to alter the fatty acid content and/or seed oil content of oilseeds. Seed preferred promoters containing a number of conserved or consensus motifs in the sequences upstream (5′) of the transcriptional start site of cDNA encoding for ACBP, in addition to basal elements required for transcription initiation, are anticipated to result in elevated expression during oilseed development when triacylglycerol biosythesis and TAG bioassembly are occurring at high levels. As shown in the examples above the over expression of ACBP in a temporal and tissue specific manner during oilseed development resulted in significant changes in fatty acid profile and seed oil content. Further manipulation of fatty acid content may be achieved by employing ACBPs conferring selectivity for different acyl-CoAs expressed in this manner. In the context of the current disclosure these additional seed preferred expression cassettes could be used:

1. B. napus acyl CoA binding protein cDNA (encoding for ACBP-1) fusion in frame with A. thaliana 18 kDa oleosin gene at the C′-terminus under the seed preferred control of an oleosin promoter:terminator.
2. B. napus acyl CoA binding protein cDNA (encoding for ACBP-1) fusion in frame with A. thaliana 18 kDa oleosin gene at the C′-terminus under the seed preferred control of an linin promoter:terminator.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Acyl CoA Binding Proteins
Acyl Co A Binding ProteinMotif
(Amino Acid Sequence Identifier) {Nucleic Acid Sequence Identifier}

| | |
|---|---|
| (AAA34384) Saccharomyces cerevisiae | (O04066) Ricinus communis (castor bean) |
| acyl-CoA-binding protein | acyl CoA binding protein |
| (NP_001075582) Oryctolagus cuniculus | (P45882) Anas platyrhynchos Acyl-CoA- |
| (rabbit) acyl CoA binding protein | binding protein (ACBP) (Diazepam-binding |
| (AAK98608) Oryctolagus cuniculus (rabbit) | inhibitor) (DBI) (Endozepine) (EP) |
| acyl CoA binding protein | (NP_001037022) Bombyx mori (domestic |
| (XP_001348923) Plasmodium falciparum | silkworm) acyl-CoA binding protein |
| 3D7 | (NP_001037023) Bombyx mori (domestic |
| (XP_001024105) Tetrahymena thermophila | silkworm), acyl-CoA binding protein |
| SB210 acyl CoA binding protein | (ZP_01735531) Marinobacter sp. ELB17 |
| (XP_001012898) Tetrahymena thermophila | Acyl-CoA-binding protein |
| SB210 acyl CoA binding protein | (ZP_01721035) Algoriphagus sp. PR1 |
| (XP_001011628) Tetrahymena thermophila | acyl-CoA-binding protein |
| SB210 acyl CoA binding protein | (EBA01487) Marinobacter sp. ELB17 acyl- |
| (EAR92653) Tetrahymena thermophila | CoA-binding protein |
| SB210 acyl CoA binding protein | (EAZ79764) Algoriphagus sp. PR1 acyl- |
| (EAR91383) Tetrahymena thermophila | CoA-binding protein |
| SB210 acyl CoA binding protein | (P57752) Arabidopsis thaliana (thale cress) |
| (AAN37362) Plasmodium falciparum 3D7 | acyl-CoA-binding protein |
| acyl CoA binding protein | (P12026) Sus scrofa (pig) Acyl-CoA- |
| (P11030) Rattus norvegicus (Norway rat) | binding protein (ACBP) (Diazepam-binding |
| Acyl-CoA-binding protein (ACBP) | inhibitor) (DBI) (Endozepine) (EP) |
| (Diazepam-binding inhibitor) (DBI) | [Contains: DBI(32-86)] |
| (Endozepine) (EP) [Contains: | (Q8WN94) Oryctolagus cuniculus (rabbit) |
| Triakontatetraneuropeptide (TTN); | Acyl-CoA-binding protein (ACBP) |
| Octadecaneuropeptide (ODN)]. | (Diazepam-binding inhibitor) (DBI) |
| (NP_001033088) Mus musculus (house | (Endozepine) (EP) |
| mouse) diazepam biding inhibitor isoform 1 | (Q9TQX6) Canis familiaris (dog) Acyl-CoA- |
| (NP_031856) Mus musculus (house | binding protein (ACBP) (Diazepam-binding |
| mouse) diazepam biding inhibitor isoform 2 | inhibitor) (DBI) (Endozepine) (EP) |
| (P07107) Bos taurus (cattle) Acyl-CoA- | (P31786) Acyl-CoA-binding protein (ACBP) |
| binding protein (ACBP) (Diazepam-binding | (Diazepam-binding inhibitor) (DBI) |
| inhibitor) (DBI) (Endozepine) (EP). | (Endozepine) (EP) Mus musculus (house |
| (NP_114054) Rattus norvegicus (Norway | mouse) |
| rat) diazepam binding inhibitor | (P07108) Homo sapiens (human) Acyl- |
| (AAF78043) Bombyx mori (domestic | CoA-binding protein (ACBP) (Diazepam- |
| silkworm) acyl CoA binding protein | binding inhibitor) (DBI)(Endozepine) (EP) |
| (AAF78042) Bombyx mori (domestic | (Q9PRL8) Gallus gallus (chicken) Acyl- |
| silkworm) acyl CoA binding protein | CoA-binding protein (ACBP) (Diazepam- |
| (P31787) Saccharomyces cerevisiae acyl | binding inhibitor) (DBI) |
| CoA binding protein | (O22643) Fritillaria agrestis Acyl-CoA- |
| (P82934) Chaetophractus villosus (large | binding protein (ACBP) |
| hairy armadillo) acyl CoA binding protein | (NP_001040308) Bombyx mori (domestic |
| (Q39315) Brassica napus (rape) acyl CoA | silkworm) acyl-CoA binding protein |
| binding protein | (ABE72959) Jatropha curcas acyl-CoA- |
| (Q39779) Gossypium hirsutum (upland | binding protein |
| cotton) acyl CoA binding protein | (CAL67654) Gramella forsetii KT003 acyl- |
| (ABD65295) Cryptosporidium parvum acyl- | CoA-binding protein |
| CoA-binding protein | (YP_72656) Ralstonia eutropha H16 acyl- |
| (ZP_0099724) Janibacter sp. HTCC2649 | CoA-binding protein |
| acyl-CoA-binding protein | (CAJ9321) Ralstonia eutropha H16 acyl- |
| (EAP97105) Janibacter sp. HTCC2649 | CoA-binding protein |
| acyl-CoA-binding protein | (XP_646321) Dictyostelium discoideum |
| (AAA1224) Rattus norvegicus (Norway rat) | AX4 acyl-CoA-binding protein |
| acyl-CoA-binding protein | (YP_67936) Cytophaga hutchinsonii ATCC |
| (CAA70200) Ricinus communis (castor | 33406 acyl-CoA-binding protein |
| bean) acyl-CoA-binding protein | (ABG60493) Cytophaga hutchinsonii ATCC |
| (CAA54390) Brassica napus (rape) acyl- | 33406 acyl-CoA-binding protein |
| CoA binding protein | (XP_29344) Trypanosoma brucei |
| (AAS2090) Hyacinthus orientalis acyl-CoA- | TREU927 acyl-CoA-binding protein |
| binding protein | (XP_16412) Trypanosoma cruzi strain CL |
| (AAT0701) Hyacinthus orientalis | Brener acyl-CoA-binding protein |
| (AAQ4320) Gossypium barbadense (sea- | (XP_13234) Trypanosoma cruzi strain CL |
| island cotton) acyl-CoA-binding protein | Brener acyl-CoA-binding protein |
| (AAP2942) Tropaeolum majus (nasturtium) | (XP_10104) Trypanosoma cruzi strain CL |
| acyl-CoA-binding protein | Brener acyl-CoA-binding protein |
| (AAF0323) Arabidopsis thaliana (thale | (XP_954639) Theileria annulata strain |
| cress) acyl-CoA-binding protein | Ankara acyl-CoA-binding protein |
| (BAB597) Panax ginseng acyl-CoA-binding | (EAL72679) Dictyostelium discoideum AX4 |
| protein | acyl-CoA-binding protein |
| (AAF75257) Trypanosoma brucei acyl- | (YP_431917) Hahella chejuensis KCTC |
| CoA-binding protein | 2396 acyl-CoA-binding protein |
| (AAB67736) Gossypium hirsutum (upland | (YP_103223) Burkholderia mallei ATCC |
| cotton) acyl-CoA-binding protein | 23344 acyl-CoA-binding protein |
| (AAM2250) Oryza sativa (japonica cultivar- | (ABD3611) Bombyx mori (domestic |
| group) acyl-CoA-binding protein | silkworm) acyl-CoA-binding protein |
| (ABN91) Burkholderia pseudomallei 1106a | (YP_442524) Burkholderia thailandensis |
| acyl-CoA-binding protein | E264 acyl-CoA-binding protein |
| (ABN3142) Burkholderia pseudomallei 66 | (ABC3043) Burkholderia thailandensis |
| acyl-CoA-binding protein | E264 acyl-CoA-binding protein |
| (ABO0553) Burkholderia mallei NCTC | (AAF09755) Deinococcus radiodurans R1 |
| 10247 acyl-CoA-binding protein | acyl-CoA-binding protein |
| (ABI14372) Pfiesteria piscicida acyl-CoA- | (ABC27492) Hahella chejuensis KCTC |
| binding protein | 2396 acyl-CoA-binding protein |
| (YP_001029163) Burkholderia mallei | (NP_29390) Deinococcus radiodurans R1 |
| NCTC 10229 acyl-CoA-binding protein | acyl-CoA-binding protein |
| (EAY64349) Burkholderia cenocepacia | (AAW700) Dictyostelium discoideum acyl- |
| PC14 acyl-CoA-binding protein | CoA-binding protein |
| (YP_69234) Alcanivorax borkumensis SK2 | (CAD1900) Stigmatella aurantiaca acyl- |
| acyl-CoA-binding protein | CoA-binding protein |
| (EAY29965) Microscilla marina ATCC | (AAU4124) Burkholderia mallei ATCC |
| 23134 acyl-CoA-binding protein | 23344 acyl-CoA-binding protein |
| (ZP_016753) Microscilla marina ATCC | (AAD0342) Arabidopsis thaliana (thale |
| 23134 acyl-CoA-binding protein | cress) acyl-CoA-binding protein |
| (YP_993401) Burkholderia mallei SAVP1 | (AAB651) Fritillaria agrestis acyl-CoA- |
| acyl-CoA-binding protein | binding protein |
| (CAL16562) Alcanivorax borkumensis SK2 | (YP_00106671) Burkholderia pseudomallei |
| acyl-CoA-binding protein | 1106a acyl-CoA-binding protein |
| (YP_00100909) Burkholderia mallei NCTC | acyl CoA binding protein, putative |
| 10247 acyl-CoA-binding protein | (EAA21013) Plasmodium yoelii yoelii Acyl |
| (YP_00105949) Burkholderia pseudomallei | CoA binding protein, putative |
| 66 acyl-CoA-binding protein | (AAK00406) Arabidopsis thaliana putative |
| (ABN02373) Burkholderia mallei NCTC | Acyl CoA binding protein |
| 10229 acyl-CoA-binding protein | (AAG4147) Arabidopsis thaliana putative |
| (ABM52014) Burkholderia mallei SAVP1 | Acyl CoA binding protein |
| acyl-CoA-binding protein | (XP_001264530) Neosartorya fischeri |
| (YP_62721) Gramella forsetii KT003 acyl- | NRRL 11 Acyl CoA binding protein, |
| CoA-binding protein | putative |
| (YP_334003) Burkholderia pseudomallei | (XP_001260577) Neosartorya fischeri |
| 1710b acyl-CoA-binding protein | NRRL 11 Acyl CoA binding protein family |
| (ABA50922) Burkholderia pseudomallei | (NP_001073332) Homo sapiens diazepam |
| 1710b acyl-CoA-binding protein | binding inhibitor isoform 2 |
| (NP_200159) Arabidopsis thaliana (thale | (NP_001073331) Homo sapiens diazepam |
| cress) acyl-CoA-binding protein | binding inhibitor isoform 3 |
| (XP_001275397) Aspergillus clavatus | (NP_06543) Homo sapiens diazepam |
| NRRL 1 acyl-CoA-binding protein | binding inhibitor isoform 1 |
| (XP_00126902) Aspergillus clavatus NRRL | (P6167) Saccharomyces pastorianus |
| 1 putative Acyl CoA binding protein | Acyl-CoA-binding protein 2 |
| (XP_001347301) Plasmodium falciparum | (1HBK_A) Plasmodium falciparum (malaria |
| 3D7 putative Acyl CoA binding protein | parasite P. falciparum) |
| (XP_001347300) Plasmodium falciparum | Chain A, Acyl-Coa Binding Protein |
| 3D7 putative Acyl CoA binding protein | (2CQU_A) Homo sapiens (human)Chain A, |
| (EAW22633) Neosartorya fischeri NRRL 11 | Solution Structure Of Rsgi Ruh-045, A |
| putative Acyl CoA binding protein | Human Acyl-Coa Binding Protein. |
| (EAW160) Neosartorya fischeri NRRL 11 | (CAJ00737) Mus musculus (house mouse) |
| Acyl CoA binding protein family | diazepam binding inhibitor, splice form 1b |
| (EAW13971) Aspergillus clavatus NRRL 1 | (1HB_C) Bos taurus (cattle) Chain C, |
| Acyl CoA binding protein family | Structure Of Bovine Acyl-Coa Binding |
| (EAW07602) Aspergillus clavatus NRRL 1 | Protein In Tetragonal Crystal Form. |
| Acyl CoA binding protein, putative | (1HB_B) Bos taurus (cattle) |
| (ABF94919) Oryza sativa (japonica | Chain B, Structure Of Bovine Acyl-Coa |
| cultivar-group) Acyl CoA binding protein, | Binding Protein In Tetragonal Crystal Form. |
| expressed | (1HB_A) Bos taurus (cattle) Chain A, |
| (ABF9491) Oryza sativa (japonica cultivar- | Structure Of Bovine Acyl-Coa Binding |
| group) | Protein In Tetragonal Crystal Form |
| Acyl CoA binding protein, expressed | (1HB6_A) Bos taurus (cattle) |
| (AAM6563) Arabidopsis thaliana (thale | Chain A, Structure Of Bovine Acyl-Coa |
| cress) Acyl CoA binding protein, putative | Binding Protein In Orthorhombic Crystal |
| (EAN33311) Theileria parva acyl CoA | Form |
| binding protein, putative | (CAJ0790) Leishmania major acyl-coa |
| (EAL93401) Aspergillus fumigatus Af293 | binding protein, putative |
| Acyl CoA binding protein family | (YP_170460) Francisella tularensis subsp. |
| (CAE47956) Aspergillus fumigatus acyl | tularensis SCHU S4 fusion product of 3- |
| CoA binding protein, putative | hydroxacyl-CoA dehydrogenase and acyl- |
| (AAG50714) Arabidopsis thaliana (thale | CoA-binding protein |
| cress) Acyl CoA binding protein, putative | (P616) Saccharomyces monacensis Acyl- |
| (AAN35214) Plasmodium falciparum 3D7 | CoA-binding protein 2 (ACBP type 2) |
| acyl CoA binding protein, putative | (YP_9472) Acidovorax sp. JS42 acyl-coA- |
| (AAN35213) Plasmodium falciparum 3D7 | binding protein, ACBP |
| (ABM40706) Acidovorax sp. JS42 acyl- | (XP_44351) Trypanosoma brucei |
| coA-binding protein, ACBP | TREU927 acyl-CoA binding protein, |
| (ABM306) Polaromonas naphthalenivorans | putative |
| CJ2 acyl-coA-binding protein, ACBP | (ABK0797) Burkholderia cenocepacia |
| (ABM31371) Acidovorax avenae subsp. | HI2424 |
| citrulli AAC00-1 acyl-coA-binding protein, | acyl-coA-binding protein, ACBP |
| ACBP | (CAJ02692) Leishmania major |
| (YP_960990) Marinobacter aquaeolei VT | acyl-CoA binding protein, putative |
| acyl-coA-binding protein, ACBP | (YP_667592) Francisella tularensis subsp. |
| (ABM2003) Marinobacter aquaeolei | tularensis FSC 19 fusion product of 3- |
| VTacyl-coA-binding protein, ACBP | hydroxacyl-CoA dehydrogenase and acyl- |
| (ZP_01579306) Delftia acidovorans SPH-1 | CoA-binding protein |
| acyl-coA-binding protein, ACBP | (CAL09546) Francisella tularensis subsp. |
| (ZP_01572045) Burkholderia multivorans | tularensis FSC 19 |
| ATCC 17616acyl-coA-binding protein, | fusion product of 3-hydroxacyl-CoA |
| ACBP | dehydrogenase and acyl-CoA-binding |
| (ZP_0156221) Burkholderia cenocepacia | protein |
| MC0-3 | (NP_49432) Arabidopsis thaliana (thale |
| acyl-coA-binding protein, ACBP | cress) acyl-CoA binding |
| (EAV76242) Delftia acidovorans SPH-1 | (NP_194507) Arabidopsis thaliana (thale |
| acyl-coA-binding protein, ACBP | cress) ACBP2 (ACYL-COA BINDING |
| (EAV63973) Burkholderia multivorans | PROTEIN ACBP 2) |
| ATCC 17616 acyl-coA-binding protein, | (NP_194154) Arabidopsis thaliana (thale |
| ACBP | cress) |
| (EAV59593) Burkholderia cenocepacia | acyl-CoA binding |
| MC0-3 | (NP_174462) Arabidopsis thaliana (thale |
| acyl-coA-binding protein, ACBP | cress) acyl-CoA binding |
| (ZP_0153113) Roseiflexus castenholzii | (YP_62569) Burkholderia cenocepacia AU |
| DSM 13941 acyl-coA-binding protein, | 1054 acyl-coA-binding protein, ACBP |
| ACBP | (ABF096) Burkholderia cenocepacia AU |
| (ZP_0151210) Comamonas testosteroni | 1054 |
| KF-1 acyl-coA-binding protein, ACBP | acyl-coA-binding protein, ACBP |
| (ZP_01516603) Chloroflexus aggregans | (YP_60366) Deinococcus geothermalis |
| DSM 945 | DSM 11300 acyl-coA-binding protein, |
| acyl-coA-binding protein, ACBP | ACBP |
| (ZP_015127) Burkholderia phytofirmans | (ABF44697) Deinococcus geothermalis |
| PsJN acyl-coA-binding protein, ACBP | DSM 11300 acyl-coA-binding protein, |
| (ZP_01504071) Burkholderia phymatum | ACBP |
| STM15 | (CAG46163) Francisella tularensis subsp. |
| acyl-coA-binding protein, ACBP | tularensis SCHU S4 fusion product of 3- |
| (EAV2706) Roseiflexus castenholzii DSM | hydroxacyl-CoA dehydrogenase and acyl- |
| 13941acyl-coA-binding protein, ACBP | CoA-binding protein |
| (EAV17679) Comamonas testosteroni KF-1 | (AAB3126) Anas platyrhynchos |
| acyl-coA-binding protein, ACBP | ACBP/DBI |
| (EAV0969) Chloroflexus aggregans DSM | (CAJ00736) Homo sapiens (human) |
| 945 | diazepam binding inhibitor, splice form 1c |
| acyl-coA-binding protein, ACBP | (YP_1071) Burkholderia pseudomallei |
| (EAV0253) Burkholderia phytofirmans | K96243 putative acyl-CoA-binding protein |
| PsJN acyl-coA-binding protein, ACBP | (YP_93727) Polaromonas |
| (YP_35690) Burkholderia cenocepacia | naphthalenivorans CJ2 |
| HI2424 acyl-coA-binding protein, ACBP | acyl-coA-binding protein, ACBP |
| (YP_969145) Acidovorax avenae subsp. | (BAF11442) Oryza sativa (japonica |
| citrulli AAC00-1 | cultivar-group) Os03g0243600 |
| acyl-coA-binding protein, ACBP | (P4221) Drosophila melanogaster (fruit fly) |
| (ZP_01557447) Burkholderia ambifaria | Acyl-CoA-binding protein homolog (ACBP) |
| MC40-6 | (Diazepam-binding inhibitor homolog) |
| acyl-coA-binding protein, ACBP | (DBI). |
| (EAV499) Burkholderia ambifaria MC40-6 | (P453) Rana ridibunda (marsh frog) Acyl- |
| acyl-coA-binding protein, ACBP | CoA-binding protein homolog (ACBP) |
| (EAU95939) Burkholderia phymatum | (Diazepam-binding inhibitor homolog) |
| STM15 acyl-coA-binding protein, ACBP | (DBI). |
| (YP_77396) Burkholderia cepacia AMMD | (ZP_01663657) Ralstonia pickettii 12J |
| acyl-coA-binding protein, ACBP | putative acyl-CoA-binding protein |
| (ABI7634) Burkholderia cepacia AMMD | (P1625) Digitalis lanata Acyl-CoA-binding |
| acyl-coA-binding protein, ACBP | protein 2 |
| (CAH361) Burkholderia pseudomallei | (ACBP 2) |
| K96243 | (P3124) Manduca sexta (tobacco |
| putative acyl-CoA-binding protein | hornworm) |
| (ZP_01734494) Flavobacteria bacterium | Acyl-CoA-binding protein homolog (ACBP) |
| BAL3 hypothetical protein FBBAL3_09114 | (Diazepam-binding inhibitor homolog) |
| (EAZ95136) Flavobacteria bacterium BAL3 | (DBI). |
| hypothetical protein FBBAL3_09114 | (2FDQ_C) Chaetophractus villosus (large |
| (CAB09005) Caenorhabditis elegans | hairy armadillo) Chain C, Crystal Structure |
| Hypothetical protein Y41E3.7a | Of Acbp From Armadillo Harderian Gland |
| (Q20507) Caenorhabditis elegans | (2FDQ_B) Chaetophractus villosus (large |
| Acyl-CoA-binding protein homolog 3 | hairy armadillo) |
| (ACBP-3) | Chain B, Crystal Structure Of Acbp From |
| (O0105) Caenorhabditis elegans Acyl-CoA- | Armadillo Harderian Gland |
| binding protein homolog 1 (ACBP-1) | (2FDQ_A) Chaetophractus villosus (large |
| (Diazepam-binding inhibitor homolog) (DBI) | hairy armadillo) |
| (BAF16206) Oryza sativa (japonica | Chain A, Crystal Structure Of Acbp From |
| cultivar-group) Os04g061900 | Armadillo Harderian Gland |
| (EAQ40949) Polaribacter dokdonensis | (CAG9732) Debaryomyces hansenii |
| MED152 | CBS767 unnamed protein product |
| hypothetical protein MED152_12964 | (CAB91232) Neurospora crassa related to |
| (EAQ39242) Dokdonia donghaensis | endozepine |
| MED134 hypothetical protein | (AAK307) Arabidopsis thaliana (thale |
| MED134_01445 | cress)putative membrane-bound acyl-CoA |
| (BAF22976) Oryza sativa (japonica | binding protein isoform 2 |
| cultivar-group) | (CAB1427) Arabidopsis thaliana (thale |
| Os0g016200 | cress) |
| (BAF1525) Oryza sativa (japonica cultivar- | putative acyl-CoA binding protein |
| group) | (CAB79333) Arabidopsis thaliana (thale |
| Os06g0115300 | cress) |
| (BAF13733) Oryza sativa (japonica | putative protein |
| cultivar-group) | (CAB4505) Arabidopsis thaliana (thale |
| Os03g035600 | cress) putative protein |
| (BAF12450) Oryza sativa (japonica | (CAB43966) Arabidopsis thaliana (thale |
| cultivar-group) | cress) |
| Os03g0576600 | putative acyl-CoA binding protein |
| (NP_001061062) Oryza sativa (japonica | (CAA65396) Rattus norvegicus (Norway |
| cultivar-group) | rat) |
| Os0g016200 | multifunctional acyl-CoA-binding protein |
| (NP_001056611) Oryza sativa (japonica | (ZP_01122235) Robiginitalea biformata |
| cultivar-group) | HTCC2501 hypothetical protein |
| Os06g0115300 | RB2501_02340 |
| (NP_001054292) Oryza sativa (japonica | (ZP_0111795) Polaribacter irgensii 23-P |
| cultivar-group) Os04g061900 | phosphatidylserine decarboxylase |
| (NP_00105119) Oryza sativa (japonica | (EAR14226) Robiginitalea biformata |
| cultivar-group) | HTCC2501 hypothetical protein |
| Os03g035600 | RB2501_02340 |
| (NP_001050536) Oryza sativa (japonica | (EAR12404) Polaribacter irgensii 23-P |
| cultivar-group) | phosphatidylserine decarboxylase |
| Os03g0576600 | (AAH60792) Homo sapiens (human) |
| (NP_00104952) Oryza sativa (japonica | ACBD3 protein |
| cultivar-group) | (AAH41143) Homo sapiens (human) |
| Os03g0243600 | ACBD4 protein |
| (BAB32079) Mus musculus (house mouse) | (AAH29164) Homo sapiens (human) |
| unnamed protein product | ACBD4 protein |
| (BAB2736) Mus musculus (house mouse) | (AAH2537) Mus musculus (house mouse) |
| unnamed protein product | Acbd6 protein |
| (BAB22124) Mus musculus (house mouse) | (AAH0193) Mus musculus (house mouse) |
| unnamed protein product | Peci protein |
| (EAQ50933) Leeuwenhoekiella blandensis | (AAB31937) Saccharomyces bayanus |
| MED217 hypothetical protein | acyl-coA-binding protein type 2, ACBP type |
| MED217_15360 | 2 = type 2 |
| (CAA69946) Saccharomyces monacensis | (AAB31936) Saccharomyces bayanus |
| ACB1 | acyl-coA-binding protein type 1, ACBP type 1 |
| (CAA6994) Saccharomyces pastorianus | (ZP_01059101) Leeuwenhoekiella |
| ACB1 type 2 | blandensis MED217 |
| (CAA69947) Saccharomyces pastorianus | hypothetical protein MED217_15360 |
| ACB1 type 1 | (ZP_01054200) Tenacibaculum sp. |
| (CAA69944) Saccharomyces cerevisiae | MED152 hypothetical protein |
| (baker's yeast) | MED152_12964 |
| ACB1 | (ZP_01050227) Cellulophaga sp. MED134 |
| (ZP_0135934) Roseiflexus sp. RS-1 | hypothetical protein MED134_01445 |
| Acyl-coA-binding protein, ACBP | (ZP_0095141) Croceibacter atlanticus |
| (EAT25091) Roseiflexus sp. RS-1 | HTCC2559 phosphatidylserine |
| Acyl-coA-binding protein, ACBP | decarboxylase |
| (XP_461327) Debaryomyces hansenii | (EAP5941) Croceibacter atlanticus |
| CBS767 | HTCC2559 phosphatidylserine |
| hypothetical protein DEHA0F24079g | decarboxylase |
| (AAT1164) Agave americana membrane | (AAZ10792) Trypanosoma brucei |
| acyl-CoA binding protein | acyl-CoA binding protein, putative |
| (ZP_01222732) Photobacterium profundum | (AAX7975) Trypanosoma brucei |
| 3TCK Hypothetical Acyl-CoA-binding | acyl-CoA binding protein, putative |
| protein | (CAA4461) acyl-CoA-binding protein/ |
| (EAS40711) Photobacterium profundum | diazepam-binding inhibitor [synthetic |
| 3TCK Hypothetical Acyl-CoA-binding | construct] |
| protein | (AAR1057) Oryza sativa (japonica cultivar- |
| (AAX5200) Aedes aegypti (yellow fever | group) |
| mosquito) | putative Acyl-CoA-binding protein |
| diazepam-binding inhibitor | (CAK1600) hypothetical protein |
| (2ABD) Bos taurus (cattle) | Pseudomonas |
| The Three-Dimensional Structure Of Acyl- | (CAJ03592) Leishmania major hypothetical |
| Coenzyme A Binding Protein From Bovine | protein, unknown function |
| Liver. Structural Refinement Using | (CAA69945) Saccharomyces monacensis |
| Heteronuclear Multidimensional Nmr | ORM1 |
| Spectroscopy | (CAA69943) Saccharomyces cerevisiae |
| (BAA97324) Arabidopsis thaliana (thale | (baker's yeast) |
| cress) | ORM1 |
| unnamed protein product | (NP_974227) Arabidopsis thaliana (thale |
| (AAM67425) Arabidopsis thaliana (thale | cress) acyl-CoA binding |
| cress) | (NP_17193) Arabidopsis thaliana (thale |
| AT4g2770/T27E11_20 | cress) |
| (AAL90917) Arabidopsis thaliana (thale | acyl-CoA binding |
| cress) | (NP_19115) Arabidopsis thaliana (thale |
| AT4g2770/T27E11_20 | cress) |
| (AAL5665) Mus musculus (house mouse) | acyl-CoA binding |
| diazepam binding inhibitor | (ZP_01252644) Psychroflexus torquis |
| (AAG46057) Arabidopsis thaliana (thale | ATCC 700755 hypothetical protein |
| cress) acyl-CoA binding protein 2 | P700755_02117 |
| (AAG46056) Arabidopsis thaliana (thale | (EAS72513) Psychroflexus torquis ATCC |
| cress) acyl-CoA binding protein ACBP2 | 700755 hypothetical protein |
| (1ACA) Bos taurus (cattle) Acyl-Coenzyme | P700755_02117 |
| A Binding Protein (Acbp) Complex With | (Q96495) Saccharomyces monacensis |
| Palmitoyl-Coenzyme A (Nmr, 20 | Protein ORM1 |
| Structures) | (AAF36031) Caenorhabditis elegans |
| (P53224) Saccharomyces cerevisiae | Hypothetical protein Y71H2B.1 |
| (baker's yeast) | (CAA9197) Caenorhabditis elegans |
| Protein ORM1 | Hypothetical protein F47B10.7 |
| (CAA6779) Caenorhabditis elegans | (CAG14939) Salmo salar (Atlantic salmon) |
| Hypothetical protein R06F6.9 | acyl-coenzyme A-binding protein |
| (YP_60799) Pseudomonas entomophila L4 | (AAB54171) Caenorhabditis elegans |
| hypothetical protein PSEEN3250 | Hypothetical protein C44E4.6 |
| (CAL53076) Ostreococcus tauri unnamed | (EAL21265) Cryptococcus neoformans var. |
| protein product | neoformans B-3501A |
| (EAX42942) Ralstonia pickettii 12J | hypothetical protein CNBD3190 |
| putative acyl-CoA-binding protein | (EAL21264) Cryptococcus neoformans var. |
| (CAB07319) Caenorhabditis elegans | neoformans B-3501A |
| Hypothetical protein C1D11.2 | hypothetical protein CNBD3190 |
| (AAK1960) Caenorhabditis elegans Acyl- | (EAL17562) Cryptococcus neoformans var. |
| coenzyme a binding protein protein 4 | neoformans B-3501A hypothetical protein |
| (P1624) Digitalis lanata Acyl-CoA-binding | CNBM120 |
| protein 1 | (AAL06793) Arabidopsis thaliana (thale |
| (CAB03343) Caenorhabditis elegans | cress) |
| Hypothetical protein T12D.3 | At1g3120/F5M6_26 |
| (CAA1944) Caenorhabditis elegans | (AAK55715) Arabidopsis thaliana (thale |
| Hypothetical protein Y17G7B.1 | cress) |
| (XP_0013151) Cryptosporidium parvum | At1g3120/F5M6_26 |
| lowa II conserved hypothetical protein | (NP_0010143) Danio rerio (zebrafish) |
| (AAI34171) Danio rerio (zebrafish) | hypothetical protein LOC553674 |
| Unknown (protein for MGC: 162964) | (NP_001034933) Homo sapiens (human) |
| (BAE91744) Macaca fascicularis (crab- | acyl-Coenzyme A binding domain |
| eating macaque) | containing 7 |
| unnamed protein product | (QBMP6) Mus musculus (house mouse) |
| (BAE9016) Macaca fascicularis (crab- | Golgi resident protein GCP60 (Acyl-CoA- |
| eating macaque) | binding domain-containing protein 3) (Golgi |
| unnamed protein product | phosphoprotein 1) (GOLPH1) (Golgi |
| (XP_001377556) Monodelphis domestica | complex-associated protein 1) (GOCAP1) |
| (gray short-tailed opossum) | (PBR- and PKA-associated protein 7) |
| PREDICTED: hypothetical protein | (Peripheral benzodiazepine receptor- |
| (XP_001367302) Monodelphis domestica | associated protein PAP7) |
| (gray short-tailed opossum) | (O75521) Homo sapiens (human) |
| PREDICTED: similar to Acyl-Coenzyme A | Peroxisomal 3,2-trans-enoyl-CoA |
| binding domain containing 5 isoform 2 | isomerase (Dodecenoyl-CoA isomerase) |
| (XP_001367254) Monodelphis domestica | (Delta(3),delta(2)-enoyl-CoA isomerase) |
| (gray short-tailed opossum) | (D3,D2-enoyl-CoA isomerase) (DBI-related |
| PREDICTED: similar to Acyl-Coenzyme A | protein 1) (DRS-1) (Hepatocellular |
| binding domain containing 5 isoform 1 | carcinoma-associated antigen) (Renal |
| (XP_001367923) Monodelphis domestica | carcinoma antigen NY-REN-1) |
| (gray short-tailed opossum) | (P56702) Rattus norvegicus (Norway rat) |
| PREDICTED: similar to Acyl-CoA-binding | Diazepam-binding inhibitor-like 5 |
| protein (ACBP) (Diazepam-binding | (Endozepine-like peptide) (ELP) |
| inhibitor) (DBI) (Endozepine) (EP) | (Q9WUR2) Mus musculus (house mouse) |
| (XP_0013602) Monodelphis domestica | Peroxisomal 3,2-trans-enoyl-CoA |
| (gray short-tailed opossum) | isomerase (Dodecenoyl-CoA isomerase) |
| PREDICTED: hypothetical protein | (Delta(3),delta(2)-enoyl-CoA isomerase) |
| (XP_001370361) Monodelphis domestica | (D3,D2-enoyl-CoA isomerase). |
| (gray short-tailed opossum) | (P07106) Bos taurus (cattle) Endozepine- |
| PREDICTED: similar to endozepine-like | related protein precursor (Membrane- |
| protein | associated |
| (XP_001375520) Monodelphis domestica | diazepam-binding inhibitor) (MA-DBI). |
| (gray short-tailed opossum) | (XP_00133919) Pichia stipitis CBS 6054 |
| PREDICTED: hypothetical protein | predicted protein |
| (XP_00137741) Monodelphis domestica | (YP_001022273) Methylibium |
| (gray short-tailed opossum) PREDICTED: | petroleiphilum PM1 putative acyl-CoA- |
| similar to Acyl-CoA-binding protein (ACBP) | binding protein |
| (Diazepam-binding inhibitor) (DBI) | (Q2KHT9) Bos taurus (cattle) Acyl-CoA- |
| (Endozepine) (EP) | binding domain-containing protein 4 |
| (XP_00136267) Monodelphis domestica | (Q9D061) Mus musculus (house mouse) |
| (gray short-tailed opossum) PREDICTED: | Acyl-CoA-binding domain-containing |
| hypothetical protein | protein 6 |
| (XP_001374542) Monodelphis domestica | (Q4VX4) Danio rerio (zebrafish) Acyl-CoA- |
| (gray short-tailed opossum) PREDICTED: | binding domain-containing protein 6. |
| similar to Acyl-Coenzyme A binding domain | (Q4V69) Xenopus laevis (African clawed |
| containing 6 | frog) |
| (XP_0013609) Pichia stipitis CBS 6054 | Acyl-CoA-binding domain-containing |
| predicted protein | protein 6 |
| Acyl-CoA-binding domain-containing | (Q66JD7) Xenopus tropicalis (Silurana |
| protein 7 | tropicalis) |
| (QNC06) Homo sapiens (human) | Acyl-CoA-binding domain-containing |
| Acyl-CoA-binding domain-containing | protein 6 |
| protein 4 | (QN6N7) Homo sapiens (human) |
| (NP_011551) Saccharomyces cerevisiae | (EAZ29205) Oryza sativa (japonica |
| (baker's yeast) | cultivar-group) hypothetical protein |
| Acb1p | OsJ_0126 |
| (EAZ6276) Pichia stipitis CBS 6054 | (EAZ27572) Oryza sativa (japonica |
| predicted protein | cultivar-group) |
| (NP_5734) Mus musculus (house mouse) | hypothetical protein OsJ_011055 |
| acyl-Coenzyme A binding domain | (EAZ2623) Oryza sativa (japonica cultivar- |
| containing 3 | group) hypothetical protein OsJ_009721 |
| (ABN6590) Pichia stipitis CBS 6054 | (EAZ05693) Oryza sativa (indica cultivar- |
| predicted protein | group) |
| (XP_00135651) Drosophila pseudoobscura | hypothetical protein OsI_026925 |
| GA21120-PA | (EAY99409) Oryza sativa (indica cultivar- |
| (XP_001356156) Drosophila | group) |
| pseudoobscura GA21340-PA | hypothetical protein OsI_020642 |
| (XP_001355732) Drosophila | (EAY9607) Oryza sativa (indica cultivar- |
| pseudoobscura GA17261-PA | group) |
| (XP_001354572) Drosophila | hypothetical protein OsI_017320 |
| pseudoobscura GA1245-PA | (EAY92477) Oryza sativa (indica cultivar- |
| (XP_00135333) Drosophila pseudoobscura | group) |
| GA21220-PA | hypothetical protein OsI_013710 |
| (XP_001353337) Drosophila | (EAY90744) Oryza sativa (indica cultivar- |
| pseudoobscura GA13977-PA | group) |
| (XP_001353336) Drosophila | hypothetical protein OsI_011977 |
| pseudoobscura GA2121-PA | (EAY90741) Oryza sativa (indica cultivar- |
| (XP_001353090) Drosophila | group) |
| pseudoobscura GA19142-PA | hypothetical protein OsI_011974 |
| (XP_001331712) Danio rerio (zebrafish) | (EAY9220) Oryza sativa (indica cultivar- |
| PREDICTED: hypothetical protein | group) |
| (XP_69059) Danio rerio (zebrafish) | hypothetical protein OsI_010453 |
| PREDICTED: hypothetical protein | (NP_99924) Sus scrofa (pig) |
| (XP_00133532) Danio rerio (zebrafish) | diazepam binding inhibitor |
| PREDICTED: similar to Acbd7 protein | (NP_00264) Mus musculus (house mouse) |
| (ABM9603) Methylibium petroleiphilum | acyl-Coenzyme A binding domain |
| PM1 putative acyl-CoA-binding protein | containing 4 isoform 1 |
| (AAH4371) Mus musculus (house mouse) | (EAY6523) Burkholderia dolosa AUO15 |
| Acyl-Coenzyme A binding domain | Hypothetical Acyl-CoA-binding protein |
| containing 4 | (XP_001349426) Plasmodium falciparum |
| (EAZ41613) Oryza sativa (japonica | 3D7 |
| cultivar-group) hypothetical protein | hypothetical protein, conserved |
| OsJ_025096 | (CAK94304) Paramecium tetraurelia |
| (EAZ35610) Oryza sativa (japonica | unnamed protein product |
| cultivar-group) | (CAK91204) Paramecium tetraurelia |
| hypothetical protein OsJ_019093 | unnamed protein product |
| (EAZ32450) Oryza sativa (japonica | (CAK69311) Paramecium tetraurelia |
| cultivar-group)hypothetical protein | unnamed protein product |
| OsJ_015933 | (CAK63532) Paramecium tetraurelia |
| Acyl-coA-binding protein, ACBP; | unnamed protein product |
| Serine/threonine protein | (ABN0040) Medicago truncatula (barrel |
| phosphatase, BSU1 | medic) |
| (NP_067269) Mus musculus (house | Diazepam-binding inhibitor-like 5 |
| mouse) | (Endozepine-like peptide) (ELP). |
| diazepam binding inhibitor-like 5 | (NP_99162) Danio rerio (zebrafish) |
| (CAL56467) Ostreococcus tauri | acyl-Coenzyme A binding domain |
| membrane acyl-CoA binding protein (ISS) | containing 3 |
| (CAL5444) Ostreococcus tauri Acyl-CoA- | (CAI42127) Homo sapiens (human) |
| binding protein (ISS) | peroxisomal D3,D2-enoyl-CoA isomerase |
| (CAL54274) Ostreococcus tauri Host cell | (CAI42125) Homo sapiens (human) |
| transcription factor HCFC1 (ISS) | peroxisomal D3,D2-enoyl-CoA isomerase |
| (Q5RJK) Rattus norvegicus (Norway rat) | (CAI35101) Mus musculus (house mouse) |
| Acyl-CoA-binding domain-containing | diazepam binding inhibitor-like 5 |
| protein 6 | (CAI21107) Danio rerio (zebrafish) |
| (Q9BR61) Homo sapiens (human) | novel protein (zgc: 66303) |
| Acyl-CoA-binding domain-containing | (CAI21106) Danio rerio (zebrafish) novel |
| protein 6 | protein (zgc: 66303) |
| (Q6DGF9) Rattus norvegicus (Norway rat) | (CAI19365) Homo sapiens (human) acyl- |
| Acyl-CoA-binding domain-containing | Coenzyme A binding domain containing 6 |
| protein 4 | (CAI15093) Homo sapiens (human) acyl- |
| (AAI1412) Bos taurus (cattle) Hypothetical | Coenzyme A binding domain containing 6 |
| LOC76330 | (CAI16916) Homo sapiens (human) acyl- |
| (AAI126) Bos taurus (cattle) | Coenzyme A binding domain containing 5 |
| Similar to acyl-Coenzyme A binding | (CAI16915) Homo sapiens (human) |
| domain containing 4 | acyl-Coenzyme A binding domain |
| (AAI03432) Bos taurus (cattle) | containing 5 |
| DBIL5 protein | (CAI16914) Homo sapiens (human) acyl- |
| (AAI02900) Bos taurus (cattle) | Coenzyme A binding domain containing 5 |
| Similar to Acyl-CoA-binding protein (ACBP) | (CAI16913) Homo sapiens (human) acyl- |
| (Diazepam binding inhibitor) (DBI) | Coenzyme A binding domain containing 5 |
| (Endozepine) (EP) | (CAI16912) Homo sapiens (human) |
| (AAI02374) Bos taurus (cattle) | acyl-Coenzyme A binding domain |
| AAI02374 | containing 5 |
| (AAI02907) Bos taurus (cattle) Peroxisomal | (CAH71922) Homo sapiens (human) acyl- |
| D3,D2-enoyl-CoA isomerase | Coenzyme A binding domain containing 3 |
| (Q9H3P7) Homo sapiens (human) | (CAH73964) Homo sapiens (human) novel |
| Golgi resident protein GCP60 (Acyl-CoA- | protein (FLJ3219) |
| binding domain-containing protein 3) (Golgi | (CAH71747) Homo sapiens (human) |
| phosphoprotein 1) (GOLPH1) (Golgi | acyl-Coenzyme A binding domain |
| complex-associated protein 1) (GOCAP1) | containing 6 |
| (PBR- and PKA-associated protein 7) | (2FJ9_A) Chain A, High Resolution Crystal |
| (Peripheral benzodiazepine receptor- | Structure Of The Unliganded Human Acbp. |
| associated protein PAP7) | (2CB_B) Homo sapiens (human) Chain B, |
| (Q7TNY6) Rattus norvegicus (Norway rat) | High Resolution Crystal Structure Of |
| Golgi resident protein GCP60 (Acyl-CoA- | Liganded Human L-Acbp |
| binding domain-containing protein 3) (Golgi | (2CB_A) Homo sapiens (human) Chain A, |
| phosphoprotein 1) (GOLPH1) (Golgi | High Resolution Crystal Structure Of |
| complex-associated protein 1) (GOCAP1) | Liganded Human L-Acbp |
| (DMT1-associated protein) (DAP) | (XP_73333) Plasmodium chabaudi |
| (O09035) Mus musculus (house mouse) | chabaudi |
| Diazepam binding inhibitor (GABA receptor | hypothetical protein PC30252.00.0 |
| modulator, acyl-Coenzyme A binding | (AAH62996) Homo sapiens (human) |
| protein) | (EAW6059) Homo sapiens (human) acyl- |
| (NP_93216) Ashbya gossypii ATCC 1095 | Coenzyme A binding domain containing 5, |
| (Eremothecium gossypii ATCC 1095) | isoform CRA_a |
| ACL1Wp | (EAW77509) Homo sapiens (human) |
| (NP_92677) Ashbya gossypii ATCC 1095 | hCG165200 |
| (Eremothecium gossypii ATCC 1095) | (EAW69776) Homo sapiens (human) |
| AAR135Wp | acyl-Coenzyme A binding domain |
| (XP_55041) Bos taurus (cattle) | containing 3, isoform CRA_a |
| PREDICTED: hypothetical protein | (EAW69775) Homo sapiens (human) |
| (AAH97225) Danio rerio (zebrafish) Acbd7 | acyl-Coenzyme A binding domain |
| protein | containing 3, isoform CRA_a |
| (YP_53546) Ralstonia metallidurans CH34 | (EAW55163) Homo sapiens (human) |
| acyl-coA-binding protein, ACBP | peroxisomal D3,D2-enoyl-CoA isomerase, |
| (NP_001029414) Bos taurus (cattle) | isoform CRA_e |
| peroxisomal D3,D2-enoyl-CoA isomerase | (EAW55162) Homo sapiens (human) |
| (YP_296149) Ralstonia eutropha JMP134 | peroxisomal D3,D2-enoyl-CoA isomerase, |
| Acyl-coA-binding protein, ACBP | isoform CRA_a |
| (EAW95216) Homo sapiens (human) | (EAW55160) Homo sapiens (human) |
| diazepam binding inhibitor (GABA receptor | peroxisomal D3,D2-enoyl-CoA isomerase, |
| modulator, acyl-Coenzyme A binding | isoform CRA_c |
| protein), isoform CRA_a | (EAW5515) Homo sapiens (human) |
| (EAW95215) Homo sapiens (human) | peroxisomal D3,D2-enoyl-CoA isomerase, |
| diazepam binding inhibitor (GABA receptor | isoform CRA_a |
| modulator, acyl-Coenzyme A binding | (NP_001020626) Danio rerio |
| protein), isoform CRA_a | (zebrafish)acyl-Coenzyme A binding |
| (EAW95214) Homo sapiens (human) | domain containing 6 |
| diazepam binding inhibitor (GABA receptor | (XP_001237266) Anopheles gambiae str. |
| modulator, acyl-Coenzyme A binding | PEST |
| protein), isoform CRA_a | ENSANGP0000003176 |
| (EAW9104) Homo sapiens (human) | (XP_31127) Anopheles gambiae str. PEST |
| acyl-Coenzyme A binding domain | ENSANGP00000017422 |
| containing 6, isoform CRA_a | (XP_31377) Anopheles gambiae str. PEST |
| (EAW9103) Homo sapiens (human) acyl- | ENSANGP0000001167 |
| Coenzyme A binding domain containing 6, | (XP_3123) Anopheles gambiae str. PEST |
| isoform CRA_a | ENSANGP00000014744 |
| (EAW9102) Homo sapiens (human) | (XP_30405) Anopheles gambiae str. PEST |
| acyl-Coenzyme A binding domain | ENSANGP00000019171 |
| containing 6, isoform CRA_a | (AAS51040) Ashbya gossypii ATCC 1095 |
| (EAW90652) Homo sapiens (human) | (Eremothecium gossypii ATCC 1095) |
| hCG1646635, isoform CRA_b | ACL1Wp |
| (EAW6243) Homo sapiens (human) | (AAS50501) Ashbya gossypii ATCC 1095 |
| hCG2017592 | (Eremothecium gossypii ATCC 1095) |
| (EAW6062) Homo sapiens (human) | AAR135Wp |
| acyl-Coenzyme A binding domain | (NP_00107222) Xenopus tropicalis |
| containing 5, isoform CRA_d | (Silurana tropicalis) |
| (EAW6060) Homo sapiens (human) | hypothetical protein LOC7023 |
| acyl-Coenzyme A binding domain | (AAI21677) Xenopus tropicalis (Silurana |
| containing 5, isoform CRA_b | tropicalis) |
| (YP_99060) Francisella tularensis subsp. | Hypothetical protein MGC147507 |
| novicida U112 bifunctional protein: 3- | (AAI131) Xenopus tropicalis (Silurana |
| hydroxacyl-CoA dehydrogenase/acyl-CoA- | tropicalis) MGC146543 protein |
| binding protein | (XP_41965) Gallus gallus (red jungle fowl) |
| (Q3SZF0) Bos taurus (cattle) | PREDICTED: hypothetical protein isoform 2 |
| Acyl-CoA-binding domain-containing | (NP_001002645) Danio rerio (zebrafish) |
| protein 7 | hypothetical protein LOC43691 |
| (Q5R7P6) Pongo pygmaeus (orangutan) | (CAG7424) Debaryomyces hansenii |
| Acyl-CoA-binding domain-containing | CBS767 |
| protein 4 | unnamed protein product |
| (Q9MZG3) Bos taurus (cattle) | (CAG7727) Yarrowia lipolytica CLIB122 |
| Diazepam-binding inhibitor-like 5 | unnamed protein product |
| (Endozepine-like peptide) (ELP). | (CAH0210) Kluyveromyces lactis NRRL Y- |
| (XP_00123129) Gallus gallus (red jungle | 1140 |
| fowl) | unnamed protein product |
| PREDICTED: hypothetical protein isoform 1 | (CAH02670) Kluyveromyces lactis NRRL |
| (XP_429769) Gallus gallus (red jungle fowl) | Y-1140 unnamed protein product |
| PREDICTED: similar to ACBP/DBI | (CAG61365) Candida glabrata CBS 13 |
| (NP_001071103) Rattus norvegicus | unnamed protein product |
| (Norway rat) | (NP_99260) Danio rerio (zebrafish) |
| acyl-Coenzyme A binding domain | acyl-Coenzyme A binding domain |
| containing 5 | containing 4 |
| (NP_001039679) Bos taurus (cattle) | (CAG20610) Photobacterium profundum |
| hypothetical protein LOC5154 | SS9 Hypothetical Acyl-CoA-binding protein |
| (NP_00103593) Homo sapiens (human) | (NP_99907) Gallus gallus (chicken) |
| NP_00103593 | diazepam binding inhibitor (GABA receptor |
| (NP_00100065) Xenopus tropicalis | modulator, acyl-Coenzyme A binding |
| (Silurana tropicalis) MGC79661 protein | protein) |
| (NP_001026214) Gallus gallus (chicken) | (NP_00610) Homo sapiens (human) |
| acyl-Coenzyme A binding domain | peroxisomal D3,D2-enoyl-CoA isomerase |
| containing 3 | isoform 1 |
| (NP_001012013) Rattus norvegicus | (NP_974) Xenopus tropicalis (Silurana |
| (Norway rat) | tropicalis) diazepam binding inhibitor |
| NP_001012013 | (GABA receptor modulator, acyl-Coenzyme |
| (NP_001011906) Rattus norvegicus | A binding protein |
| (Norway rat) NP_001011906 | (NP_7263) Rattus norvegicus (Norway rat) |
| (NP_001006356) Gallus gallus (chicken) | DMT1-associated protein |
| acyl-Coenzyme A binding domain | (NP_5131) Bos taurus (cattle) |
| containing 5 | diazepam binding inhibitor |
| (NP_0010010) Xenopus tropicalis (Silurana | (NP_4792) Bos taurus (cattle) |
| tropicalis) | diazepam binding inhibitor-like 5 |
| acbd5 protein | (NP_03069) Mus musculus (house mouse) |
| (NP_955902) Danio rerio (zebrafish) | acyl-Coenzyme A binding domain |
| diazepam binding inhibitor | containing 5 |
| (NP_001006967) Rattus norvegicus | (NP_663736) Homo sapiens (human) acyl- |
| (Norway rat) | Coenzyme A binding domain containing 5 |
| peroxisomal delta3, delta2-enoyl- | (NP_02526) Mus musculus (house |
| Coenzyme A isomerase | mouse)acyl-Coenzyme A binding domain |
| (NP_51947) Ralstonia solanacearum | containing 6 |
| GMI1000 PROBABLE ACYL-COA- | (NP_01223) Mus musculus (house mouse) |
| BINDING PROTEIN | NP_01223 |
| (CAD1506) Ralstonia solanacearum | (EAU4611) Coprinopsis cinerea |
| probable acyl-coa-binding protein | okayama7#130 (Coprinus cinereus |
| (NP_073572) Homo sapiens (human) acyl- | okayama7#130) |
| Coenzyme A binding domain containing 3 | predicted protein |
| (NP_115736) Homo sapiens (human) | (XP_00122149) Chaetomium globosum |
| acyl-Coenzyme A binding domain | CBS 14.51 |
| containing 6 | hypothetical protein CHGG_10222 |
| (NP_0799) Homo sapiens (human) | (XP_001223100) Chaetomium globosum |
| acyl-Coenzyme A binding domain | CBS 14.51 |
| containing 4 | hypothetical protein CHGG_036 |
| (NP_067607) Rattus norvegicus (Norway | (EAU77246) Anopheles gambiae str. PEST |
| rat) endozepine-like peptide | ENSANGP0000003176 |
| (NP_03599) Mus musculus (house mouse) | (EAA0661) Anopheles gambiae str. PEST |
| peroxisomal delta3, delta2-enoyl- | ENSANGP00000017422 |
| Coenzyme A isomerase | (NP_00102706) Drosophila melanogaster |
| (CAA7994) Rattus norvegicus (Norway rat) | (fruit fly) |
| diazepam binding inhibitor | CG33713-PA, isoform A |
| (CAA5326) Drosophila melanogaster (fruit | (NP_00102705) Drosophila melanogaster |
| fly) | (fruit fly) |
| diazepam binding | CG33713-PB, isoform B |
| inhibitor/endozepine/acyl-CoA-binding | (CAJ09636) Leishmania major hypothetical |
| homologue | protein, conserved |
| (AAI06605) Xenopus laevis (African clawed | (CAJ03603) Leishmania major |
| frog) | hypothetical protein, conserved |
| AAI06605 | (CAJ03596) Leishmania major hypothetical |
| (AAH99293) Xenopus laevis (African | protein, conserved |
| clawed frog) | (EAA04566) Anopheles gambiae str. PEST |
| MGC11645 protein | ENSANGP00000019171 |
| (AAH97519) Xenopus laevis (African | (EAA034) Anopheles gambiae str. PEST |
| clawed frog) MGC114637 protein | ENSANGP00000014744 |
| (ABK27612) Rattus norvegicus (Norway | (EAA09122) Anopheles gambiae str. PEST |
| rat) | ENSANGP0000001167 |
| acyl-CoA binding domain protein | (NP_523952) Drosophila melanogaster |
| (ABK27611) Rattus norvegicus (Norway | (fruit fly) |
| rat) acyl-CoA binding domain protein | Diazepam-binding inhibitor CG627-PB, |
| (XP_46297) Trypanosoma brucei | isoform B |
| TREU927 | (NP_72921) Drosophila melanogaster (fruit |
| hypothetical protein, conserved | fly) |
| (EAU93205) Coprinopsis cinerea | Diazepam-binding inhibitor CG627-PA, |
| okayama7#130 (Coprinus cinereus | isoform A |
| okayama7#130) | (NP_6403) Drosophila melanogaster (fruit |
| predicted protein | fly) CG62-PA |
| (EAU5363) Coprinopsis cinerea | (NP_6402) Drosophila melanogaster (fruit |
| okayama7#130 (Coprinus cinereus | fly) |
| okayama7#130) | CG1529-PA |
| predicted protein | (NP_60917) Drosophila melanogaster (fruit |
| CG629-PA | fly) |
| (NP_64255) Drosophila melanogaster (fruit | CG49-PA |
| fly) CG504-PA | (NP_6401) Drosophila melanogaster (fruit |
| (NP_60729) Drosophila melanogaster (fruit | fly) |
| fly) | unnamed protein product |
| CG14-PA | (BAE3934) Mus musculus (house mouse) |
| (NP_6034) Drosophila melanogaster (fruit | unnamed protein product |
| fly) | (BAE2174) Mus musculus (house mouse) |
| CG14232-PA | unnamed protein product |
| (XP_0011222) Strongylocentrotus | (BAE3404) Mus musculus (house mouse) |
| purpuratus | unnamed protein product |
| PREDICTED: hypothetical protein | (NP_49917) Caenorhabditis elegans |
| (XP_001177795) Strongylocentrotus | T12D.3 |
| purpuratus PREDICTED: similar to | (NP_50922) Caenorhabditis elegans Acyl- |
| GA21120-PA | Coenzyme A Binding Protein family |
| (XP_73927) Strongylocentrotus purpuratus | member (acbp-3) |
| PREDICTED: similar to MGC79661 | (BAC34262) Mus musculus (house mouse) |
| protein, partial | unnamed protein product |
| (XP_74299) Strongylocentrotus purpuratus | (NP_49609) Caenorhabditis elegans |
| PREDICTED: similar to GA21120-PA | Acyl-Coenzyme A Binding Protein family |
| (XP_70031) Strongylocentrotus purpuratus | member (acbp-4) |
| PREDICTED: hypothetical protein isoform 1 | (NP_499531) Caenorhabditis elegans |
| (NP_001041025) Caenorhabditis elegans | Membrane Associated Acyl-CoA binding |
| Y41E3.7a | protein family member (maa-1) |
| (ABG9952) Rhodococcus sp. RHA1 | (NP_496552) Caenorhabditis elegans |
| possible acyl-CoA-binding protein | Y17G7B.1 |
| (ABG96) Rhodococcus sp. RHA1 probable | (NP_496330) Caenorhabditis elegans |
| acyl-CoA-binding protein | Enoyl-CoA Hydratase family member (ech- |
| (AAI1551) Mus musculus (house mouse) | 4) |
| Acyl-Coenzyme A binding domain | (NP_491412) Caenorhabditis elegans Acyl- |
| containing 6 | Coenzyme A Binding Protein family |
| (AAI1552) Mus musculus (house mouse) | member (acbp-1) |
| Acyl-Coenzyme A binding domain | (XP_0012037) Aspergillus terreus NIH2624 |
| containing 6 | predicted protein |
| (CAJ1905) Xenopus tropicalis (Silurana | (BAC2565) Mus musculus (house mouse) |
| tropicalis) | unnamed protein product |
| acyl-Coenzyme A binding domain | (BAB26315) Mus musculus (house mouse) |
| containing 6 | unnamed protein product |
| (CAJ2921) Xenopus tropicalis (Silurana | (BAB23735) Mus musculus (house mouse) |
| tropicalis) | unnamed protein product |
| diazepam binding inhibitor (dbi) | (BAC2692) Mus musculus (house mouse) |
| (NP_001033359) Caenorhabditis elegans | unnamed protein product |
| F26A1.15 | (BAB32175) Mus musculus (house mouse) |
| (BAE2656) Mus musculus (house mouse) | unnamed protein product |
| unnamed protein product | (BAB31366) Mus musculus (house mouse) |
| (BAE42023) Mus musculus (house mouse) | unnamed protein product |
| unnamed protein product | (BAB25755) Mus musculus (house mouse) |
| (BAE26340) Mus musculus (house mouse) | unnamed protein product |
| unnamed protein product | (BAB25730) Mus musculus (house mouse) |
| (BAE20705) Mus musculus (house mouse) | unnamed protein product |
| fusion product of 3-hydroxacyl-CoA | (BAB24637) Mus musculus (house mouse) |
| dehydrogenase and acyl-CoA-binding | unnamed protein product |
| protein | (CAJ79024) Francisella tularensis subsp. |
| (YP_76316) Francisella tularensis subsp. | holarctica LVS |
| holarctica OSU1 3-hydroxyacyl-CoA | PREDICTED: similar to diazepam-binding |
| dehydrogenase | protein isoform 2 |
| (ABI2549) Francisella tularensis subsp. | (XP_001156427) Pan troglodytes |
| holarctica OSU1 3-hydroxyacyl-CoA | (chimpanzee) |
| dehydrogenase | PREDICTED: similar to diazepam-binding |
| (XP_523669) Pan troglodytes | protein isoform 1 |
| (chimpanzee) | (XP_515759) Pan troglodytes |
| PREDICTED: hypothetical protein isoform 6 | (chimpanzee) |
| (XP_001142506) Pan troglodytes | PREDICTED: diazepam binding inhibitor |
| (chimpanzee) | isoform 3 |
| PREDICTED: hypothetical protein isoform 4 | (XP_00114055) Pan troglodytes |
| (XP_001142437) Pan troglodytes | (chimpanzee) |
| (chimpanzee) | PREDICTED: hypothetical protein isoform 1 |
| PREDICTED: hypothetical protein isoform 3 | (XP_00114064) Pan troglodytes |
| (XP_001142352) Pan troglodytes | (chimpanzee) |
| (chimpanzee) | PREDICTED: hypothetical protein isoform 2 |
| PREDICTED: hypothetical protein isoform 2 | (XP_52495) Pan troglodytes (chimpanzee) |
| (XP_001142572) Pan troglodytes | PREDICTED: acyl-Coenzyme A binding |
| (chimpanzee) | domain containing 6 |
| PREDICTED: acyl-Coenzyme A binding | (BAC11403) Homo sapiens (human) |
| domain containing 4 isoform 5 | unnamed protein product |
| (XP_001142266) Pan troglodytes | (BAB15159) Homo sapiens (human) |
| (chimpanzee) | unnamed protein product |
| PREDICTED: acyl-Coenzyme A binding | (BAB14553) Homo sapiens (human) |
| domain containing 4 isoform 1 | unnamed protein product |
| (XP_001171767) Pan troglodytes | (EAU3229) Aspergillus terreus NIH2624 |
| (chimpanzee) | predicted protein |
| PREDICTED: hypothetical protein | (XP_945054) Homo sapiens (human) |
| (XP_507712) Pan troglodytes | PREDICTED: similar to Acyl-CoA-binding |
| (chimpanzee) PREDICTED: acyl- | protein (ACBP) (Diazepam-binding |
| Coenzyme A binding domain containing 5 | inhibitor) (DBI) (Endozepine) (EP) |
| (XP_001140464) Pan troglodytes | (XP_933945) Homo sapiens (human) |
| (chimpanzee) | PREDICTED: similar to Acyl-CoA-binding |
| PREDICTED: acyl-Coenzyme A binding | protein (ACBP) (Diazepam-binding |
| domain containing 7 | inhibitor) (DBI) (Endozepine) |
| (XP_001162544) Pan troglodytes | (YP_559354) Burkholderia xenovorans |
| (chimpanzee) | LB400 |
| PREDICTED: hypothetical protein isoform 4 | Putative acyl-CoA-binding protein |
| (XP_527221) Pan troglodytes | (YP_707740) Rhodococcus sp. RHA1 |
| (chimpanzee) PREDICTED: hypothetical | possible acyl-CoA-binding protein |
| protein isoform 5 | (YP_707026) Rhodococcus sp. RHA1 |
| (XP_00115640) Pan troglodytes | probable acyl-CoA-binding protein |
| (chimpanzee) | (AAN12074) Drosophila melanogaster (fruit |
| (AAF5034) Drosophila melanogaster (fruit | fly) |
| fly) | CG627-PB, isoform B |
| CG33713-PA, isoform A | (AAN09671) Drosophila melanogaster (fruit |
| (AAF5060) Drosophila melanogaster (fruit | fly) |
| fly) | CG33713-PB, isoform B |
| CG62-PA | (BAF0000) Arabidopsis thaliana (thale |
| (AAF52610) Drosophila melanogaster (fruit | cress) |
| fly) CG49-PA | hypothetical protein |
| (AAF5115) Drosophila melanogaster (fruit | (XP_394773) Apis mellifera (honey bee) |
| fly) | PREDICTED: similar to CG14232-PA |
| CG14-PA | (XP_394745) Apis mellifera (honey bee) |
| (AAF50610) Drosophila melanogaster (fruit | PREDICTED: similar to CG49-PA |
| fly) | (AAH9715) Danio rerio (zebrafish) |
| CG629-PA | Acyl-Coenzyme A binding domain |
| (AAF50609) Drosophila melanogaster (fruit | containing 6 |
| fly) | (AAH95655) Danio rerio (zebrafish) |
| CG1529-PA | Zgc: 112043 |
| (AAF50607) Drosophila melanogaster (fruit | (AAH45533) Homo sapiens (human) |
| fly) | Acyl-Coenzyme A binding domain |
| CG627-PA, isoform A | containing 3 |
| (AAF50367) Drosophila melanogaster (fruit | (AAH54676) Danio rerio (zebrafish) |
| fly) | Acyl-Coenzyme A binding domain |
| CG504-PA | containing 3 |
| (AAF49009) Drosophila melanogaster (fruit | (AAH659) Rattus norvegicus (Norway rat) |
| fly) | Acyl-Coenzyme A binding domain |
| CG14232-PA | containing 6 |
| (XP_63550) Dictyostelium discoideum AX4 | (AAH3341) Homo sapiens (human) |
| hypothetical protein DDBDRAFT_01797 | Peroxisomal D3,D2-enoyl-CoA isomerase |
| (AAH5351) Mus musculus (house mouse) | (AAH4717) Rattus norvegicus (Norway rat) |
| Acbd5 protein | Diazepam binding inhibitor |
| (AAH35202) Mus musculus (house mouse) | (AAH1671) Homo sapiens (human) |
| Acbd5 protein | Peroxisomal D3,D2-enoyl-CoA isomerase |
| (BAD363) Homo sapiens (human) putative | (AAH377) Rattus norvegicus (Norway rat) |
| protein product of HMFT0700 | Acyl-Coenzyme A binding domain |
| (AAH14724) Mus musculus (house mouse) | containing 3 |
| RIKEN cDNA 110022C23 gene | (AAH3764) Rattus norvegicus (Norway rat) |
| (EAT661) Phaeosphaeria nodorum SN15 | Peroxisomal delta3, delta2-enoyl- |
| hypothetical protein SNOG_05554 | Coenzyme A isomerase |
| (EAT79079) Phaeosphaeria nodorum | (AAH17474) Homo sapiens (human) |
| SN15 predicted protein | Peroxisomal D3,D2-enoyl-CoA isomerase |
| (XP_001122639) Apis mellifera (honey | (AAH0266) Homo sapiens (human) |
| bee) | Peroxisomal D3,D2-enoyl-CoA isomerase |
| PREDICTED: similar to CG14-PA | (AAH2499) Xenopus tropicalis (Silurana |
| (XP_00112314) Apis mellifera (honey bee) | tropicalis) |
| PREDICTED: similar to CG33713-PB, | Acbd5 protein |
| isoform B | (AAH0953) Xenopus tropicalis (Silurana |
| (BAE99122) Arabidopsis thaliana (thale | tropicalis) |
| cress) | MGC79661 protein |
| hypothetical protein | (AAH76531) Danio rerio (zebrafish) |
| (AAH6326) Danio rerio (zebrafish) | Zgc: 92030 |
| Zgc: 5611 | (AAH7637) Rattus norvegicus (Norway rat) |
| (AAH6245) Danio rerio (zebrafish) | Acyl-Coenzyme A binding domain |
| AAH6245 | containing 4 |
| (AAH60602) Mus musculus (house mouse) | PREDICTED: similar to Acyl-CoA-binding |
| Acyl-Coenzyme A binding domain | protein (ACBP) (Diazepam-binding |
| containing 3 | inhibitor) (DBI) (Endozepine) (EP) |
| (AAH59746) Xenopus tropicalis (Silurana | (XP_0010009) Rattus norvegicus (Norway |
| tropicalis) | rat) |
| Diazepam binding inhibitor (dbi) | PREDICTED: similar to Acyl-CoA-binding |
| (AAH4474) Mus musculus (house mouse) | protein (ACBP) (Diazepam-binding |
| Diazepam binding inhibitor-like 5 | inhibitor) (DBI) (Endozepine) (EP) |
| (AAH45916) Danio rerio (zebrafish) | (XP_577252) Rattus norvegicus (Norway |
| Zgc: 77734 | rat) |
| (AAH274) Mus musculus (house mouse) | PREDICTED: similar to Acyl-CoA-binding |
| Diazepam binding inhibitor | protein (ACBP) (Diazepam-binding |
| (AAH06505) Homo sapiens (human) | inhibitor) (DBI) (Endozepine) (EP) |
| Acyl-Coenzyme A binding domain | (XP_001062202) Rattus norvegicus |
| containing 6 | (Norway rat) |
| (ABF99749) Oryza sativa (japonica | PREDICTED: similar to acyl-Coenzyme A |
| cultivar-group) | binding domain containing 5 |
| acyl-CoA binding family protein, putative, | (XP_001053520) Rattus norvegicus |
| expressed | (Norway rat) |
| (ABF9974) Oryza sativa (japonica cultivar- | PREDICTED: similar to acyl-Coenzyme A |
| group) | binding domain containing 5 |
| acyl-CoA binding family protein, putative, | (AAW27239) Schistosoma japonicum |
| expressed | SJCHGC040 protein |
| (ABF99747) Oryza sativa (japonica | (XP_00111439) Macaca mulatta (rhesus |
| cultivar-group) | monkey) |
| acyl-CoA binding family protein, putative, | PREDICTED: similar to diazepam binding |
| expressed | inhibitor, partial |
| (ABF97253) Oryza sativa (japonica | (XP_001115247) Macaca mulatta (rhesus |
| cultivar-group) | monkey) |
| Acyl-CoA-binding protein, putative | PREDICTED: similar to acyl-Coenzyme A |
| (AAH30555) Homo sapiens (human) | binding domain containing 4 |
| Acyl-Coenzyme A binding domain | (XP_001117210) Macaca mulatta (rhesus |
| containing 5 | monkey) |
| (XP_001072124) Rattus norvegicus | PREDICTED: similar to diazepam binding |
| (Norway rat) | inhibitor-like 5 |
| PREDICTED: similar to Acyl-CoA-binding | (XP_00107153) Macaca mulatta (rhesus |
| protein (ACBP) (Diazepam-binding | monkey) |
| inhibitor) (DBI) (Endozepine) (EP) | PREDICTED: similar to diazepam binding |
| (XP_341563) Rattus norvegicus (Norway | inhibitor |
| rat) | (XP_001103096) Macaca mulatta (rhesus |
| PREDICTED: similar to Acyl-CoA-binding | monkey) |
| protein (ACBP) (Diazepam-binding | PREDICTED: diazepam binding inhibitor |
| inhibitor) (DBI) (Endozepine) (EP) | (GABA receptor modulator, acyl-Coenzyme |
| (XP_001054001) Rattus norvegicus | A binding protein) |
| (Norway rat) | (XP_001091346) Macaca mulatta (rhesus |
| PREDICTED: similar to peroxisomal | monkey) |
| D3,D2-enoyl-CoA isomerase isoform 1 | PREDICTED: similar to Acyl-CoA-binding |
| (XP_001115044) Macaca mulatta (rhesus | protein (ACBP) (Diazepam-binding |
| monkey) | inhibitor) (DBI) (Endozepine) (EP) |
| PREDICTED: similar to acyl-Coenzyme A | (XP_001094776) Macaca mulatta (rhesus |
| binding domain containing 6 | monkey) |
| (XP_001091471) Macaca mulatta (rhesus | (XP_36927) Magnaporthe grisea 70-15 |
| monkey) | hypothetical protein MG06177.4 |
| PREDICTED: similar to acyl-Coenzyme A | (XP_360613) Magnaporthe grisea 70-15 |
| binding domain containing 3 | hypothetical protein MG03156.4 |
| (EAT42240) Aedes aegypti (Stegomyia | (XP_459250) Debaryomyces hansenii |
| egypti) | CBS767 |
| conserved hypothetical protein | hypothetical protein DEHA0D1905g |
| (EAT4211) Aedes aegypti (Stegomyia | (XP_96230) Neurospora crassa OR74A |
| egypti) | hypothetical protein |
| conserved hypothetical protein | (XP_95716) Neurospora crassa OR74A |
| (EAT42117) Aedes aegypti (Stegomyia | hypothetical protein (endozepine related |
| egypti) | protein) |
| conserved hypothetical protein | (XP_995791) Mus musculus (house |
| (EAT39927) Aedes aegypti (Stegomyia | mouse) |
| egypti) | PREDICTED: similar to Acyl-CoA-binding |
| V-1 protein, putative | protein (ACBP) (Diazepam-binding |
| (EAT3939) Aedes aegypti (Stegomyia | inhibitor) (DBI) (Endozepine) (EP) |
| egypti) | (ABF0277) Ralstonia metallidurans CH34 |
| diazepam binding inhibitor, putative | acyl-coA-binding protein, ACBP |
| (EAT33) Aedes aegypti (Stegomyia | (XP_9131) Mus musculus (house mouse) |
| egypti) | PREDICTED: similar to Acyl-CoA-binding |
| diazepam binding inhibitor, putative | protein (ACBP) (Diazepam-binding |
| (AAH29526) Homo sapiens (human) | inhibitor) (DBI) (Endozepine) (EP) |
| ACBD7 protein | (XP_44966) Mus musculus (house mouse) |
| (AAY42394) Lyngbya majuscula | PREDICTED: hypothetical protein |
| HctB | LOC7245 |
| (ZP_01347067) Burkholderia mallei 10399 | (XP_2434) Trypanosoma brucei TREU927 |
| hypothetical protein Bmal10_03000377 | hypothetical protein Tb11.02.1010 |
| (ZP_01340904) Burkholderia mallei | (XP_762346) Ustilago maydis 521 |
| 200272120 hypothetical protein | hypothetical protein UM06199.1 |
| Bmal2_0300121 | (XP_759106) Ustilago maydis 521 |
| (ZP_01332703) Burkholderia pseudomallei | hypothetical protein UM02959.1 |
| 406e | (XP_3995) Gibberella zeae PH-1 |
| hypothetical protein Bpse4_0300419 | (anamorph: Fusarium graminearum) |
| (ZP_01331490) Burkholderia pseudomallei | hypothetical protein FG09719.1 |
| S13 hypothetical protein BpseS_03000527 | (XP_35376) Gibberella zeae PH-1 |
| (ZP_01322137) Burkholderia pseudomallei | hypothetical protein FG05200.1 |
| Pasteur | (XP_20525) Trypanosoma cruzi strain CL |
| hypothetical protein BpseP_03004099 | Brener |
| (ZP_0131546) Burkholderia pseudomallei | hypothetical protein |
| 1655 | (XP_20057) Trypanosoma cruzi strain CL |
| hypothetical protein Bpse1_03005179 | Brener |
| (YP_52476) Rhodoferax ferrireducens T11 | hypothetical protein |
| acyl-coA-binding protein, ACBP | (XP_16762) Trypanosoma cruzi strain CL |
| (XP_12363) Trypanosoma cruzi strain CL | Brener |
| Brener | hypothetical protein |
| hypothetical protein | (XP_15250) Trypanosoma cruzi strain CL |
| (XP_076) Trypanosoma cruzi strain CL | Brener |
| Brener | hypothetical protein |
| hypothetical protein | (XP_67506) Plasmodium berghei strain |
| (XP_07537) Trypanosoma cruzi strain CL | ANKA |
| Brener | hypothetical protein |
| hypothetical protein | (XP_675761) Plasmodium berghei strain |
| (XP_05136) Trypanosoma cruzi strain CL | ANKA |
| Brener | hypothetical protein |
| hypothetical protein | (XP_66443) Cryptosporidium hominis |
| (XP_02763) Trypanosoma cruzi strain CL | TU502 |
| Brener | proteasome 26S subunit |
| hypothetical protein | (EAS31425) Coccidioides immitis RS |
| (XP_57161) Cryptococcus neoformans var. | hypothetical protein CIMG_06904 |
| neoformans JEC21 (Filobasidiella | (EAS2919) Coccidioides immitis RS |
| neoformans var. neoformans strain JEC21) | predicted protein |
| hypothetical protein | (YP_55032) acyl-coA-binding protein, |
| (XP_570515) Cryptococcus neoformans | ACBP |
| var. neoformans JEC21 (Filobasidiella | acyl-coA-binding protein, ACBP |
| neoformans var. neoformans strain JEC21) | (ABE45934) Polaromonas sp. JS666 |
| long-chain fatty acid transporter | acyl-coA-binding protein, ACBP |
| (XP_5645) Cryptococcus neoformans var. | (ABE31302) Burkholderia xenovorans |
| neoformans JEC21 (Filobasidiella | LB400 |
| neoformans var. neoformans strain JEC21) | Putative acyl-CoA-binding protein |
| long-chain fatty acid transporter | (XP_505020) Yarrowia lipolytica CLIB122 |
| (XP_44404) Candida glabrata CBS 13 | hypothetical protein |
| unnamed protein product | (ABE19190) Sequence 70 from patent U.S. Pat. No. |
| (BAD93154) Homo sapiens (human) | 6,991,901 |
| peroxisomal D3,D2-enoyl-CoA isomerase | (ABE1917) Sequence 67 from patent U.S. Pat. No. |
| isoform 1 variant | 6,991,901 |
| (XP_954032) Theileria annulata strain | (ABE1916) Sequence 66 from patent U.S. Pat. No. |
| Ankara | 6,991,901 |
| hypothetical protein TA0615 | (ABE15645) Sequence 3167 from patent |
| (XP_72944) Plasmodium yoelii yoelii str. | U.S. Pat. No. 6,979,557 |
| 17XNL | XP_970549) Tribolium castaneum (red |
| hypothetical protein PY01656 | flour beetle) |
| (XP_766266) Theileria parva strain | PREDICTED: similar to CG33713-PB, |
| Muguga | isoform B |
| hypothetical protein TP01_0745 | (XP_97424) Tribolium castaneum (red flour |
| (XP_765594) Theileria parva strain | beetle) |
| Muguga | PREDICTED: similar to CG49-PA |
| hypothetical protein TP01_0067 | (XP_97413) Tribolium castaneum (red flour |
| (XP_737124) Plasmodium chabaudi | beetle) PREDICTED: similar to diazepam |
| chabaudi | binding inhibitor |
| hypothetical protein PC000423.03.0 | (XP_970195) Tribolium castaneum (red |
| (XP_7335) Plasmodium chabaudi chabaudi | flour beetle) |
| hypothetical protein PC000150.00.0 | PREDICTED: similar to CG14-PA |
| (NP_90277) Chromobacterium violaceum | (XP_972065) Tribolium castaneum (red |
| ATCC 12472 | flour beetle) |
| probable acyl-CoA-binding protein | PREDICTED: similar to CG14232-PA |
| (NP_901056) Chromobacterium violaceum | (EAL62340) Dictyostelium discoideum AX4 |
| ATCC 12472 | hypothetical protein DDBDRAFT_01797 |
| probable esterase | membrane-associated diazepam binding |
| (YP_513344) Francisella tularensis subsp. | inhibitor; MA-DBI |
| holarctica | (AAB50915) Mus sp. endozepine-like |
| fusion product of 3-hydroxacyl-CoA | peptide; ELP |
| dehydrogenase and acyl-CoA-binding | (AAB36333) Gallus gallus (chicken) acyl- |
| protein | coenzyme A binding protein, ACBP |
| (ZP_0092954) Burkholderia pseudomallei | (AAC06123) Anas platyrhynchos acyl- |
| 1106b | coenzyme A binding protein, ACBP |
| hypothetical protein Bpse110_0200403 | (AAB36332) Testudinidae (tortoises) |
| (ZP_0124423) Flavobacterium johnsoniae | acyl-coenzyme A binding protein, ACBP |
| UW101 | (AAB36331) Canis familiaris (dog) |
| Acyl-coA-binding protein, ACBP | acyl-coenzyme A binding protein, ACBP |
| (EAS60452) Flavobacterium johnsoniae | (AAB30502) Rattus sp. |
| UW101 | DBI39-75 = diazepam binding inhibitor |
| Acyl-coA-binding protein, ACBP | (AAB217) Sus scrota (pig) |
| (CAJ6291) Oryza sativa (indica cultivar- | diazepam-binding inhibitor(32-6); DBI(32-6) |
| group) | (AAM66045) Arabidopsis thaliana (thale |
| H0124B04. | cress) |
| (CAJ623) Oryza sativa (indica cultivar- | putative acyl-CoA binding protein |
| group) | (AAM65019) Arabidopsis thaliana (thale |
| H0901F07.20 | cress) |
| (ZP_01209662) Burkholderia pseudomallei | putative acyl-CoA binding protein |
| 1710a | (YP_369593) Burkholderia sp. 33 |
| hypothetical protein Bpse17_02005312 | Acyl-CoA-binding protein, ACBP |
| (ABD71237) Rhodoferax ferrireducens T11 | (YP_130412) Photobacterium profundum |
| acyl-coA-binding protein, ACBP | SS9 |
| (EAQ7267) Chaetomium globosum CBS | Hypothetical Acyl-CoA-binding protein |
| 14.51 | (AAI0175) Rattus norvegicus (Norway rat) |
| hypothetical protein CHGG_036 | Acbd5 protein |
| (EAQ31) Chaetomium globosum CBS | (ZP_009610) Burkholderia dolosa AUO15 |
| 14.51 | COG421: Acyl-CoA-binding protein |
| hypothetical protein CHGG_10222 | (ZP_00979942) Burkholderia cenocepacia |
| (AAH90641) Mus musculus (house mouse) | PC14 |
| Acbd5 protein | COG421: Acyl-CoA-binding protein |
| (AAH454) Mus musculus (house mouse) | (AAQ59062) Chromobacterium violaceum |
| Acbd5 protein | ATCC 12472 |
| (AAH61029) Mus musculus (house mouse) | probable esterase |
| Acbd6 protein | (AAQ6074) Chromobacterium violaceum |
| (AAH34702) Homo sapiens (human) | ATCC 12472 probable acyl-CoA-binding |
| PECI protein | protein |
| (AAB2237) Manduca sexta (tobacco | (BAE5636) Aspergillus oryzae unnamed |
| hornworm) | protein product |
| diazepam binding inhibitor; DBI | (2COP_A) Homo sapiens (human)Chain A, |
| (AAB21311) Bos taurus (cattle) | Solution Structure Of Rsgi Ruh-040, An |
| (ZP_0093270) Burkholderia mallei JHU | Acbp Domain From Human Cdna |
| COG421: Acyl-CoA-binding protein | (ZP_00945597) Ralstonia solanacearum |
| (ZP_00927796) Burkholderia mallei FMH | UW551 Acyl-CoA-binding protein homolog |
| COG421: Acyl-CoA-binding protein | (EAP71943) Ralstonia solanacearum |
| (ABC13667) Sequence 70 from patent US | UW551 Acyl-CoA-binding protein homolog |
| 696449 | (AAZ2212) Macaca mulatta (rhesus |
| (ABC13664) Sequence 67 from patent US | monkey) |
| 696449 | diazepam-binding protein |
| (ABC13663) Sequence 66 from patent US | (AAZ2211) Nomascus gabriellae (Red- |
| 696449 | cheeked Gibbon) |
| (AAZ22453) Saccharomyces cerevisiae | diazepam-binding protein |
| (baker's yeast)Acb1p | (AAZ2210) Hylobates lar (common gibbon) |
| (ABB0949) Burkholderia sp. 33 | diazepam-binding protein |
| Acyl-CoA-binding protein, ACBP | (AAZ2209) Symphalangus syndactylus |
| (EAN0232) Trypanosoma brucei | (siamang)diazepam-binding protein |
| acyl-CoA binding protein, putative | (AAZ220) Pongo pygmaeus (orangutan) |
| (EAN79322) Trypanosoma | diazepam-binding protein |
| brucei hypothetical protein, conserved | (AAZ2207) Gorilla gorilla (gorilla) |
| (AAZ1273) Trypanosoma brucei | diazepam-binding protein |
| hypothetical protein, conserved | (AAZ2206) Pan paniscus (pygmy |
| (AAX69467) Trypanosoma brucei | chimpanzee) |
| hypothetical protein, conserved | diazepam-binding protein |
| (AAW46941) Cryptococcus neoformans | (AAZ2205) Pan troglodytes (chimpanzee) |
| var. neoformans JEC21 (Filobasidiella | diazepam-binding protein |
| neoformans var. neoformans strain JEC21) | (XP_721591) Candida albicans SC5314 |
| long-chain fatty acid transporter, putative | hypothetical protein CaO19_9202 |
| (AAW44311) Cryptococcus neoformans | (XP_721711) Candida albicans SC5314 |
| var. neoformans JEC21 (Filobasidiella | hypothetical protein CaO19_1634 |
| neoformans var. neoformans strain JEC21) | (XP_66350) Aspergillus nidulans FGSC A4 |
| expressed protein | hypothetical protein AN5904.2 |
| (AAW4320) Cryptococcus neoformans var. | (ZP_00769323) C:hloroflexus aurantiacus |
| neoformans JEC21 (Filobasidiella | J-10-fl |
| neoformans var. neoformans strain JEC21) | Acyl-coA-binding protein, ACBP |
| long-chain fatty acid transporter, putative | (EAO57572) Chloroflexus aurantiacus J- |
| (AAZ221) Ateles geoffroyi (black-handed | 10-fl |
| spider monkey) | Acyl-coA-binding protein, ACBP |
| diazepam-binding protein | (XP_45177) Kluyveromyces lactis NRRL Y- |
| (AAZ2217) Saguinus labiatus (red-chested | 1140 |
| mustached tamarin) | unnamed protein product |
| diazepam-binding protein | (XP_45102) Kluyveromyces lactis NRRL Y- |
| (AAZ2216) Cercopithecus cephus | 1140 |
| (moustached monkey)diazepam-binding | unnamed protein product |
| protein | (XP_53573) Canis familiaris (dog) |
| (AAZ2215) Erythrocebus patas (red | PREDICTED: similar to peroxisomal |
| guenon) | D3,D2-enoyl-CoA isomerase isoform 1 |
| diazepam-binding protein | (XP_50165) Canis familiaris (dog) |
| (AAZ2214) Papio anubis (olive baboon) | PREDICTED: similar to diazepam binding |
| diazepam-binding protein | inhibitor |
| (AAZ2213) Macaca nemestrina (pig-tailed | (XP_49337) Canis familiaris (dog) |
| macaque) | PREDICTED: similar to Acyl-CoA-binding |
| diazepam-binding protein | protein (ACBP) (Diazepam binding |
| (XP_533322) Canis familiaris (dog) | inhibitor) (DBI) (Endozepine) (EP) isoform 4 |
| PREDICTED: similar to diazepam binding | Hypothetical protein F26A1.15 |
| inhibitor isoform 3 | (CAI73962) Theileria annulata |
| (XP_537760) Canis familiaris (dog) | acyl-coa-binding protein, putative |
| PREDICTED: similar to endozepine-like | (CAI73355) Theileria annulata hypothetical |
| peptide | protein, conserved |
| (XP_5565) Canis familiaris (dog) | (EAN9674) Trypanosoma cruzi |
| PREDICTED: similar to acyl-Coenzyme A | hypothetical protein, conserved |
| binding domain containing 4 isoform 2 | (EAN9206) Trypanosoma cruzi |
| (XP_54051) Canis familiaris (dog) | hypothetical protein, conserved |
| PREDICTED: similar to acyl-Coenzyme A | (EAN94911) Trypanosoma cruzi |
| binding domain containing 4 isoform 1 | hypothetical protein, conserved |
| (XP_54705) Canis familiaris (dog) | (EAN94561) Trypanosoma cruzi acyl-CoA |
| PREDICTED: similar to acyl-Coenzyme A | binding protein, putative |
| binding domain containing 3 | (EAN93399) Trypanosoma cruzi |
| (XP_537152) Canis familiaris (dog) | hypothetical protein, conserved |
| similar to acyl-Coenzyme A binding domain | (EAN9133) Trypanosoma cruzi |
| containing 6 | acyl-CoA binding protein, putative |
| (XP_499) Canis familiaris (dog) | (EAN90512) Trypanosoma cruzi |
| PREDICTED: similar to Acyl-CoA-binding | hypothetical protein, conserved |
| protein (ACBP) (Diazepam binding | (EAN253) Trypanosoma cruzi acyl-CoA |
| inhibitor) (DBI) (Endozepine) (EP) | binding protein, putative |
| (XP_535171) Canis familiaris (dog) | (EAN6935) Trypanosoma cruzi |
| PREDICTED: similar to acyl-Coenzyme A | hypothetical protein, conserved |
| binding domain containing 5 isoform 1 | (EAN566) Trypanosoma cruzi hypothetical |
| (XP_5732) Canis familiaris (dog) | protein, conserved |
| PREDICTED: similar to acyl-Coenzyme A | (EAN325) Trypanosoma cruzi hypothetical |
| binding domain containing 5 | protein, conserved |
| isoform 4 | (EAN1317) Trypanosoma cruzi |
| (XP_40) Canis familiaris (dog) | hypothetical protein, conserved |
| PREDICTED: similar to acyl-Coenzyme A | (EAN3393) Theileria parva |
| binding domain containing 5 isoform 3 | hypothetical protein |
| (EAL3352) Drosophila pseudoobscura | (AAH917) Xenopus laevis (African clawed |
| GA21120-PA | frog) |
| (EAL33216) Drosophila pseudoobscura | Unknown (protein for IMAGE: 700255) |
| GA21340-PA | (AAY5103) Drosophila melanogaster (fruit |
| (EAL32791) Drosophila | fly) |
| pseudoobscura GA17261-PA | IP02950p |
| (EAL31626) Drosophila pseudoobscura | (NP_59620) Schizosaccharomyces pombe |
| GA1245-PA | 972h- |
| (EAL3041) Drosophila | hypothetical protein SPBC1539.06 |
| pseudoobscura GA21220-PA | (ZP_0043161) Burkholderia mallei GB |
| (EAL3040) Drosophila pseudoobscura | horse 4 |
| GA13977-PA | COG421: Acyl-CoA-binding protein |
| (EAL3039) Drosophila pseudoobscura | (ZP_00426957) Burkholderia vietnamiensis |
| GA2121-PA | G4 |
| (EAL30591) Drosophila | Acyl-coA-binding protein, ACBP |
| pseudoobscura GA19142-PA | (EAM26450) Burkholderia vietnamiensis |
| (AAZ61305) Ralstonia eutropha JMP134 | G4 Acyl-coA-binding protein, ACBP |
| Acyl-coA-binding protein, ACBP | (BAD96723) Homo sapiens (human) |
| (AAZ3209) Caenorhabditis elegans | (CAH7967) Plasmodium chabaudi |
| peroxisomal D3,D2-enoyl-CoA isomerase | hypothetical protein PC000423.03.0 |
| isoform 1 variant | (CAH74502) Plasmodium chabaudi |
| (BAD96337) Homo sapiens (human) | hypothetical protein PC000150.00.0 |
| peroxisomal D3,D2-enoyl-CoA isomerase | (CAH96600) Plasmodium berghei |
| isoform 1 variant | conserved hypothetical protein |
| (AAY1473) Homo sapiens (human) | (CAI0006) Plasmodium berghei |
| unknown | conserved hypothetical protein |
| (CAG33049) Homo sapiens (human) | (BAC999) Oryza sativa (japonica cultivar- |
| PECI | group) |
| (CAA97025) Saccharomyces cerevisiae | putative Acyl-CoA binding protein (ACBP) |
| (baker's yeast) | (BAD67765) Oryza sativa (japonica |
| ACB1 | cultivar-group) |
| (CAA43673) Mus musculus (house mouse) | putative Acyl-CoA-binding protein |
| diazepam-binding inhibitor | (CAH92214) Pongo pygmaeus (orangutan) |
| (CAE03429) Oryza sativa (japonica | hypothetical protein |
| cultivar-group) | (CAH92157) Pongo pygmaeus (orangutan) |
| OSJNBa0032F06.12 | hypothetical protein |
| (CAD2730) Aspergillus fumigatus possible | (CAH90619) Pongo pygmaeus (orangutan) |
| endozepine | hypothetical protein |
| (CAB66577) Homo sapiens (human) | (BAD67905) Oryza sativa (japonica |
| CAB66577 | cultivar-group) |
| (CAB5133) Schizosaccharomyces pombe | putative Acyl-CoA-binding protein |
| (fission yeast) | (EAL3203) Cryptosporidium hominis similar |
| SPBC1539.06 | to proteasome 26S subunit, non-ATPase, |
| (CAD23129) Gallus gallus (chicken) | 10-related |
| diazepam binding inhibitor | (AAH2394) Xenopus laevis (African clawed |
| (CAC21172) Sus scrofa (pig) | frog) |
| diazepam binding inhibitor | MGC171 protein |
| (CAB56694) Digitalis lanata Acyl-CoA | (EAA57767) Aspergillus nidulans FGSC A4 |
| binding protein (ACBP) | hypothetical protein AN5904.2 |
| (CAB56693) Digitalis lanata | (AAH7350) Xenopus laevis (African clawed |
| Acyl-CoA binding protein (ACBP) | frog) |
| (AAX29010) synthetic construct | MGC277 protein |
| peroxisomal D3D2-enoyl-CoA isomerase | (EAL02923) Candida albicans SC5314 |
| (1ST7_A) Saccharomyces cerevisiae | hypothetical protein CaO19.1634 |
| (baker's yeast) | (EAL02795) Candida albicans SC5314 |
| Chain A, Solution Structure Of Acyl | hypothetical protein CaO19.9202 |
| Coenzyme A Binding Protein | (EAL20326) Cryptococcus neoformans var. |
| (S63593) Testudines gen. sp. | neoformans B-3501Ahypothetical protein |
| (turtle) acyl-coenzyme A-binding protein | CNBF1370 |
| (S63594) Anas platyrhynchos (mallard) | (BAD2667) Plutella xylostella |
| acyl-coenzyme A-binding protein | (diamondback moth) |
| (CAG32737) Gallus gallus (chicken) | Diazepam binding inhibitor-like protein |
| hypothetical protein | (1NVL_A) Bos taurus (cattle) |
| (CAG32279) Gallus gallus (chicken) | Chain A, Rdc-Refined Nmr Structure Of |
| hypothetical protein | Bovine Acyl-Coenzyme A Binding Protein, |
| (CAH909) Plasmodium chabaudi | Acbp, In Complex With Palmitoyl- |
| conserved hypothetical protein | Coenzyme A |
| Chain A, Rdc-Refined Nmr Structure Of | (1NTI_A) Bos taurus (cattle) |
| Bovine Acyl-Coenzyme A Binding Protein, | unknown protein |
| Acbp | (AAR37334) Helicoverpa armigera (cotton |
| (CAG33237) Homo sapiens (human) | bollworm) |
| DBI | diazepam-binding inhibitor |
| (CAG05340) Tetraodon nigroviridis | (CAE90961) Homo sapiens (human) |
| unnamed protein product | unnamed protein product |
| (CAF97634) Tetraodon nigroviridis | (CAE59601) Caenorhabditis |
| unnamed protein product | briggsae Hypothetical protein CBG03009 |
| (CAG1190) Tetraodon nigroviridis | (CAE7079) Caenorhabditis |
| unnamed protein product | briggsae Hypothetical protein CBG1755 |
| (CAG0912) Tetraodon nigroviridis | (CAE71345) Caenorhabditis briggsae |
| unnamed protein product | Hypothetical protein CBG1247 |
| (CAG05477) Tetraodon nigroviridis | (CAE71116) Caenorhabditis briggsae |
| unnamed protein product | Hypothetical protein CBG17969 |
| (CAG1225) Tetraodon nigroviridis | (CAE73560) Caenorhabditis briggsae |
| unnamed protein product | Hypothetical protein CBG21030 |
| (AAT00460) Cyprinus carpio endozepine | (CAE73559) Caenorhabditis briggsae |
| (AAS93766) Drosophila melanogaster (fruit | Hypothetical protein CBG2102 |
| fly) | (CAE744) Caenorhabditis briggsae |
| GM17572p | Hypothetical protein CBG22239 |
| (EAK7103) Ustilago maydis 521 | (CAE6127) Caenorhabditis briggsae |
| hypothetical protein UM06199.1 | Hypothetical protein CBG13772 |
| (EAK4131) Ustilago maydis 521 | (CAE57625) Caenorhabditis |
| hypothetical protein UM02959.1 | briggsae Hypothetical protein CBG0060 |
| (AAS76751) Arabidopsis thaliana (thale | (CAE67722) Caenorhabditis briggsae |
| cress) | Hypothetical protein CBG13297 |
| At4g24230 | (CAE69296) Caenorhabditis briggsae |
| (CAF6353) Homo sapiens (human) | Hypothetical protein CBG15351 |
| unnamed protein product | (CAE57136) Caenorhabditis briggsae |
| (CAF6344) Homo sapiens (human) | Hypothetical protein CBG25061 |
| unnamed protein product | (AAR09996) Drosophila yakuba similar to |
| (EAI05740) environmental sequence (cf. | Drosophila melanogaster CG49 |
| Burkholderia SAR-1) | (BAC5726) Oryza sativa (japonica cultivar- |
| unknown | group) |
| (EAH3112) environmental sequence | putative Acyl-CoA binding protein (ACBP) |
| unknown | (AAQ96259) Rattus norvegicus (Norway |
| (AAS21130) Arabidopsis thaliana (thale | rat) LRRGT00046 |
| cress) | (AAP94639) Rattus norvegicus (Norway |
| At4g24230 | rat) |
| (EAA7776) Gibberella zeae PH-1 | DMT1-associated protein |
| (anamorph: Fusarium graminearum) | (AAP97271) Homo sapiens (human) |
| hypothetical protein FG09719.1 | benzodiazepine receptor ligand |
| (EAA74077) Gibberella zeae PH-1 | (AAP6251) Rattus norvegicus (Norway rat) |
| (anamorph: Fusarium graminearum) | Ac1-130 |
| hypothetical protein FG05200.1 | (AAP3775) Arabidopsis thaliana (thale |
| (AAR50) Oryza sativa (japonica cultivar- | cress) |
| group) putative transcription factor | At5g27630 |
| (AAF64540) Arabidopsis thaliana (thale | (AAP36349) synthetic construct Homo |
| cress) | sapiens peroxisomal D3,D2-enoyl-CoA |
| (AAP21266) Arabidopsis thaliana (thale | isomerase |
| cress)At3g05420 | (AAK93155) Drosophila melanogaster (fruit |
| (AAN60219) Homo sapiens (human) | fly) LD25952p |
| peripherial benzodiazepine receptor | (AAF79123) Callithrix jacchus (white-tufted- |
| associated protein | ear marmoset) |
| (AAM2215) Mus musculus (house mouse) | endozepine-like protein |
| peripherial benzodiazepine receptor | (AAF79120) Macaca fascicularis (crab- |
| associated protein PAP7 | eating macaque)endozepine-like protein |
| (EAA33144) Neurospora crassa predicted | (AAF7911) Bos taurus (cattle) |
| protein | endozepine-like protein |
| (EAA27950) Neurospora | (AAF79124) Mus musculus (house mouse) |
| crassa hypothetical protein | endozepine-like protein |
| (AAO20903) Takifugu rubripes (Fugu | (AAF66247) Homo sapiens (human) |
| rubripes) | hepatocellular carcinoma-associated |
| carnitine octanoyltransferase | antigen |
| (AAB71197) Mus musculus (house mouse) | (AAD32606) Rattus norvegicus (Norway |
| peripherial benzodiazepine receptor | rat) |
| associated protein; PBR associated | endozepine-like peptide |
| protein; PAP7 | (AAC19317) Homo sapiens (human) |
| (AAF6974) Homo sapiens (human) | DBI-related protein |
| hepatocellular carcinoma-associated | (AAD34174) Mus musculus (house mouse) |
| antigen 64 | peroxisomal D3,D2-enoyl-CoA isomerase |
| (BAC02705) Homo sapiens (human) | (AAD34173) Homo sapiens (human) |
| KIAA1996 protein | peroxisomal D3,D2-enoyl-CoA isomerase |
| (AAM13155) Arabidopsis thaliana (thale | (AAD32607) Rattus norvegicus (Norway |
| cress) unknown protein | rat) endozepine-like peptide |
| (AAM1332) Arabidopsis thaliana (thale | (I96735) Sequence 5 from patent US |
| cress) | 573403 |
| unknown protein | (I96734) Sequence 4 from patent US |
| (AAL4930) Drosophila melanogaster (fruit | 573403 |
| fly) | (I96733) Sequence 3 from patent US |
| RE33457p | 573403 |
| (AAL4575) Drosophila melanogaster (fruit | (BAA34531) Sus scrofa (pig) |
| fly) | endozepine |
| RE05521p | |
| (AAL4175) Drosophila melanogaster (fruit | |
| fly) | |
| RH39533p | |
| (CAD19062) Homo sapiens (human) | |
| unnamed protein product | |
| (BAB20592) Homo sapiens (human) | |
| golgi resident protein GCP60 | |
| (AAL2451) Drosophila melanogaster (fruit | |
| fly) GM05135p | |
| (AAL24363) Arabidopsis thaliana (thale | |
| cress) | |
| Unknown protein | |
| (AAC1940) Cyprinus carpio ACBP/ECHM | |
| (AAK93272) Drosophila melanogaster (fruit | |
| fly) LD3507p | |
| (AAB60606) Rana ridibunda (marsh frog) | |
| diazepam-binding inhibitor | |
| (1911410A) Bos taurus (cattle) | |
| membrane-associated diazepam-binding | |
| inhibitor | |
| (1411307A) Rattus norvegicus (Norway rat) | |
| diazepam binding inhibitor | |
| (AAA52171) Homo sapiens (human) | |
| diazepam binding inhibitor | |
| (AAA41079) Rattus norvegicus (Norway | |
| rat) diazepam binding inhibitor | |
| (AAA4107) Rattus norvegicus (Norway rat) | |
| diazepam binding inhibitor | |
| (AAA357) Homo sapiens (human) | |
| endozepine precursor | |
| (AAA30496) Bos taurus (cattle) | |
| endozepine-related protein precursor | |
| (AAA30495) Bos taurus (cattle) | |
| endozepine precursor | |
| (AAA29309) Manduca sexta (tobacco | |
| hornworm) | |
| diazepam binding inhibitor-like peptide | |
| (AAA21650) Drosophila melanogaster (fruit | |
| fly) | |
| diazepam binding inhibitor | |
| (AAA21649) Drosophila melanogaster (fruit | |
| fly) | |
| diazepam binding inhibitor | |

TABLE 2
The following table provides further promoters that may be used in accordance with
the present disclosure

| SEQ ID | | | Consensus Sequence | |
|---|---|---|---|---|
| NO: | Promoter | Element | (motifs) | Nucleotides |
| 34 | Flax 16 kDa | RY | CATGCA(C/T) | 1818-1824 |
| | oleosin | ABRE | (G/C/T)ACGT(G/T)GC | 1859-1867 |
| | | | ACACGTGGC | 1858-1867 |
| | | G-Box | CACGTG | 1745-1751 |
| | | | | 1879-1885 |
| | | E-Box | CANNTG | 172-177 |
| | | | | 548-554 |
| | | | | 942-948 |
| | | | | 1405-1410 |
| | | | | 1467-1473 |
| | | | | 1816-1822 |
| 35 | Flax 18 kDa | RY | CATGCA | 1658-1663 |
| | oleosin | ABRE | ATGGATTTG (van Rooijen | 1203-1212 |
| | | | et al., 2004; Patent | |
| | | | 6,718,554) | |
| | | G-Box | CACGTG | 1690-1696 |
| | | E-Box | CANNTG | 175-181 |
| | | | | 245-250 |
| | | | | 366-372 |
| | | | | 416-422 |
| | | | | 559-565 |
| | | | | 777-783 |
| | | | | 798-804 |
| | | | | 1074-1080 |
| | | | | 1690-1696 |
| 36 | Arabidopsis 18 kDa | RY | CATGCA | 916-922 |
| | oleosin | ABRE | (G/C/T)ACGT(G/T)GC | 946-954 |
| | | | AAACGTGTC | 882-891 |
| | | | ACACGTGGC | 945-954 |
| | | G-Box | CACGTG | 946-952 |
| | | | | 980-986 |
| | | E-Box | CANNTG | 82-88 |
| | | | | 500-506 |
| | | | | 915-920 |
| | | | | 957-963 |
| 37 | Bean Phaseolin | RY | CATGCA(C/T) | 1074-1081 |
| | | | | 1234-1241 |
| | | | | 1401-1408 |
| | | ABRE | G-Box and coupling element | 1223-1228 |
| | | | (CE; CACACGTC motif) | and |
| | | | Comprise the ABA response | 1357-1364 |
| | | | (Kawagoe et al., 1994) | |
| | | G-Box | CACGTG | 1223-1229 |
| | | E-Box | CANNTG | 521-527 |
| | | | | 612-618 |
| | | | | 663-669 |
| | | | | 699-705 |
| | | | | 1308-1314 |
| | | | | 1371-1377 |
| 38 | Brassica napus | RY | CATGCA | 925-931 |
| | napin promoter | | | |
| | | | | 1047-1053 |
| | | | CATGCA(C/T) | 1024-1031 |
| | | ABRE | GCCACTTGTC (Ezcurra et | 955-964 |
| | | | al., 2000; Plant Journal 24: | |
| | | | 57-66) | |
| | | G-Box | CACGTG | 1036-1042 |
| | | E-Box | CANNTG | 181-187 |
| | | | | 216-222 |
| | | | | 257-263 |
| | | | | 447-453 |
| | | | | 500-506 |
| | | | | 593-599 |
| | | | | 956-962 |
| | | | | 1014-1020 |
| | | | | 1037-1042 |
| 39 | Brassica napus | RY | CATGCA | 366-372 |
| | cruciferin promoter | | | 1647-1653 |
| | GenBank M93103 | | | 1664-1670 |
| | | ABRE | Expression responsive to | |
| | | | ABA (Wilen et al., 1991; | |
| | | | Plant Physiol 95: 399-405) | |
| | | G-Box | CACGTG | 442-448 |
| | | E-Box | CANNTG | 526-532 |
| | | | | 1528-1534 |
| | | | | 1615-1621 |
| | | | | 1764-1770 |
| | | | | 1889-1895 |
| 40 | Brassica cruciferin | RY | CATGCA | 373-379 |
| | SBS derived | | | 1654-1660 |
| | | | | 1671-1677 |
| | | ABRE | Expression responsive to | |
| | | | ABA (Wilen et al., 1991; | |
| | | | Plant Physiol 95: 399-405) | |
| | | | ACACNNG (Kim et al., | 384-391 |
| | | | 1997, Plant J. 11: 1237-1251) | |
| | | G-Box | CACGTG | 449-454 |
| | | E-Box | CANNTG | 533-539 |
| | | | | 1536-1542 |
| | | | | 1622-1628 |
| | | | | 1771-1777 |
| | | | | 1897-1903 |
| 41 | Flax legumin-like | RY | CATGCA | 1260-1266 |
| | (linin) promoter | | | |
| | | | | 1888-1894 |
| | | | CATGCA(C/T) | 662-669 |
| | | | | 1880-1887 |
| | | ABRE | Uncharacterized with respect | Putative sites |
| | | | to ABA responsiveness or | (core ACTG |
| | | | defined ABRE or A BRC | present) may |
| | | | | be |
| | | | | 584-588 |
| | | | | 674-678 |
| | | | | 957-961 |
| | | | | 1036-1040 |
| | | | | 1184-1188 |
| | | | | 1224-1228 |
| | | | ACACNNG (Kim et al., | 1017-1024 |
| | | | 1997, Plant J. 11: 237-1251) | |
| | | | | 1762-1769 |
| | | | | 1835-1841 |
| | | G-Box | CACGTG | 1224-1230 |
| | | E-Box | CANNTG | 182-188 |
| | | | | 948-954 |
| | | | | 1084-1900 |
| | | | | 1198-1204 |
| | | | | 1213-1219 |
| | | | | 1223-1229 |
| | | | | 1688-1694 |
| | | | | 1836-1842 |
| 107 | bean arcelin | RY | CATGCA(C/T) | 1387-1394 |
| | promoter (arc5-1) | | | |
| | | | | 1474-1481 |
| | | | | 1527-1534 |
| | | | | 1578-1565 |
| | | ABRE | (G/C/T)ACGT(G/T)GC | ABA |
| | | | | responsive |
| | | | | Kermode et |
| | | | | al., 2007, |
| | | | | Plant Mol |
| | | | | Biol. |
| | | | | 63: 763-776 |
| | | | ACACNNG (Kim et al., | 983-990; |
| | | | 1997, Plant J. 11: 1237-1251) | 1420-1427 |
| | | G-Box | CACGTG | 1486-1492 |
| | | E-box | CANNTG | 754-760 |
| | | | | 1226-1332 |
| | | | | 1370-1376 |
| | | | | 1421-1427 |
| | | | | 1486-1492 |
| | | | | 1778-1784 |

TABLE 3
Oil Body Protein Motif (Amino Acid Sequence Identifier) {Nucleic Acid Sequence Identifier}

Oleosin
(A84654) Arabidopsis thaliana probable oleosin
(AAA87295) Arabidopsis thaliana oleosin {Gene L40954}
(AAC42242) Arabidopsis thaliana oleosin {Gene AC005395}
(AAF01542) Arabidopsis thaliana putative oleosin {Gene AC009325}
(AAF69712) Arabidopsis thaliana F27J15.22 {Gene AC016041}
(AAK96731) Arabidopsis thaliana oleosin-like protein {Gene AY054540}
(AAL14385) Arabidopsis thaliana AT5g40420/MPO12_130 oleosin isoform {Gene AY057590}
(AAL24418) Arabidopsis thaliana putative oleosin {Gene AY059936}
(AAL47366) Arabidopsis thaliana oleosin-like protein {Gene AY064657}
(AAM10217) Arabidopsis thaliana putative oleosin {Gene AY081655}
(AAM47319) Arabidopsis thaliana AT5g40420/MPO12_130 oleosin isoform {Gene AY113011}
(AAM63098) Arabidopsis thaliana oleosin isoform {Gene AY085886}
(AAO22633) Arabidopsis thaliana putative oleosin {Gene BT002813}
(AAO22794) Arabidopsis thaliana putative oleosin protein {Gene BT002985}
(AAO42120) Arabidopsis thaliana putative oleosin {Gene BT004094}
(AAO50491) Arabidopsis thaliana putative oleosin {Gene BT004958}
(AAO63989) Arabidopsis thaliana putative oleosin {Gene BT005569}
(AAQ56108) Arabidopsis lyrata subsp. Lyrata Oleosin. {Gene AY292860}
(BAA97384) Arabidopsis thaliana oleosin-like {Gene AB023044}
(BAB02690) Arabidopsis thaliana oleosin-like protein {Gene AB018114}
(BAB11599) Arabidopsis thaliana oleosin, isoform 21K {Gene AB006702}
(BAC42839) Arabidopsis thaliana putative oleosin protein {Gene AK118217}
(CAA44225) Arabidopsis thaliana oleosin {Gene X62353}
(CAA63011) Arabidopsis thaliana oleosin, type 4 {Gene X91918}
(CAA63022) Arabidopsis thaliana oleosin, type 2 {Gene X91956}
(CAA90877) Arabidopsis thaliana oleosin {Gene Z54164}
(CAA90878) Arabidopsis thaliana oleosin {Gene Z54165}
(CAB36756) Arabidopsis thaliana oleosin, 18.5K {Gene AL035523}
(CAB79423) Arabidopsis thaliana oleosin, 18.5K {Gene AL161562}
(CAB87945) Arabidopsis thaliana oleosin-like protein {Gene AL163912}
(P29525) Arabidopsis thaliana oleosin 18.5 kDa {Gene X62353, CAA44225,
AL035523, CAB36756, CAB36756, CAB79423, Z17738, S22538}
(Q39165) Arabidopsis thaliana Oleosin 21.2 kDa (Oleosin type 2). {Gene L40954,
AAA87295, X91956, CAA63022, Z17657, AB006702, BAB11599, AY057590, AAL14385, S71253
(Q42431) Arabidopsis thaliana Oleosin 20.3 kDa (Oleosin type 4) {Gene
Z54164, CAA90877, X91918, CAA63011, AB018114, BAB02690, AY054540,
AAK96731, AY064657, AAL47366, AY085886, AAM63098, Z27260, Z29859, S71286
(Q43284) Arabidopsis thaliana Oleosin 14.9 kDa. {Gene Z54165, CAA90878,
AB023044, BAA97384, Z27008, CAA81561}
(S22538) Arabidopsis thaliana oleosin, 18.5K
(S71253) Arabidopsis thaliana oleosin, 21K
(S71286) Arabidopsis thaliana oleosin, 20K
(T49895) Arabidopsis thaliana oleosin-like protein
(AAB22218) Brassica napus oleosin napII
(AAD24547) Brassica oleracea oleosin
(CAA43941) Brassica napus oleosin BN-III {Gene X63779}
(CAA45313) Brassica napus oleosin BN-V {Gene X63779}
(P29109) Brassica napus Oleosin Bn-V (BnV) {Gene X63779, CAA45313, S25089)
(P29110) Brassica napus Oleosin Bn-III (BnIII) {Gene X61937, CAA43941, S22475)
(P29111) Brassica napus Major oleosin NAP-II {Gene X58000, CAA41064, S70915)
(S22475) Brassica napus oleosin BN-III
(S50195) Brassica napus Oleosin
(T08134) Brassica napus Oleosin-like
(AAB01098) Daucus carota oleosin
(T14307) carrot oleosin
(A35040) Zea mays oleosin 18
(AAA67699)Zea mays oleosin KD18 {Gene J05212}
(AAA68065) Zea mays 16 kDa oleosin {Gene U13701}
(AAA68066) Zea mays 17 kDa oleosin {Gene U13702}
(P13436) Zea mays OLEOSIN ZM-I (OLEOSIN 16 KD) (LIPID BODY-ASSOCIATED MAJOR
PROTEIN) {Gene U13701, AAA68065, M17225, AAA33481, A29788}
(P21641) Zea mays Oleosin Zm-II (Oleosin 18 kDa) (Lipid body-associated
protein L2) {Gene J05212, AAA67699, A35040}
(S52029) Zea mays oleosin 16
(S52030) Zea mays oleosin 17
Caleosin
(XP_467656) putative caleosin [Oryza sativa (japonica cultivar-group)].
(BAD16161) putative caleosin [Oryza sativa (japonica cultivar-group)]. {Gene AP005319}
(NP_973892) caleosin-related family protein [Arabidopsis thaliana]. {Gene NM_202163}
(NP_564996) caleosin-related family protein [Arabidopsis thaliana]. {Gene NM_105736}
(NP_564995) caleosin-related family protein [Arabidopsis thaliana}{Gene NM_105735}
(NP_200335) caleosin-related family protein/embryo-specific protein,
putative [Arabidopsis thaliana]. {Gene NM_124906}
(NP_173739) caleosin-related [Arabidopsis thaliana].{Gene NM_102174}
(NP_173738) caleosin-related family protein [Arabidopsis thaliana]{Gene NM_102173}
(AAQ74240) caleosin 2 [Hordeum vulgare]. {Gene AY370892}
(AAQ74239) caleosin 2 [Hordeum vulgare]. {Gene AY370891}
(AAQ74238) caleosin 1 [Hordeum vulgare]. {Gene AY370890}
(AAQ74237) caleosin 1 [Hordeum vulgare]. {Gene AY370889}
(AAF13743) caleosin [Sesamum indicum]. {Gene AF109921}
Steroleosin
(XP_465935) putative steroleosin [Oryza sativa (japonica cultivar-group)]. {Gene XM_465935}
(XP_465933) putative steroleosin [Oryza sativa (japonica cultivar-group)]. {Gene XM_465933}
(AAT77030) putative steroleosin-B [Oryza sativa (japonica cultivar-group)]. {Gene AC096856}
(BAD23084) putative steroleosin [Oryza sativa (japonica cultivar-group)] {Gene AP004861}
(BAD23082) putative steroleosin [Oryza sativa (japonica cultivar-group)] {Gene AP004861}
(AAM46847) steroleosin-B [Sesamum indicum]. {Gene AF498264}
(AAL13315) steroleosin [Sesamum indicum]. {Gene AF421889}
(AAL09328) steroleosin [Sesamum indicum]. {Gene AF302806}
oleosin Arabidopsis thaliana (SEQ ID NO: 79)
oleosin Brassica napus (SEQ ID NO: 80)
caleosin Arabidopsis thaliana (SEQ ID NO: 81).
caleosin Arabidopsis thaliana (SEQ ID NO: 82).
steroleosin Sesamum indicum (SEQ ID NO: 83),

TABLE 4
T-DNA vectors

| | Unique | | | | |
|---|---|---|---|---|---|
| | cloning | LacZ | | | |
| Vector | sites | selection? | Resistance in | | |
| pBIN19 | 9 | no | kan | kan | Bevan M. 1984, Binary Agrobacterium |
| | | | | | vectors for plant transformation. Nucleic |
| | | | | | Acids Res. 12: 8711-8721. |
| pCAMBIA | variable | yes (not | chlor, | hyg, kan | http://www.cambia.org.au |
| series | | all) | kan | | |
| pCGN | 5 | yes | gent | kan | McBride K. E. and Summerfelt K. R. 1990. |
| series | | | | | Improved binary vectors for Agrobacterium- |
| | | | | | mediated plant transformation. Plant Mol. |
| | | | | | Biol. 14: 269-276. |
| pJJ/pSLJ | 5-11 | yes | tet | bar, kan, | Jones J. D., Shlumukov L., Carland F., English |
| series | | | | hyg, spec | J., Scofield S. R., Bishop G. J., Harrison K. |
| | | | | | 1992. Effective vectors for transformation, |
| | | | | | expression of heterologous genes, and |
| | | | | | assaying transposon excision in transgenic |
| | | | | | plants. Transgenic Res. 1: 285-297. |
| pPZP series | 9 | yes | chlor, | kan, gent | Hajdukiewicz P., Svab Z., Maliga P. 1994. |
| | | | spec | | The small, versatile pPZP family of |
| | | | | | Agrobacterium binary vectors for plant |
| | | | | | transformation. Plant Mol. Biol. 25: 989-994. |
| pGreen | 18 | yes | kan | bar, kan, | Hellens R P, Edwards E A, Leyland N R, Bean |
| series | | | | hyg, sul | S, Mullineaux P M. 2000a. pGreen: A |
| | | | | | versatile and flexible binary Ti vector for |
| | | | | | Agrobacterium-mediated plant |
| | | | | | transformation. Plant Mol. Biol. 42: 819-832. |

TABLE 5
Seed oil content of T₂ lines determined
by gravimetric method (Grav) and by GC.

| | | Oil content (Grey), | Oil content (GC), |
|---|---|---|---|
| Construct | T2 line | % weight | % weight |
| WT | N/A | 30.57 ± 0.60 | 31.29 ± 0.48 |
| D9-ACBP-1-KDEL | 5-10 | 33.16 ± 0.57 | 32.71 ± 0.45 |
| ACBP-1 | 6-06 | 32.54 ± 0.80 | 34.84 ± 0.41 |
| | 6-08 | 32.71 ± 0.23 | 32.62 ± 0.44 |
| D9-ACBP-1 | 7-09 | 33.70 ± 0.17 | 39.51 ± 1.21 |
| | 7-11 | 38.29 ± 0.86 | 40.69 ± 0.96 |

Mean % weight ± SE (n = 4).

TABLE 6
Changes in composition of FA classes in seed oil of
A. thaliana T₂ mature seeds comparing to WT.

| Construct | SFA | MUFA | PUFA |
|---|---|---|---|
| WT | 12.98 ± 0.06 | 38.67 ± 0.30 | 48.34 ± 0.24 |
| Null Segr-ACBP-1 | +0.21/−0.00 | +0.48/−0.19 | +0.08/−0.68 |
| PhaP-Oleosin-ACBP-1 | −1.18 | +1.90 | −0.96 |
| PhaP-ACBP-1-Oleosin * | +0.57/−1.63 | +2.35/−4.97 | +4.37/−0.82 |
| PhaP-B82-Oleosin-ACBP-1 * | +2.58/−0.69 | +1.06/−6.50 | +4.44/−0.49 |
| PhaP-OleosinH3P-ACBP-l * | −0.26/−1.45 | +1.75/−3.11 | +3.37/−0.29 |
| PhaP-ACBP-1-KDEL * | −0.17/−1.17 | +1.53/−2.77 | +2.95/−0.60 |
| PhaP-D9-ACBP-1-KDEL | +0.66/−1.24 | +2.97/−0.53 | +0.45/−1.73 |
| PhaP-ACBP-1 * | −0.81/−2.48 | +2.48/−2.89 | +4.81/−1.15 |
| PhaP-D9-ACBP-1 | −0.31/−1.85 | +3.67/−0.17 | +1.00/−1.82 |
| 35S-ACBP-1 | +0.90/−2.10 | +5.74/+0.03 | +0.81/−3.98 |
| 35S-ACBP-1-Oleosin | +1.80/−1.63 | +2.36/−2.40 | +0.63/−3.83 |
| 35S-ACBP-1-KDEL | +1.49/−1.77 | +4.79/−1.47 | −0.01/−4.95 |

Mean % weight ± SD for WT; max/min difference between mean % weight abs. values of T₂ line within construct and WT. Constructs marked with the asterisk (*) contain T₂ lines with increased PUFA % weight, comparing to WT (α = 0.05)

TABLE 7
Composition of FA classes in A. thaliana T2 seeds.

| Construct | SFA | MUFA | PUFA |
|---|---|---|---|
| WT | 13.33 ± 0.46 | 38.29 ± 0.69 | 48.34 ± 0.23 |
| Null Segr- | 12.81 ± 0.09 | 38.09 ± 0.32 | 49.05 ± 0.34 |
| ACBP-1 | | | |
| PhaP-Oleosin- | 11.79 ± 0.06 | 40.58 ± 0.18 | 47.38 ± 0.39 |
| ACBP-1 | | | |
| PhaP-ACBP- | 12.98 ± 0.49 | 35.28 ± 1.11^{▾} | 51.74 ± 0.75^{▴} |
| Oleosin-1 | | | |
| PhaP-B82- | 14.71 ± 1.1^{▴} | 32.65 ± 1.16^{▾} | 52.58 ± 0.49^{▴} |
| Oleosin-ACBP-1 | | | |
| PhaP- | 12.25 ± 0.41^{▾} | 36.59 ± 1.23^{▾} | 50.80 ± 0.82^{▴} |
| OleosinH3P- | | | |
| ACBP-1 | | | |
| PhaP-ACBP- | 12.44 ± 0.46 | 37.64 ± 1.26 | 49.38 ± 0.93^{▴} |
| 1-KDEL | | | |
| PhaP-D9- | 12.90 ± 0.51 | 38.72 ± 0.60 | 48.37 ± 0.28 |
| ACBP-1-KDEL | | | |
| PhaP-ACBP-1 | 11.15 ± 0.73^{▾} | 36.50 ± 0.58^{▾} | 52.35 ± 0.79^{▴} |
| PhaP-D9- | 12.08 ± 0.23^{▾} | 39.08 ± 0.60 | 48.84 ± 0.42 |
| ACBP-1 | | | |
| 35S-ACBP-1 | 12.64 ± 0.18^{▾} | 40.00 ± 0.30^{▴} | 46.66 ± 0.25^{▾} |
| 35S-ACBP-1- | 13.27 ± 0.18 | 38.00 ± 0.30 | 48.20 ± 0.25 |
| Oleosin | | | |
| 35S-ACBP-1- | 12.98 ± 0.19 | 39.79 ± 0.31^{▴} | 47.17 ± 0.26^{▾} |
| KDEL | | | |

Four lines with the highest PUFA % in seed oil within each construct are included in the analysis (For PhaP-Oleosin-ACBP construct only one T2 line was analyzed), mean % weight + SD of the means (n = 4).
^{▴/▾}values significantly greater/smaller than WT at α = 0.05.

TABLE 8
FA composition of the seed oil from A. thaliana T₂ seeds.
Four T₂ lines with the highest PUFA % in seed oil within each construct were included in the analysis.

| Construct | 16:0 | 18:0 | 18:1 | 18:2 | 18:3 | 20:1 |
|---|---|---|---|---|---|---|
| WT | 7.08 ± 0.41 | 3.81 ± 0.13 | 15.15 ± 0.01 | 27.08 ± 0.15 | 19.52 ± 0.11 | 19.99 ± 0.76 |
| Null Segr-ACBP-1 | 7.41 ± 0.05 | 3.33 ± 0.08 | 15.07 ± 0.71 | 27.23 ± 0.40 | 19.72 ± 0.63 | 19.99 ± 0.35 |
| PhaP-Oleosin-ACBP-1 | 7.44 ± 0.06 | 2.59 ± 0.02^{▾} | 18.24 ± 0.20^{▴} | 30.44 ± 0.16^{▴} | 15.40 ± 0.47^{▾} | 18.66 ± 0.23^{▾} |
| PhaP-ACBP-1-Oleosin | 7.78 ± 0.45^{▴} | 2.82 ± 0.19^{▾} | 16.04 ± 1.19 | 30.98 ± 0.33^{▴} | 18.17 ± 1.00^{▾} | 17.35 ± 0.14^{▾} |
| PhaP-B82-Oleosin-ACBP-1 | 9.70 ± 0.82^{▴} | 3.18 ± 0.32^{▾} | 14.56 ± 0.91 | 33.77 ± 1.51^{▴} | 17.38 ± 1.03^{▾} | 14.71 ± 1.45^{▾} |
| PhaP-OleosinH3P-ACBP-1 | 7.46 ± 0.16 | 2.84 ± 0.14^{▾} | 16.04 ± 1.19 | 30.98 ± 0.33^{▴} | 18.17 ± 1.00^{▾} | 17.35 ± 0.14^{▾} |
| PhaP-ACBP-1-KDEL | 7.55 ± 0.33^{Δ} | 2.99 ± 0.20^{▾} | 15.45 ± 0.78 | 32.67 ± 0.68^{▴} | 15.53 ± 0.29^{▾} | 18.99 ± 0.66^{▾} |
| PhaP-D9-ACBP-1-KDEL | 7.98 ± 0.36^{▴} | 2.91 ± 0.11^{▾} | 16.64 ± 0.65^{▴} | 31.20 ± 0.48^{▴} | 15.48 ± 0.76^{▾} | 18.36 ± 0.70^{▾} |
| PhaP-ACBP-1 | 6.36 ± 0.34^{▾} | 2.89 ± 0.27^{▾} | 15.81 ± 0.38 | 29.22 ± 0.74^{▴} | 21.32 ± 1.14^{▴} | 17.45 ± 0.49^{▾} |
| PhaP-D9-ACBP-1 | 7.26 ± 0.12 | 2.83 ± 0.09^{▾} | 16.19 ± 1.02 | 30.23 ± 0.55^{▴} | 16.83 ± 0.66^{▾} | 19.28 ± 0.32⋄ |
| 35S-ACBP-1 | 7.58 ± 0.44 | 3.12 ± 0.34^{▾} | 18.40 ± 2.00^{▴} | 27.34 ± 1.46 | 19.26 ± 0.60 | 17.88 ± 2.52^{▾} |
| 35S-ACBP-1-Oleosin | 7.81 ± 0.41 | 3.33 ± 0.67 | 15.84 ± 1.52 | 27.14 ± 2.10 | 19.53 ± 0.56 | 19.49 ± 1.61 |
| 35S-ACBP-1-KDEL | 7.85 ± 0.45 | 3.14 ± 0.37^{▾} | 16.56 ± 1.80 | 26.57 ± 1.76 | 20.05 ± 0.29^{▴} | 19.04 ± 1.53 |

% weight ± SD of the means [n(T₂) = 4, biological replicates of each construct;
n = 4, technical replicates of each T₂line].
(^{▴}/^{▾}) values significantly greater/smaller than WT at α = 0.05;
(^{Δ}/⋄) values significantly greater/smaller than WT at α = 0.

TABLE 9
Composition of FA classes in A. thaliana T₃ seeds.

| Construct | SFA | MUFA | PUFA |
|---|---|---|---|
| WT | 13.74 ± 0.49 | 39.09 ± 0.38 | 47.11 ± 0.21 |
| PhaP-ACBP-1- | 13.10 ± 0.33^{▾} | 37.43 ± 1.29 | 49.41 ± 1.06^{▴} |
| Oleosin | | | |
| PhaP-OleoH3P- | 13.45 ± 0.21 | 37.45 ± 1.04 | 49.05 ± 0.4^{Δ} |
| ACBP-1 | | | |
| PhaP-ACBP-1- | 13.74 ± 0.23 | 39.38 ± 1.16 | 46.82 ± 0.95 |
| KDEL | | | |
| PhaP-D9-ACBP- | 12.96 ± 0.41^{▾} | 41.37 ± 2.07^{Δ} | 45.6 ± 1.66 |
| 1-KDEL | | | |
| PhaP-ACBP-1 | 11.43 ± 0.22^{▾} | 38.33 ± 0.88 | 50.17 ± 0.78^{▴} |
| PhaP-D9-ACBP- | 12.43 ± 0.13^{▾} | 41.63 ± 0.66^{▴} | 45.87 ± 0.52 |
| 1 | | | |

Mean % weight ± SD of the means [n(T₂lines) = 4, biological replicates of each construct; n(T₃lines) = 10, biological replicates of each T₂line].
^{▴/▾}values significantly greater/smaller than WT at α = 0.05;
^{Δ}values significantly greater than WT at α = 0.1.

TABLE 10
FA composition of seed oil of T3 seeds from the top four A. thaliana T2 lines
(lines with the highest PUFA % in seed oil) within each construct.

| Construct | 16:0 | 18:0 | 18:1 | 18:2 | 18:3 | 20:1 |
|---|---|---|---|---|---|---|
| WT | 7.33 ± 0.09 | 3.93 ± 0.31 | 15.09 ± 0.09 | 27.53 ± 0.14 | 17.92 ± 0.09 | 19.06 ± 0.34 |
| PhaP-ACBP-1-Oleosin | 8.14 ± 0.33^{▴} | 2.77 ± 0.11^{▾} | 15.57 ± 1.38 | 31.52 ± 1.66^{▴} | 16.23 ± 1.54 | 16.14 ± 0.97^{▾} |
| PhaP-OleosinH3P-ACBP-1 | 8.34 ± 0.20^{▴} | 3.09 ± 0.10^{▾} | 16.20 ± 0.97 | 31.39 ± 0.29^{▴} | 16.10 ± 0.61⋄ | 15.48 ± 0.34^{▾} |
| PhaP-ACBP-1-KDEL | 8.67 ± 0.17^{▴} | 3.09 ± 0.12^{▾} | 16.54 ± 1.01 | 32.52 ± 1.34^{▴} | 12.80 ± 1.06^{▾} | 16.92 ± 0.95^{▾} |
| PhaP-D9-ACBP-1-KDEL | 8.09 ± 0.29^{▴} | 3.00 ± 0.08^{▾} | 18.09 ± 2.20^{▴} | 30.43 ± 0.28^{▴} | 13.68 ± 1.70^{▾} | 16.91 ± 0.39^{▾} |
| PhaP-ACBP-1 | 6.85 ± 0.08^{▾} | 2.79 ± 0.18^{▾} | 16.29 ± 0.61 | 29.33 ± 0.77^{Δ} | 19.06 ± 0.72 | 15.84 ± 0.63^{▾} |
| PhaP-D9-ACBP-1 | 7.55 ± 0.06 | 2.94 ± 0.11^{▾} | 17.82 ± 0.88^{▴} | 30.00 ± 0.69^{▴} | 14.27 ± 1.05^{▾} | 18.30 ± 0.38 |

Mean % weight + SD of the means [n(T2lines) = 4, biological replicates of each construct; n(T3lines) = 10, biological replicates of each T2line].
(^{▴}/^{▾}) values significantly greater/smaller than WT at α = 0.05;
(^{Δ}/^{⋄}) values significantly greater/smaller than WT at α = 0.1.

1. A method for increasing the level of polyunsaturated fatty acids and/or oil in plants comprising:
(a) providing a chimeric nucleic acid construct comprising, in the 5′ to 3′ direction of transcription as operably linked components:
i. a nucleic acid sequence capable of controlling expression in plant cells in a seed-preferred manner; and
ii. a nucleic acid sequence encoding an acyl CoA binding protein,
(b) introducing the chimeric nucleic acid construct into a plant cell; and
(c) growing the plant cell into a mature plant capable of setting seed wherein the acyl-CoA binding protein is expressed in the seed.

2. The method of claim 1 wherein the nucleic acid sequence capable of controlling expression in a plant seed cell is a seed preferred promoter.

3. The method of claim 2 wherein the seed preferred promoter comprises an ABRE promoter element sequence.

4. The method according to claim 3 wherein the ABRE sequence comprises a nucleic acid sequence selected from the group of nucleic acid sequences consisting of: (1) ACGT, (2) (G/C/T)ACGT(G/T)GC, (3) (C/T)ACGTGGC, (4) TGACGTGGG, (5) AAACGTGTC, (6) ACACGTGGC, (7) ACACCTGAC) and (8) ACACNNG.

5. The method according to claim 2 wherein the seed preferred promoter further comprises an RY repeat.

6. The method according to claim 2 wherein the seed preferred promoter further comprises a promoter element selected from the group of promoter elements consisting of G-Box and E-Box.

7. The method of claim 1 wherein the chimeric nucleic acid construct further comprising a sequence encoding a stabilizing polypeptide.

8. The method of claim 7 wherein the stabilizing polypeptide comprises an antibody that binds to an oilbody protein.

9. The method according to claim 8 wherein the antibody is a single chain antibody.

10. The method according to claim 1 wherein the acyl CoA binding protein accumulates in the cytosol.

11. The method according to claim 1 wherein the acyl CoA binding protein has the amino acid sequence of any one of SEQ ID NOS:1-33.

12. The method according to claim 1 wherein the chimeric nucleic acid construct further comprises a nucleic acid sequence encoding an oil body protein.

13. The method according to claim 1 wherein the seed preferred promoter is selected from phaseolin, oleosin, linin, napin, crusiferin or arcelin.

14. The method according to claim 13 wherein the seed-preferred promoter is phaseolin.

15. The method according to claim 1 further comprising (a) obtaining seed from the plant wherein the seed comprises increased levels of polyunsaturated fatty acids (PUFAs) and/or oil relative to a control.

16. The method according to claim 15 wherein the PUFA levels are increased by no less than 1% relative to the control wherein the control is a wild type plant.

17. The method according to claim 15 wherein the PUFA levels are increased by no less than 4% relative to the control wherein the control is a wild type plant.

18. The method according to claim 15 wherein the oil levels are increased by no less than 5% relative to the control wherein the control is a wild type plant.

19. The method according to claim 15 wherein the oil levels are increased by no less than 9% relative to the control wherein the control is a wild type plant.

20. The method according to claim 1 wherein the plant is selected from the group consisting of peanut (Arachis hypogaea); mustard (Brassica spp. and Sinapis alba); rapeseed (Brassica spp.); chickpea (Cicer arietinum); soybean (Glycine max); cotton (Gossypium hirsutum); sunflower (Helianthus annuus); lentil (Lens culinaris); linseed/flax (Linum usitatissimum); white clover (Trifolium repens); olive (Olea eurpaea); oil palm (Elaeis guineensis); safflower (Carthamus tinctorius); false flax (Camelina sp.); borage or starflower (Borago officinalis); evening primrose (Oenothera spp); and narbon bean (Vicia narbonesis).

21. The method according to claim 20 wherein the plant is Arabidopsis or Brassica.

22. A chimeric nucleic acid construct comprising in the 5′ to 3′ direction of transcription:
(a) a first nucleic acid sequence capable of controlling expression in a plant cell in a seed-preferred manner operatively linked to;
(b) a second nucleic acid sequence encoding an acyl-CoA binding protein polypeptide.

23. The chimeric nucleic acid construct of claim 12 wherein the nucleic acid sequence capable of controlling expression in a plant seed cell is a seed preferred promoter.

24. The chimeric nucleic acid construct of claim 23 wherein the seed preferred promoter comprises an ABRE promoter element sequence.

25. The chimeric nucleic acid construct according to claim 24 wherein the ABRE sequence comprises a nucleic acid sequence selected from the group of nucleic acid sequences consisting of: (1) ACGT, (2) (G/C/T)ACGT(G/T)GC, (3) (C/T)ACGTGGC, (4) TGACGTGGG, (5) AAACGTGTC, (6) ACACGTGGC, (7) ACACCTGAC) and (8) ACACNNG.

26. The chimeric nucleic acid construct according to claim 23 wherein the seed preferred promoter further comprises an RY repeat.

27. The chimeric nucleic acid construct according to claim 23 wherein the seed preferred promoter further comprises a promoter element selected from the group of promoter elements consisting of G-Box and E-Box.

28. The chimeric nucleic acid construct of claim 23 wherein the chimeric nucleic acid construct further comprising a sequence encoding a stabilizing polypeptide.

29. The chimeric nucleic acid construct of claim 28 wherein the stabilizing polypeptide comprises an antibody that binds to an oilbody protein.

30. The chimeric nucleic acid construct according to claim 29 wherein the antibody is a single chain antibody.

31. The chimeric nucleic acid construct according to claim 23 wherein the acyl CoA binding protein accumulates in the cytosol.

32. The chimeric nucleic acid construct according to claim 23 wherein the acyl CoA binding protein has the amino acid sequence of any one of SEQ ID NOS:1-33.

33. The method according to claim 23 wherein the chimeric nucleic acid construct further comprises a nucleic acid sequence encoding an oil body protein.

34. The method according to claim 23 wherein the seed preferred promoter is selected from phaseolin, oleosin, linin, napin, crusiferin or arcelin.

35. The method according to claim 34 wherein the seed-preferred promoter is phaseolin.

36. A plant cell of a plant capable of setting seed, the cell comprising a chimeric nucleic acid sequence according to claim 22.

37. The plant cell of claim 36 wherein the chimeric nucleic acid is part of the cell's nuclear genome.

38. The plant cell of claim 36 wherein the plant is an Arabidopsis plant, a Carthamus plant, or a Brassica plant.

39. A plant seed comprising a plant cell according to claim 36.

40. The plant seed according to claim 39 wherein the seed comprises increased levels of polyunsaturated fatty acids (PUFAs) and/or oil relative to a control.

41. The plant seed according to claim 40 wherein the PUFA levels are increased by no less than 1% relative to the control wherein the control is a wild type plant.

42. The plant seed according to claim 40 wherein the PUFA levels are increased by no less than 4% relative to the control wherein the control is a wild type plant.

43. The plant seed according to claim 40 wherein the oil levels are increased by no less than 5% relative to the control wherein the control is a wild type plant.

44. The plant seed according to claim 40 wherein the oil levels are increased by no less than 9% relative to the control wherein the control is a wild type plant.

45. The plant seed according to claim 39 wherein the plant is selected from the group consisting of peanut (Arachis hypogaea); mustard (Brassica spp. and Sinapis alba); rapeseed (Brassica spp.); chickpea (Cicer arietinum); soybean (Glycine max); cotton (Gossypium hirsutum); sunflower (Helianthus annuus); lentil (Lens culinaris); linseed/flax (Linum usitatissimum); white clover (Trifolium repens); olive (Olea eurpaea); oil palm (Elaeis guineensis); safflower (Carthamus tinctorius); false flax (Camelina sp.); borage or starflower (Borago officinalis); evening primrose (Oenothera spp); and narbon bean (Vicia narbonesis).

46. The plant seed according to claim 45 wherein the plant is Arabidopsis or Brassica.
